(19) United States
(12) Patent Application Publication
(10) Pub. No.: US 2026/0130988 A1
(43) Pub. Date: May 14, 2026

(54) SARS-CoV-2 universal recombinant antigen polypeptides, polynucleotides and uses thereof

(71) Applicant: Korea National Institute of Health, Cheongju-si (KR)

(72) Inventors: Mi Ran YUN, Cheongju-si (KR); Do Keun KIM, Cheongju-si (KR); You Jin KIM, Cheongju-si (KR); Yun Ho HWANG, Cheongju-si (KR); Jin Ah YEO, Cheongju-si (KR); Ji Hyang JEON, Cheongju-si (KR); Si Hyun KIM, Cheongju-si (KR); Tae Young LEE, Cheongju-si (KR); Seo Yeon KIM, Cheongju-si (KR); Ki Soon KIM, Seoul (KR); Man Seong PARK, Seoul (KR); Jong Hyeon SEOK, Seoul (KR)

(21) Appl. No.: 19/344,363

(22) Filed: Sep. 29, 2025

(30) Foreign Application Priority Data
Nov. 8, 2024 (KR) 10-2024-0157677

(51) Int. Cl. A61K 39/215; A61P 37/04

(52) U.S. Cl. CPC A61K 39/215; A61P 37/04

(57) ABSTRACT

Proposed herein are a polypeptide including a SARS-CoV-2 universal antigen amino acid sequences, and a polynucleotide encoding the polypeptide. Also proposed herein are a vaccine composition for preventing SARS-CoV-2 infection, and a pharmaceutical composition for preventing or treating SARS-CoV-2 infection, wherein the vaccine and pharmaceutical compositions contain the polypeptide or polynucleotide as an active ingredient. The present invention provides a universal antigen polypeptide capable of responding not only to wild-type SARS-CoV-2 and currently circulating SARS-CoV-2 variants, but also to hypothetical SARS-CoV-2 variants, as well as a polynucleotide encoding the same. In addition, the present invention provides a SARS-CoV-2 universal antigen polypeptide with enhanced structural stability through amino acid substitutions at specific positions of the universal antigen polypeptide, and a polynucleotide encoding the same. Furthermore, the present invention provides a vaccine composition and a pharmaceutical composition comprising the SARS-CoV-2 universal antigen polypeptide or the polynucleotide as an active ingredient.

### BACKGROUND OF THE DISCLOSURE

### 1. Field of the Disclosure

The present disclosure relates to a Severe Acute Respiratory Syndrome-Coronavirus 2 (SARS-CoV-2) universal antigen polypeptide and polynucleotide, and uses thereof. More specifically, the present disclosure relates to a polypeptide containing SARS-CoV-2 universal antigen amino acid sequences, and a polynucleotide encoding the polypeptide. Furthermore, the present disclosure relates to a vaccine composition for preventing SARS-CoV-2 infection, and a pharmaceutical composition for preventing or treating SARS-CoV-2 infection, wherein the compositions contains the aforementioned polypeptide or polynucleotide as an active ingredient.

### 2. Description of the Related Art

As well known in the art, “Coronavirus disease 2019 (COVID-19)” refers to a severe acute respiratory syndrome caused by a pathogen SARS-CoV-2, and SARS-CoV-2 is a zoonotic ribonucleic acid (RNA) virus. RNA viruses are characterized by mutating more readily than DNA viruses, and known to be able to variate into new viral strains exhibiting resistance to vaccines or therapeutic agents.

SARS-CoV-2 has a spherical structure in which its RNA genome is enclosed by an envelope protruding with spike proteins. It is known that the spike proteins of SARS-CoV-2 binds to ACE2 receptors on a host cell membrane, thereby facilitating entry of viral nucleic acids into the cell. Mutations in the spike proteins, in particular in a receptor-binding domain, significantly affect viral infectivity and transmissibility, and may lead to resistance to vaccines and therapeutic agents. To date, major variants that have been reported include the Alpha, Beta, Gamma, Delta, and Omicron variants, and subvariants of Omicron are continuing to emerge.

Currently, globally authorized and administered SARS-CoV-2 vaccines are based on spike proteins of Wuhan strains, which are known as wild type that initially caused SARS-CoV-2 infection. However, these vaccines have not shown effective and sufficient response against various SARS-CoV-2 variants. Accordingly, there is an urgent need to develop universal vaccines and therapeutic agents that exhibit high immunogenicity and efficacy more stably not only against wild-type SARS-CoV-2 and existing variants, but also against potential future variants that may emerge.

### REFERENCE OF RELATED ART

Patent: KR 10-2023-0082071 A

### SUMMARY OF THE INVENTION

Accordingly, the present disclosure has been made keeping in mind the above problems occurring in the related art, and an objective of the present disclosure is to provide a SARS-CoV-2 universal antigen polypeptide and polynucleotide that exhibit high immunogenicity and efficacy more stably not only against the wild-type SARS-CoV-2 and existing variants, but also against potential future variants that may arise.

Another objective of the present disclosure is to provide a polypeptide for preventing or treating SARS-CoV-2 infection, wherein the polypeptide includes a SARS-CoV-2 universal antigen amino acid sequence.

Furthermore, other objective of the present disclosure is to provide a polynucleotide for preventing or treating SARS-CoV-2 infection, wherein the polynucleotide encodes a polypeptide containing a SARS-CoV-2 universal antigen amino acid sequence.

A further objective of the present disclosure is to provide a vaccine for preventing SARS-CoV-2 infection and a pharmaceutical composition for preventing or treating the infection, wherein the vaccine and pharmaceutical composition contains the above polypeptide or polynucleotide as an active ingredient.

The objectives of the present disclosure are not limited to the above descriptions, and the disclosure is intended to encompass all applications that may achieve appropriate effects by utilizing the present disclosure.

The present disclosure relates to a polypeptide containing a SARS-CoV-2 universal antigen amino acid sequence, and a polynucleotide encoding the polypeptide. Furthermore, the present disclosure relates to a vaccine composition for preventing SARS-CoV-2 infection, and a pharmaceutical composition for preventing or treating such an infection, wherein the vaccine composition and pharmaceutical composition contains the polypeptide or polynucleotide as an active ingredient.

In order to develop vaccines and therapeutic agents that can be universally applied to various SARS-CoV-2 variants, the present inventors analyzed approximately 2,000 sequences including all existing genotypes of the SARS-CoV-2 variants, specifically S1 subunits of SARS-CoV-2 spike proteins which serve as a major antigen of the virus. Through this analysis, the present inventors derived a conserved consensus sequence, and thereby completing the present disclosure relating to a SARS-CoV-2 universal antigen amino acid sequence. Furthermore, to enhance a structural stability of the SARS-CoV-2 universal antigen polypeptide, the present inventors completed the disclosure relating an optimized SARS-CoV-2 universal antigen amino acid sequence with improved structural stability of the universal antigen polypeptide, by substituting specific amino acids at designated positions within the universal antigen amino acid sequence. In particular, it was confirmed that amino acid substitutions at positions 173 (Q173) and 256 (S256) function as universal stabilization sites, enhancing the expression and structural stability of the spike protein. Furthermore, when the double substitution Q173S and S256Q was introduced, the most significant increase in expression and stabilization effect was observed, and this effect was consistently validated even in Omicron subvariants, including the JN.1 variant. These results demonstrate that the stabilization design strategy of the present disclosure is not limited to specific variants but represents a universal principle applicable across SARS-CoV-2 variants.

In addition, the present inventors produced a SARS-CoV-2 universal antigen polypeptide and a polynucleotide encoding the same, by utilizing amino acid sequences of the SARS-CoV-2 universal antigen polypeptide, and a polynucleotide encoding the same, and also produced vaccines and therapeutic agents containing these polypeptide or polynucleotide as an active ingredient. As a result, the inventors completed a universal vaccines and therapeutic agents capable of responding not only to the wild-type SARS-CoV-2 and existing variants thereof, but also to potential future variants of SARS-CoV-2.

Specifically, the present disclosure provides a polypeptide for preventing or treating SARS-CoV-2 infection, wherein the polypeptide includes one or more amino acid substitutions relative to a reference amino acid sequence of SEQ ID NO: 1, as mentioned below.

The amino acid substitutions include one or more selected from a group consisting of: substitution of an amino acid corresponding to position 27 with E; substitution of an amino acid corresponding to position 50 with M; substitution of an amino acid corresponding to position 52 with T; substitution of an amino acid corresponding to position 73 with S; substitution of an amino acid corresponding to position 78 with Y; substitution of an amino acid corresponding to position 132 with Y; substitution of an amino acid corresponding to position 161 with D; substitution of an amino acid corresponding to position 173 with S or K; substitution of an amino acid corresponding to position 213 with T; substitution of an amino acid corresponding to position 256 with Q or N; substitution of an amino acid corresponding to position 260 with V; and substitution of an amino acid corresponding to position 324 with D.

The present disclosure also provides a polypeptide for preventing or treating SARS-CoV-2 infection, wherein the amino acid substitutions include: substitution of an amino acid corresponding to position 173 with S or K; and substitution of an amino acid corresponding to position 256 with Q or N.

Furthermore, the present disclosure provides a polypeptide for preventing or treating SARS-CoV-2 infection, wherein the amino acid substitutions include: substitution of an amino acid corresponding to position 52 with T; substitution of an amino acid corresponding to position 161 with D; substitution of an amino acid corresponding to position 173 with S; substitution of an amino acid corresponding to position 256 with Q; and substitution of an amino acid corresponding to position 324 with D.

Furthermore, the present disclosure provides a polypeptide for preventing or treating SARS-CoV-2 infection, wherein the amino acid substitutions include: substitution of the amino acid corresponding to position 27 with E; substitution at position 50 with M; substitution at position 73 with S; substitution at position 78 with Y; substitution at position 132 with Y; substitution at position 173 with K; substitution at position 213 with T; substitution at position 256 with N; and substitution at position 260 with V.

The present disclosure also provides a polypeptide for preventing or treating SARS-CoV-2 infection, wherein amino acid substitutions at positions 173 and 256 provide universal stabilization sites that enhance the expression and structural stability of the spike protein.

The present disclosure further provides, according to claim 7, a polypeptide for preventing or treating SARS-CoV-2 infection, wherein the polypeptide comprises double substitutions Q173S and S256Q, which function as universal stabilization sites to improve the expression and structural stability of the spike protein.

The present disclosure also provides a polypeptide for preventing or treating SARS-CoV-2 infection, wherein the polypeptide is based on the spike protein of the JN.1 variant and includes Q173 and S256 substitutions.

Furthermore, the present disclosure provides a polypeptide for preventing or treating SARS-CoV-2 infection, wherein the polypeptide includes amino acid sequence of SEQ ID NO: 2.

Furthermore, the present disclosure provides a polypeptide for preventing or treating SARS-CoV-2 infection, wherein the polypeptide includes amino acid sequence of SEQ ID NO: 3.

Furthermore, the present disclosure provides a polypeptide for preventing or treating SARS-CoV-2 infection, wherein the polypeptide includes amino acid sequence of SEQ ID NO: 4.

Furthermore, the present disclosure provides a polypeptide for preventing or treating SARS-CoV-2 infection, wherein the polypeptide includes amino acid sequence of SEQ ID NO: 5.

Furthermore, the present disclosure provides a polypeptide for preventing or treating SARS-CoV-2 infection, wherein the SARS-CoV-2 includes a variant of SARS-CoV-2.

Furthermore, the present disclosure provides a polypeptide for preventing or treating SARS-CoV-2 infection, wherein the variant of SARS-CoV-2 is one or more selected from a group consisting of the Alpha, Beta, Gamma, Delta, and Omicron variants.

Furthermore, the present disclosure provides a vaccine composition for preventing or treating SARS-CoV-2 infection, wherein the vaccine composition contains the polypeptide as an active ingredient.

Furthermore, the present disclosure provides a pharmaceutical composition for preventing or treating SARS-CoV-2 infection, wherein the pharmaceutical composition contains the polypeptide as an active ingredient.

Furthermore, the present disclosure provides a polynucleotide for preventing or treating SARS-CoV-2 infection, wherein the polynucleotide encodes the polypeptide.

Furthermore, the present disclosure provides a vaccine composition for preventing SARS-CoV-2 infection, wherein the vaccine composition contains the polynucleotide as an active ingredient.

Furthermore, the present disclosure provides a pharmaceutical composition for preventing or treating SARS-CoV-2 infection, wherein the pharmaceutical composition contains the polynucleotide as an active ingredient.

Hereinafter, the present disclosure will be described in more detail.

In a particular embodiment, the present disclosure provides a polypeptide for preventing or treating SARS-CoV-2 infection, wherein the polypeptide includes one or more amino acid substitutions relative to a reference amino acid sequence of SEQ ID NO: 1, and the substitutions are selected from a group consisting of the following:
substitution of an amino acid corresponding to position 27 with E;
substitution of an amino acid corresponding to position 50 with M;
substitution of an amino acid corresponding to position 52 with T;
substitution of an amino acid corresponding to position 73 with S;
substitution of an amino acid corresponding to position 78 with Y;
substitution of an amino acid corresponding to position 132 with Y;
substitution of an amino acid corresponding to position 161 with D;
substitution of an amino acid corresponding to position 173 with S or K;
substitution of an amino acid corresponding to position 213 with T;
substitution of an amino acid corresponding to position 256 with Q or N;
substitution of an amino acid corresponding to position 260 with V; and
substitution of an amino acid corresponding to position 324 with D.

In the present disclosure, the amino acid sequence of SEQ ID NO: 1 is as shown in Table 1 below.

TABLE 1
SARS-COV-2_Control (Wuhan strain) S1 (N→C)
(SEQ ID NO: 1)

MFVFLVLLPLVSSQCVNLTTRTQLPPAYTNSFTRGVYYPDKVFRSSVL
HSTQDLFLPFFSNVTWFHAIHVSGTNGTKRFDNPVLPFNDGVYFASTE
KSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREF
VFKNIDGYFKIYSKHTPINLVRDLPQGFSALEPLVDLPIGINITRFQT
LLALHRSYLTPGDSSSGWTAGAAAYYVGYLQPRTFLLKYNENGTITDA
VDCALDPLSETKCTLKSFTVEKGIYQTSNFRVQPTESIVRFPNITNLC
PFGEVFNATRFASVYAWNRKRISNCVADYSVLYNSASFSTFKCYGVSP
TKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPK
KSTNLVKNKCVNFNFNGLTGTGVLTESNKKFLPFQQFGRDIADTTDAV
RDPQTLEILDITPCSFGGVSVITPGTNTSNQVAVLYQDVNCTEVPVAI
HADQLTPTWRVYSTGSNVFQTRAGCLIGAEHVNNSYECDIPIGAGICA
SYQTQTNSPRRAR

In a particular embodiment of the present disclosure, the amino acid substitution positions were derived by the following methodology. Specifically, the three-dimensional structure of the SARS-CoV-2 spike protein S1 domain was modeled using AlphaFold2, which employs a deep learning technique based on in-silico analysis. Based on the predicted three-dimensional structure and sequence, in-silico analysis was performed using the pSUFER and FuncLib servers to calculate protein sequence variation data and atom-based Rosetta structural energy changes. After that, results of this analysis were used to derive a design repertoire of 1,000 proteins incorporating mutations that enhance a stability of a protein folding structure. Among these, top five amino acid variants were selected as primary candidates. Physicochemical properties such as solubility, cavity formation, and RNA secondary structure, which associate with protein expression levels and mRNA stability of the primary candidates, were further predicted using in-silico tools using Aggrescan 3D (A3D), CASTp-3.0, RNAfold servers, and the like. Final mutation sites were determined by integrating the results of predictions.

In the present disclosure, the amino acid substitution positions are determined relative to a reference amino acid sequence of SEQ ID NO: 1, wherein each position corresponds to the numbering from N-terminus of the amino acid sequence.

In the present disclosure, the term “corresponding to” refers to an amino acid residue located at a position or region that is similar, identical, or homologous to the enumerated position in an amino acid sequence. In a particular embodiment of the present disclosure, when determining substitution positions of amino acids, sequence alignment is performed between an arbitrary amino acid sequence and the reference sequence. Based on the positions numbered from N-terminus of the reference sequence, amino acid residues that are considered similar, identical, or homologous to respective positions or regions, are regarded as “corresponding to” amino acids at the positions, and such amino acids may be substituted accordingly.

The method for performing the sequence alignment is not particularly limited, and may be carried out using sequence alignment programs or pairwise sequence comparison algorithms that are commonly used in the relevant technical field. Non-limiting examples of such sequence alignment programs or algorithms include Needleman-Wunsch algorithm (Needleman and Wunsch, 1970, J. Mol. Biol. 48:443-453), Needle program from the EMBOSS package (EMBOSS: The European Molecular Biology Open Software Suite; Rice et al., 2000, Trends Genet. 16:276-277), and MAFFT program (Katoh et al., 2002, Nucleic Acids Res. 30: 3059-3066).

The polypeptide for preventing or treating SARS-CoV-2 infection according to the present disclosure, may include any amino acid sequence without limitation, wherein one or more amino acid substitutions selected from the aforementioned group are introduced into the amino acid sequence, and the amino acid sequence is aligned with the reference sequence of SEQ ID NO: 1. For achieving superior prophylactic or therapeutic efficacy against SARS-CoV-2 infection, the polypeptide may, in particular, have a sequence homology of 70% or greater but less than 100%, more specifically 80% or greater but less than 100%, and even more specifically 90% or greater but less than 100% with the amino acid sequence of SEQ ID NO: 1.

In the present disclosure, the amino acid substitutions may be 1, 2, 3, 4, 5, 6, 7, 8, 9, 10, 11, or 12 substitutions selected from the aforementioned group. A polypeptide with such amino acid substitutions offers an advantage of further improved structural stability.

In the present disclosure, the amino acid substitutions that most significantly enhance structural stability are, in particular, a substitution of the amino acid corresponding to position 173 with S or K, and a substitution of the amino acid corresponding to position 256 with Q or N.

In yet another embodiment, the present disclosure provides a polypeptide for preventing or treating SARS-CoV-2 infection, wherein the polypeptide includes the substitutions selected from a group consisting of the following:
substitution of an amino acid corresponding to position 52 with T;
substitution of an amino acid corresponding to position 161 with D;
substitution of an amino acid corresponding to position 173 with S;
substitution of an amino acid corresponding to position 256 with Q; and
substitution of an amino acid corresponding to position 324 with D.

In yet another embodiment, the present disclosure provides a polypeptide for preventing or treating SARS-CoV-2 infection, wherein the polypeptide with the above five substitutions includes an amino acid sequence of SEQ ID NO: 2.

In the present disclosure, the amino acid sequence of SEQ ID NO: 2 is as shown in Table 2.

TABLE 2
SARS-COV-2 universal antigen polypeptide
(Consensus_dsg; Css_dsg) (SEQ ID NO: 2)

MFVFLVLLPLVSSQCVNLTTRTQLPPAYTNSFTRGVYYPDKVFRSSVL
HSTTDLFLPFFSNVTWFHAISGTNGTKRFDNPVLPFNDGVYFASTEKS
NIIRGWIFGTTLDSKTQSLLIVNNATNVVIKVCEFQFCNDPFLGVYHK
NNKSWMESEFRVYDSANNCTFEYVSSPFLMDLEGKQGNFKNLREFVFK
NIDGYFKIYSKHTPINLVRDLPQGFSALEPLVDLPIGINITRFQTLLA
LHRSYLTPGDSSQGWTAGAAAYYVGYLQPRTFLLKYNENGTITDAVDC
ALDPLSETKCTLKSFTVEKGIYQTSNFRVQPTDSIVRFPNITNLCPFG
EVFNATRFASVYAWNRKRISNCVADYSVLYNSASFSTFKCYGVSPTKL
NDLCFTNVYADSFVIRGDEVRQIAPGQTGKIADYNYKLPDDFTGCVIA
WNSNNLDSKVGGNYNYRYRLFRKSNLKPFERDISTEIYQAGSKPCNGV
EGFNCYFPLQSYGFQPTNGVGYQPYRVVVLSFELLHAPATVCGPKKST
NLVKNKCVNFNFNGLTGTGVLTESNKKFLPFQQFGRDIADTTDAVRDP
QTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQ
TQTNSPA

In yet another embodiment, the present disclosure provides a polypeptide for preventing or treating SARS-CoV-2 infection, wherein the polypeptide includes substitutions selected from a group consisting of the following:
substitution of an amino acid corresponding to position 27 with E;
substitution of an amino acid corresponding to position 50 with M;
substitution of an amino acid corresponding to position 73 with S;
substitution of an amino acid corresponding to position 78 with Y;
substitution of an amino acid corresponding to position 132 with Y;
substitution of an amino acid corresponding to position 173 with K;
substitution of an amino acid corresponding to position 213 with T;
substitution of an amino acid corresponding to position 256 with N; and
substitution of an amino acid corresponding to position 260 with V.

In yet another embodiment, the present disclosure provides a polypeptide for preventing or treating SARS-CoV-2 infection, wherein the polypeptide with the above nine substitutions includes an amino acid sequence of SEQ ID NO: 3.

In the present disclosure, the amino acid sequence of SEQ ID NO: 3 is as shown in Table 3.

TABLE 3
SARS-COV-2 universal antigen polypeptide
(Chimeric_dsg; Chi_dsg) (SEQ ID NO: 3)

MFVFLVLLPLVSSQCVNLITRTQEYTNSFTRGVYYPDKVFRSSVLHMT
QDLFLPFFSNVTWFHAIHVSGSNGTKYFDNPVLPFNDGVYFASTEKSN
IIRGWIFGTTLDSKTQSLLIVNNATNVVIKVCYFQFCNDPFLDVYYHK
NNKSWMESEFRVYSSANNCTFEYVSKPFLMDLEGKQGNFKNLREFVFK
NIDGYFKIYSKHTPINLTRDLPQGFSALEPLVDLPIGINITRFQTLLA
LHRSYLTPGDSSNGWTVGAAAYYVGYLQPRTFLLKYNENGTITDAVDC
ALDPLSETKCTLKSFTVEKGIYQTSNFRVQPTESIVRFPNITNLCPFH
EVFNATTFASVYAWNRKRISNCVADYSVLYNFAPFFAFKCYGVSPTKL
NDLCFTNVYADSFVIRGNEVSQIAPGQTGNIADYNYKLPDDFTGCVIA
WNSNKLDSKVSGNYNYLYRLFRKSKLKPFERDISTEIYQAGNKPCNGV
AGFNCYFPLQSYGFRPTYGVGHQPYRVVVLSFELLHAPATVCGPKKST
NLVKNKCVNFNFNGLTGTGVLTESNKKFLPFQQFGRDIADTTDAVRDP
QTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQ
TQTNSHA

In yet another embodiment, the present disclosure provides a polypeptide for preventing or treating SARS-CoV-2 infection, wherein the polypeptide includes an amino acid sequence of SEQ ID NOS: 4 or 5.

In the present disclosure, the amino acid sequence of SEQ ID NO: 4 is as shown in Table 4.

TABLE 4
SARS-COV-2 universal antigen polypeptide
(Consensus; Css) (SEQ ID NO: 4)

MFVFLVLLPLVSSQCVNLTTRTQLPPAYTNSFTRGVYYPDKVFRSSVL
HSTQDLFLPFFSNVTWFHAISGTNGTKRFDNPVLPFNDGVYFASTEKS
NIIRGWIFGTTLDSKTQSLLIVNNATNVVIKVCEFQFCNDPFLGVYHK
NNKSWMESEFRVYSSANNCTFEYVSQPFLMDLEGKQGNFKNLREFVFK
NIDGYFKIYSKHTPINLVRDLPQGFSALEPLVDLPIGINITRFQTLLA
LHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFG
EVFNATRFASVYAWNRKRISNCVADYSVLYNSASFSTFKCYGVSPTKL
NDLCFTNVYADSFVIRGDEVRQIAPGQTGKIADYNYKLPDDFTGCVIA
WNSNNLDSKVGGNYNYRYRLFRKSNLKPFERDISTEIYQAGSKPCNGV
EGFNCYFPLQSYGFQPTNGVGYQPYRVVVLSFELLHAPATVCGPKKST
NLVKNKCVNFNFNGLTGTGVLTESNKKFLPFQQFGRDIADTTDAVRDP
QTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQ
TQTNSPA

In the present disclosure, the amino acid sequence of SEQ ID NO: 5 is as shown in Table 5.

TABLE 5
SARS-CoV-2 universal antigen polypeptide
(Chimeric; Chi) (SEQ ID NO: 5)

MFVFLVLLPLVSSQCVNLITRTQSYTNSFTRGVYYPDKVFRSSVLHST
QDLFLPFFSNVTWFHAIHVSGTNGTKRFDNPVLPFNDGVYFASTEKSN
IIRGWIFGTTLDSKTQSLLIVNNATNVVIKVCEFQFCNDPFLDVYYHK
NNKSWMESEFRVYSSANNCTFEYVSQPFLMDLEGKQGNFKNLREFVFK
NIDGYFKIYSKHTPINLGRDLPQGFSALEPLVDLPIGINITRFQTLLA
LHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFH
EVFNATTFASVYAWNRKRISNCVADYSVLYNFAPFFAFKCYGVSPTKL
NDLCFTNVYADSFVIRGNEVSQIAPGQTGNIADYNYKLPDDFTGCVIA
WNSNKLDSKVSGNYNYLYRLFRKSKLKPFERDISTEIYQAGNKPCNGV
AGFNCYFPLQSYGFRPTYGVGHQPYRVVVLSFELLHAPATVCGPKKST
NLVKNKCVNFNFNGLTGTGVLTESNKKFLPFQQFGRDIADTTDAVRDP
QTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQ
TQTNSHA

In the present disclosure, SEQ ID NOS: 4 and 5 were used to design universal antigen sequences generically applicable to various SARS-CoV-2 variants. Specifically, molecular phylogenetic analysis based on Bayesian inference was performed using approximately 2,000 target sequences including all existing genotypes of the SARS-CoV-2 variants, specifically S1 subunits of SARS-CoV-2 spike proteins which serve as a major antigen of the virus. Through this analysis, conserved consensus sequences were derived.

In another embodiment of the present disclosure, a polypeptide consisting of the amino acid sequences of SEQ ID NOS: 4 or 5 is provided, wherein one or more amino acids are substituted to further enhance structural stability of the polypeptide.

In the present disclosure, when sequence alignment is performed with the amino acid sequences of SEQ ID NOS: 4 or 5 using an amino acid sequence of SEQ ID NO: 1 as a reference sequence, the amino acid substitutions include one or more selected from a group consisting of: substitution of an amino acid corresponding to position 27 with E; substitution of an amino acid corresponding to position 50 with M; substitution of an amino acid corresponding to position 52 with T; substitution of an amino acid corresponding to position 73 with S; substitution of an amino acid corresponding to position 78 with Y; substitution of an amino acid corresponding to position 132 with Y; substitution of an amino acid corresponding to position 161 with D; substitution of an amino acid corresponding to position 173 with S or K; substitution of an amino acid corresponding to position 213 with T; substitution of an amino acid corresponding to position 256 with Q or N; substitution of an amino acid corresponding to position 260 with V; and substitution of an amino acid corresponding to position 324 with D, provided that the position numberings are based on the amino acid positions within the reference amino acid sequence of SEQ ID NO: 1.

In another particular embodiment of the present disclosure, the amino acid substitutions in the amino acid sequence of SEQ ID NO: 4 may include one or more substitutions selected from a group consisting of: Q52T (substitution of glutamine [Q] with threonine [T] at position 52), S158D (substitution of serine [S] with aspartic acid [D] at position 158), Q170S (substitution of glutamine [Q] with serine [S] at position 170), S253Q (substitution of serine [S] with glutamine [Q] at position 253), and E321D (substitution of glutamic acid [E] with aspartic acid [D] at position 321), provided that the position numberings start from N-terminus of the amino acid sequence of SEQ ID NO: 4. More specifically, the amino acid substitutions may include 1, 2, 3, or 4 substitutions selected from the above group, or all five substitutions. A polypeptide including such amino acid substitutions provides an advantage of further enhanced structural stability.

In the present disclosure, the amino acid substitutions that significantly enhance structural stability the most in the amino acid sequence of SEQ ID NO: 4 are, in order: S253Q (substitution of serine with glutamine at position 253), Q170S (substitution of glutamine with serine at position 170), Q52T (substitution of glutamine with threonine at position 52), S158D (substitution of serine with aspartic acid at position 158), and E321D (substitution of glutamic acid with aspartic acid at position 321), provided that the position numberings start from N-terminus of the amino acid sequence of SEQ ID NO: 4.

In another particular embodiment of the present disclosure, the amino acid substitutions in the amino acid sequence of SEQ ID NO: 5 may include one or more substitutions selected from a group consisting of: S24E (substitution of serine with glutamic acid at position 24 from the N-terminus), S47M (substitution of serine with methionine at position 47), T70S (substitution of threonine with serine at position 70), R75Y (substitution of arginine with tyrosine at position 75), E129Y (substitution of glutamic acid with tyrosine at position 129), Q170K (substitution of glutamine with lysine at position 170), G210T (substitution of glycine with threonine at position 210), S253N (substitution of serine with asparagine at position 253), and A257V (substitution of alanine with valine at position 257), provided that the position numberings start from N-terminus of the amino acid sequence of SEQ ID NO: 5. More specifically, the amino acid substitutions may include 1, 2, 3, 4, 5, 6, 7, 8 or 9 substitutions selected from the above group, or all nine substitutions. A polypeptide including such amino acid substitutions provides an advantage of further enhanced structural stability.

In another embodiment of the present disclosure, a polynucleotide encoding a polypeptide for preventing or treating SARS-CoV-2 infection is provided.

In the present disclosure, the polynucleotide is a polymer of nucleotides in which nucleotide monomers are covalently linked in a long chain, and may be a DNA or RNA strand having a certain length, particularly an mRNA strand.

In a specific embodiment of the present disclosure, the polynucleotide encoding the polypeptide for preventing or treating SARS-CoV-2 infection may be an mRNA including the sequences of SEQ ID NOS: 6 to 9, as shown in Table 6.

TABLE 6
mRNA sequence corresponding to SEQ ID NO: 2 (Consensus_dsg;
Css_dsg) (5′ → 3′) (SEQ ID NO: 6)
AUGUUCGUGUUCCUUGUGCUGCUGCCUCUGGUCAGCUCUCAGUGCGUAAACCUGACAACCCGGA
CUCAGCUGCCUCCUGCCUACACCAACUCUUUCACCAGAGGCGUGUACUACCCUGACAAGGUGUU
CAGAAGCAGCGUGCUGCACAGCACACAGGAUCUGUUCCUGCCAUUUUUCAGCAACGUGACCUGG
UUCCACGCCAUAAGCGGCACCAACGGCACGAAGCGGUUUGAUAACCCCGUCCUCCCUUUCAACG
ACGGCGUAUACUUCGCCAGCACCGAGAAGAGCAACAUCAUCCGGGGAUGGAUCUUCGGUACAAC
GCUGGACAGCAAGACCCAGAGCCUGCUGAUCGUGAACAAUGCCACCAACGUGGUGAUUAAGGUG
UGCGAGUUCCAGUUCUGCAACGACCCUUUUCUGGGCGUGUACCACAAGAACAACAAGAGCUGGA
UGGAAAGCGAGUUCAGAGUGUACAGCAGCGCAAACAAUUGUACCUUUGAGUACGUGUCUCAGCC
UUUCCUGAUGGACCUGGAGGGCAAACAGGGCAAUUUCAAAAACCUGAGAGAGUUCGUGUUCAAG
AACAUCGACGGAUACUUCAAGAUCUACAGCAAGCACACACCGAUCAAUCUGGUGCGCGAUCUGC
CCCAGGGAUUUUCCGCUCUGGAACCCCUGGUGGACCUCCCUAUCGGCAUCAAUAUAACCCGGUU
UCAGACGCUGCUGGCCCUGCACAGAAGUUACCUGACCCCUGGCGAUUCCUCCAGCGGCUGGACC
GCCGGUGCCGCUGCCUACUACGUGGGCUACCUGCAGCCCCGCACCUUCCUGCUGAAGUACAAUG
AAAACGGAACCAUCACUGAUGCCGUGGACUGCGCCCUGGACCCUCUGUCCGAGACAAAGUGUAC
ACUGAAGAGCUUCACAGUGGAGAAGGGCAUCUAUCAGACCUCGAAUUUUAGAGUGCAGCCAACA
GAAAGCAUCGUGAGAUUUCCCAACAUCACCAACCUGUGCCCUUUUGGCGAGGUCUUCAACGCCA
CAAGAUUCGCCUCUGUAUAUGCCUGGAACAGAAAGAGAAUCUCUAACUGCGUGGCAGACUACUC
GGUUCUGUACAACUCAGCCUCUUUUUCCACAUUCAAGUGCUACGGCGUGUCUCCUACGAAGCUG
AACGACCUGUGCUUCACAAACGUGUACGCCGACAGCUUCGUGAUCAGAGGAGAUGAGGUGAGAC
AGAUCGCCCCAGGCCAGACAGGCAAGAUCGCCGACUACAAUUAUAAGCUGCCCGAUGAUUUCAC
AGGGUGCGUGAUCGCCUGGAACUCAAAUAACCUCGAUAGCAAGGUGGGCGGAAAUUACAACUAC
CGCUACAGACUGUUUCGGAAGAGUAACCUUAAACCUUUCGAGAGAGACAUCUCCACCGAGAUCU
ACCAGGCCGGAUCUAAGCCUUGCAAUGGCGUGGAAGGAUUCAACUGCUACUUCCCUCUGCAGUC
UUACGGCUUUCAGCCUACAAAUGGAGUGGGCUACCAGCCCUACCGUGUGGUGGUCCUGAGCUUU
GAGCUGCUGCAUGCUCCUGCCACCGUCUGCGGCCCUAAGAAGUCUACCAACCUGGUGAAGAACA
AAUGCGUGAACUUCAAUUUCAACGGGCUGACCGGCACAGGCGUGCUGACCGAAUCUAACAAGAA
AUUCCUGCCCUUCCAGCAGUUCGGCCGGGACAUUGCCGACACCACAGACGCCGUGCGGGAUCCC
CAGACUCUGGAAAUCCUGGACAUCACCCCUUGCAGCUUCGGCGGCGUGAGCGUGAUUACCCCAG
GCACCAACACAUCUAAUCAGGUUGCCGUGCUGUACCAGGGCGUUAACUGCACAGAAGUGCCUGU
CGCCAUCCACGCCGACCAGCUGACCCCUACGUGGCGGGUUUACAGCACCGGCAGCAACGUGUUC
CAGACUAGAGCCGGCUGCCUGAUCGGCGCCGAGCACGUCAACAAUAGCUACGAGUGUGACAUUC
CUAUCGGCGCCGGCAUCUGCGCCUCUUACCAGACCCAGACCAACAGCCCAGCA
mRNA sequence corresponding to SEQ ID NO: 3 (Chimeric_dsg;
Chi_dsg) (5′ → 3′) (SEQ ID NO: 7)
AUGUUCGUGUUCCUGGUGCUGCUGCCUCUGGUGAGCAGCCAGUGCGUGAAUCUGAUUACAAGAA
CUCAGGAGUACACUAACUCUUUCACCAGAGGAGUGUACUACCCCGAUAAGGUGUUUAGGUCAAG
CGUGCUGCACAUGACUCAGGACCUGUUCCUGCCUUUCUUUUCUAAUGUGACCUGGUUCCACGCA
AUCCACGUGAGCGGCAGCAACGGCACGAAAUAUUUCGAUAAUCCUGUGCUGCCCUUCAACGACG
GCGUGUACUUCGCCUCCACAGAGAAGUCAAACAUUAUUCGGGGGUGGAUCUUCGGCACCACACU
GGACAGUAAAACCCAGUCCCUGCUGAUCGUGAACAAUGCUACCAACGUGGUGAUUAAGGUGUGC
UACUUUCAGUUCUGCAAUGAUCCAUUCCUGGAUGUGUACUACCACAAGAACAAUAAGUCCUGGA
UGGAAAGCGAAUUCAGAGUGUAUUCCUCUGCAAAUAACUGUACAUUCGAGUACGUGUCCAAGCC
AUUCCUCAUGGACCUUGAAGGCAAGCAGGGGAAUUUUAAAAACCUGCGGGAGUUUGUGUUUAAG
AAUAUCGAUGGCUACUUCAAGAUUUAUUCCAAACACACUCCCAUCAACCUGACCAGGGACCUGC
CCCAGGGCUUUUCCGCCCUGGAGCCCCUGGUGGACCUGCCCAUUGGCAUUAACAUUACAAGAUU
CCAGACCCUGCUGGCUCUGCACAGGUCCUAUCUGACACCUGGCGAUAGCAGCAAUGGCUGGACC
GUCGGGGCAGCCGCCUAUUACGUGGGAUACCUGCAGCCCCGGACCUUUCUGCUGAAGUACAAUG
AGAACGGCACAAUCACAGACGCUGUGGACUGCGCCCUGGACCCACUGAGCGAGACUAAGUGCAC
UCUGAAAUCAUUCACUGUGGAGAAGGGGAUUUAUCAGACAUCUAACUUCAGAGUCCAGCCUACC
GAGAGUAUCGUGAGGUUCCCCAACAUCACCAACCUGUGCCCUUUUCACGAGGUGUUUAACGCCA
CCACCUUCGCCUCAGUGUAUGCCUGGAACAGGAAGAGAAUCAGCAAUUGCGUGGCAGACUACUC
CGUGCUGUACAACUUCGCACCCUUCUUCGCCUUUAAGUGUUACGGCGUGAGCCCCACCAAACUG
AACGAUCUGUGCUUCACAAAUGUGUACGCCGAUAGUUUUGUGAUCCGGGGGAAUGAGGUGUCUC
AAAUCGCCCCCGGCCAGACCGGAAACAUCGCUGAUUAUAACUACAAGCUGCCCGAUGAUUUUAC
CGGGUGCGUGAUCGCCUGGAAUAGUAAUAAGCUGGAUAGCAAAGUGUCUGGCAACUAUAACUAU
CUGUACCGGCUGUUCAGGAAAUCCAAGCUGAAGCCCUUCGAGCGCGACAUCUCUACCGAAAUUU
ACCAGGCCGGUAACAAGCCAUGUAACGGCGUGGCUGGCUUUAAUUGCUACUUCCCACUGCAGAG
CUACGGGUUCCGGCCCACCUAUGGGGUGGGGCAUCAGCCCUACCGCGUGGUGGUGCUGUCCUUC
GAGCUGCUGCACGCCCCAGCUACCGUGUGCGGGCCCAAGAAGUCAACUAAUCUGGUCAAGAACA
AGUGUGUCAACUUCAACUUCAAUGGCCUGACUGGGACAGGCGUGCUCACCGAGAGCAACAAGAA
AUUCCUGCCUUUUCAGCAGUUCGGCAGGGACAUCGCCGACACCACAGAUGCCGUUCGGGACCCA
CAGACCCUGGAAAUCCUGGAUAUUACUCCAUGCAGCUUCGGCGGCGUGUCCGUGAUUACACCUG
GAACCAACACCAGCAACCAGGUGGCCGUGCUGUACCAGGGCGUGAACUGCACCGAGGUGCCUGU
GGCAAUACACGCCGACCAGCUGACUCCCACAUGGCGAGUGUAUUCAACCGGCUCCAAUGUGUUU
CAGACUAGAGCCGGCUGCCUGAUCGGAGCUGAGCACGUGAAUAACUCUUAUGAGUGCGAUAUCC
CCAUCGGAGCCGGCAUCUGCGCAUCCUAUCAGACACAGACAAACAGCCACGCC
mRNA sequence corresponding to SEQ ID NO: 4 (Consensus; Css)
(5′ → 3′) (SEQ ID NO: 8)
AUGUUCGUGUUCCUUGUGCUGCUGCCUCUGGUCAGCUCUCAGUGCGUAAACCUGACAACCCGGA
CUCAGCUGCCUCCUGCCUACACCAACUCUUUCACCAGAGGCGUGUACUACCCUGACAAGGUGUU
CAGAAGCAGCGUGCUGCACAGCACACAGGAUCUGUUCCUGCCAUUUUUCAGCAACGUGACCUGG
UUCCACGCCAUAAGCGGCACCAACGGCACGAAGCGGUUUGAUAACCCCGUCCUCCCUUUCAACG
ACGGCGUAUACUUCGCCAGCACCGAGAAGAGCAACAUCAUCCGGGGAUGGAUCUUCGGUACAAC
GCUGGACAGCAAGACCCAGAGCCUGCUGAUCGUGAACAAUGCCACCAACGUGGUGAUUAAGGUG
UGCGAGUUCCAGUUCUGCAACGACCCUUUUCUGGGCGUGUACCACAAGAACAACAAGAGCUGGA
UGGAAAGCGAGUUCAGAGUGUACAGCAGCGCAAACAAUUGUACCUUUGAGUACGUGUCUCAGCC
UUUCCUGAUGGACCUGGAGGGCAAACAGGGCAAUUUCAAAAACCUGAGAGAGUUCGUGUUCAAG
AACAUCGACGGAUACUUCAAGAUCUACAGCAAGCACACACCGAUCAAUCUGGUGCGCGAUCUGC
CCCAGGGAUUUUCCGCUCUGGAACCCCUGGUGGACCUCCCUAUCGGCAUCAAUAUAACCCGGUU
UCAGACGCUGCUGGCCCUGCACAGAAGUUACCUGACCCCUGGCGAUUCCUCCAGCGGCUGGACC
GCCGGUGCCGCUGCCUACUACGUGGGCUACCUGCAGCCCCGCACCUUCCUGCUGAAGUACAAUG
AAAACGGAACCAUCACUGAUGCCGUGGACUGCGCCCUGGACCCUCUGUCCGAGACAAAGUGUAC
ACUGAAGAGCUUCACAGUGGAGAAGGGCAUCUAUCAGACCUCGAAUUUUAGAGUGCAGCCAACA
GAAAGCAUCGUGAGAUUUCCCAACAUCACCAACCUGUGCCCUUUUGGCGAGGUCUUCAACGCCA
CAAGAUUCGCCUCUGUAUAUGCCUGGAACAGAAAGAGAAUCUCUAACUGCGUGGCAGACUACUC
GGUUCUGUACAACUCAGCCUCUUUUUCCACAUUCAAGUGCUACGGCGUGUCUCCUACGAAGCUG
AACGACCUGUGCUUCACAAACGUGUACGCCGACAGCUUCGUGAUCAGAGGAGAUGAGGUGAGAC
AGAUCGCCCCAGGCCAGACAGGCAAGAUCGCCGACUACAAUUAUAAGCUGCCCGAUGAUUUCAC
AGGGUGCGUGAUCGCCUGGAACUCAAAUAACCUCGAUAGCAAGGUGGGCGGAAAUUACAACUAC
CGCUACAGACUGUUUCGGAAGAGUAACCUUAAACCUUUCGAGAGAGACAUCUCCACCGAGAUCU
ACCAGGCCGGAUCUAAGCCUUGCAAUGGCGUGGAAGGAUUCAACUGCUACUUCCCUCUGCAGUC
UUACGGCUUUCAGCCUACAAAUGGAGUGGGCUACCAGCCCUACCGUGUGGUGGUCCUGAGCUUU
GAGCUGCUGCAUGCUCCUGCCACCGUCUGCGGCCCUAAGAAGUCUACCAACCUGGUGAAGAACA
AAUGCGUGAACUUCAAUUUCAACGGGCUGACCGGCACAGGCGUGCUGACCGAAUCUAACAAGAA
AUUCCUGCCCUUCCAGCAGUUCGGCCGGGACAUUGCCGACACCACAGACGCCGUGCGGGAUCCC
CAGACUCUGGAAAUCCUGGACAUCACCCCUUGCAGCUUCGGCGGCGUGAGCGUGAUUACCCCAG
GCACCAACACAUCUAAUCAGGUUGCCGUGCUGUACCAGGGCGUUAACUGCACAGAAGUGCCUGU
CGCCAUCCACGCCGACCAGCUGACCCCUACGUGGCGGGUUUACAGCACCGGCAGCAACGUGUUC
CAGACUAGAGCCGGCUGCCUGAUCGGCGCCGAGCACGUCAACAAUAGCUACGAGUGUGACAUUC
CUAUCGGCGCCGGCAUCUGCGCCUCUUACCAGACCCAGACCAACAGCCCAGCA
mRNA sequence corresponding to SEQ ID NO: 5 (Chimeric; Chi)
(5′ → 3′) (SEQ ID NO: 9)
AUGUUUGUGUUUCUGGUUCUCCUCCCCCUGGUCAGUAGCCAGUGCGUAAAUCUGAUCACUCGCA
CACAAUCAUACACGAAUUCUUUCACACGCGGGGUCUAUUAUCCUGAUAAGGUUUUUAGAAGUUC
UGUACUCCACUCUACACAAGACCUGUUCCUGCCGUUUUUCUCCAACGUAACAUGGUUUCACGCG
AUUCAUGUCUCCGGAACGAACGGGACAAAACGGUUUGAUAAUCCUGUUCUCCCUUUUAAUGACG
GGGUUUAUUUCGCAAGCACAGAGAAGUCAAACAUUAUUAGGGGGUGGAUCUUUGGUACCACUUU
GGAUUCCAAAACACAAAGCCUGCUUAUAGUGAACAAUGCGACCAAUGUGGUAAUAAAAGUCUGU
GAAUUCCAGUUCUGCAAUGAUCCUUUCCUUGAUGUCUAUUACCACAAGAACAACAAGAGCUGGA
UGGAGUCCGAAUUCCGGGUGUAUAGCAGCGCUAACAAUUGUACGUUUGAGUACGUCUCUCAACC
UUUCUUGAUGGACCUGGAGGGUAAACAGGGGAACUUCAAAAACCUUCGGGAAUUCGUUUUCAAG
AAUAUUGACGGCUACUUCAAAAUAUACAGUAAACAUACUCCCAUUAACCUCGGACGCGACCUCC
CACAAGGCUUCAGCGCGCUUGAACCACUCGUAGACCUCCCUAUCGGAAUCAAUAUCACCCGCUU
CCAAACUCUCCUGGCCCUCCACCGGUCAUAUCUGACUCCCGGUGAUUCUAGCUCUGGUUGGACA
GCAGGGGCAGCCGCAUACUAUGUCGGGUAUCUUCAACCUCGCACUUUCCUGCUUAAGUACAACG
AAAAUGGCACUAUUACCGACGCUGUAGAUUGCGCUCUUGACCCGUUGAGUGAGACGAAGUGCAC
CCUCAAGAGCUUUACCGUCGAGAAGGGGAUCUACCAGACGUCAAAUUUUAGAGUCCAACCGACG
GAGUCUAUCGUCCGGUUUCCUAACAUUACUAACCUGUGUCCAUUUCAUGAAGUGUUCAAUGCAA
CGACCUUCGCAUCUGUGUAUGCUUGGAAUCGCAAGAGGAUUAGUAAUUGCGUUGCUGAUUACUC
CGUUCUUUACAACUUCGCCCCGUUCUUCGCUUUUAAGUGCUACGGUGUUAGUCCAACUAAACUG
AACGACCUUUGCUUCACUAAUGUUUACGCAGAUUCAUUUGUCAUUCGGGGCAAUGAAGUCAGCC
AGAUCGCCCCUGGCCAAACGGGGAAUAUAGCUGACUACAAUUACAAGCUGCCCGACGACUUUAC
GGGGUGUGUCAUUGCGUGGAACAGUAAUAAACUCGACUCAAAGGUUAGUGGUAACUAUAAUUAU
CUUUAUCGGUUGUUUAGAAAGAGUAAAUUGAAGCCUUUUGAAAGGGAUAUUUCAACAGAGAUAU
AUCAAGCGGGUAAUAAGCCAUGCAACGGCGUGGCGGGCUUUAACUGUUACUUUCCUCUGCAAUC
CUACGGCUUUAGACCAACAUAUGGAGUCGGGCAUCAACCCUAUCGGGUUGUGGUUUUGUCUUUC
GAGCUGCUCCACGCACCGGCCACUGUAUGCGGCCCCAAAAAAAGUACCAAUUUGGUGAAAAAUA
AAUGUGUGAAUUUUAAUUUUAACGGGUUGACAGGAACUGGGGUACUCACUGAAUCUAAUAAAAA
AUUCCUCCCAUUUCAACAAUUCGGGAGAGAUAUAGCAGAUACAACAGAUGCCGUGCGGGACCCC
CAGACGUUGGAAAUCCUUGAUAUCACGCCAUGUUCCUUCGGCGGGGUAAGCGUUAUAACCCCCG
GGACGAACACCAGUAAUCAAGUUGCGGUCCUCUAUCAGGGUGUUAACUGUACGGAAGUUCCUGU
GGCCAUCCACGCUGACCAACUGACGCCAACUUGGAGAGUAUACAGCACUGGCUCCAAUGUCUUU
CAGACACGCGCGGGCUGUCUUAUUGGUGCUGAGCAUGUCAAUAACAGUUACGAGUGCGAUAUCC
CGAUAGGAGCUGGCAUAUGUGCCUCAUACCAAACCCAGACGAAUUCUCAUGCC

In the present disclosure, the term “SARS-CoV-2 infection” is understood to broadly encompass not only infection by the SARS-CoV-2 virus itself, but also various secondary diseases resulting from such infection, including but not limited to respiratory diseases and pneumonia.

In the present disclosure, the SARS-CoV-2 includes not only the wild-type SARS-CoV-2 S strain (Wuhan strain), but also all variants of SARS-CoV-2.

SARS-CoV-2 is an RNA virus known to evolve with a high mutation frequency during genome replication. Generally, SARS-CoV-2 forms lineages, which are groups of genetically related viral variants derived from a common ancestor. Since the onset of pandemic, SARS-CoV-2 has continuously mutated to form such lineages. These lineages are classified and managed based on factors including mutation frequency, geographic distribution, impacts of mutation clusters on medical countermeasures, disease severity, and known or potential effects on human-to-human transmissibility.

The World Health Organization (WHO) classifies SARS-CoV-2 variants, based on their public health risk, into three categories: Variant of Concern (VOC), Variant of Interest (VOI), and Variant Under Monitoring (VUM). The occurrence and public health impact of each variant are assessed and periodically updated by WHO. The “Variant of Concern (VOC)” refers to a variant for which negative changes in transmissibility, pathogenicity, or vaccine effectiveness have been confirmed, thereby requiring public health interventions. The “Variant of Interest (VOI)” refers to a variant that possesses amino acid mutations different from those of a reference strain, and has demonstrated community transmission, or been detected in multiple countries, but has not yet been confirmed to pose clinical or epidemiological risk. Variants, in which have been identified amino acid mutations, but have insufficient evidence related to phenotypic changes, are classified as a “Variants Under Monitoring (VUM)”.

In the above classification of SARS-CoV-2 variants, Alpha, Beta, Gamma, Delta, and Omicron variants are classified into the VOC.

In the present disclosure, the SARS-CoV-2 variants are not particularly limited to types, and are understood to encompass both currently existing variants, and potential future variants that may emerge. Non-limiting examples of the SARS-CoV-2 variants include Alpha, Beta, Gamma, Delta, and Omicron variants.

In another embodiment of the present disclosure, a vaccine composition for preventing SARS-CoV-2 infection is provided, wherein the vaccine composition contains the polypeptide for preventing or treating SARS-CoV-2 infection, as an active ingredient.

In another embodiment of the present disclosure, a pharmaceutical composition for preventing or treating infection is provided, wherein the pharmaceutical composition contains the polypeptide for preventing or treating SARS-CoV-2 infection, as an active ingredient.

In the present disclosure, the vaccine composition or pharmaceutical composition for preventing or treating SARS-CoV-2 infection may be formulated using the polypeptide for preventing or treating SARS-CoV-2 infection described herein as an antigen protein. Alternatively, the vaccine or pharmaceutical composition may be formulated to include other SARS-CoV-2-related antigen protein domains, in addition to the polypeptide of the present disclosure. The specific types of SARS-CoV-2-related antigen protein domains that may be included together with the polypeptide of the present disclosure, are not particularly limited, but for exerting superior antigenicity, such domains may include those constituting a SARS-CoV-2 spike(S) protein, and more specifically, S2 domain. In more particular, the polypeptide of the present disclosure may be configured as a full-length S protein linked to the S2 domain including 6P (K986P, V987P, F817P, A892P, A899P, A942P) that stabilize a prefusion structure of the S protein, and may be used as an active ingredient in the vaccine or pharmaceutical composition. Specifically, a S2 subunit may include the D614G substitution, in which aspartic acid at position 614 of a wild-type SARS-CoV-2 spike protein is replaced with glycine. The S2 subunit of selected universal spike protein may further include HexaPro mutations (F817P, A892P, A899P, A942P, K986P, V987P) for protein stabilization, as described in Hsieh et al., Science 369:1501-05 (2020), as well as mutations in S1/S2 cleavage site (682RRAR685 to A).

The SARS-CoV-2-related antigen proteins that may be included together with the polypeptide of the present disclosure are understood to encompass not only antigen proteins of a wild-type SARS-CoV-2 S strain (Wuhan strain), but also those of SARS-CoV-2 variants, such as the Alpha, Beta, Gamma, Delta, and Omicron variants.

In another embodiment of the present disclosure, a vaccine composition for preventing SARS-CoV-2 infection is provided, wherein the vaccine composition contains a polynucleotide encoding a polypeptide for preventing or treating SARS-CoV-2 infection as described herein, as an active ingredient.

In another embodiment of the present disclosure, a pharmaceutical composition for preventing or treating SARS-CoV-2 infection is provided, wherein the pharmaceutical composition contains a polynucleotide encoding a polypeptide for preventing or treating SARS-CoV-2 infection as described herein, as an active ingredient.

In the present disclosure, the vaccine or pharmaceutical composition for preventing or treating SARS-CoV-2 infection may be formulated using the polynucleotide for preventing or treating SARS-CoV-2 infection, as described herein. In addition, the vaccine or pharmaceutical composition may be formulated to include polynucleotides encoding other SARS-CoV-2-related antigen protein domains, in addition to the polynucleotide of the present disclosure. The specific types of SARS-CoV-2-related antigen protein domains encoded by polynucleotides which are included together with the polynucleotide of the present disclosure, are not particularly limited, but for exerting superior antigenicity, such domains may include those constituting the SARS-CoV-2 spike(S) protein, and more specifically, a S2 domain. In more particular, the vaccine or pharmaceutical composition utilizing the polynucleotide of the present disclosure may include a polynucleotide encoding a full-length S protein, in which the S2 domain including HexaPro mutations (K986P, V987P, F817P, A892P, A899P, A942P), is linked to the polypeptide of the present disclosure, as an active ingredient.

The SARS-CoV-2-related antigen proteins that may be encoded by polynucleotides that may be included together with the polynucleotide of the present disclosure, are understood to encompass not only the antigen proteins of a wild-type SARS-CoV-2 S strain (Wuhan strain), but also those of SARS-CoV-2 variants, such as Alpha, Beta, Gamma, Delta, and Omicron variants.

In the present disclosure, the “vaccine” refers to a biological composition containing an antigen capable of eliciting an immune response in the body, and may be used as an immunogen that induces immunity in the body when administered, for example, via injection or oral administration, for preventing infection.

The vaccine composition of the present disclosure may further contain liposomes or lipid nanoparticles (LNPs). The polynucleotide contained in the vaccine composition as an active ingredient, particularly mRNA, may be adsorbed or associated on exteriors of the liposomes or lipid nanoparticles, or may be encapsulated within them. In the present disclosure, the liposomes or lipid nanoparticles may contain cationic lipids, and preferably may additionally contain neutral lipids.

In the present disclosure, by using conventional methods, the vaccine composition may be formulated into various oral dosage forms, such as powders, granules, tablets, capsules, suspensions, emulsions, syrups, and aerosols, as well as into sterile injectable solutions. During formulation, commonly used diluents or excipients, such as fillers, bulking agents, binders, wetting agents, disintegrants, and surfactants, may be employed.

Solid oral dosage forms may include tablets, pills, powders, granules, and capsules. These solid oral dosage forms may be prepared by mixing lecithin-like emulsifier with at least one excipient, such as starch, calcium carbonate, sucrose, lactose, or gelatin. In addition to simple excipients, lubricants such as magnesium stearate and talc may also be used. Liquid oral dosage forms may include suspensions, internal solutions, emulsions, and syrups. In addition to commonly used diluents such as water or liquid paraffin, various excipients including wetting agents, sweeteners, flavoring agents, and preservatives may be included. Non-oral dosage forms may include sterile aqueous solutions, non-aqueous formulations, suspensions, emulsions, and lyophilized preparations. Non-aqueous agents, injectable esters such as ethyl oleate, or plant-based oils such as olive oil, as well as suspending agent such as propylene glycol and polyethylene glycol may also be used.

In the present disclosure, the administration routes of the vaccine composition may include oral, intravenous, intramuscular, intra-arterial, intramedullary, intradural, intracardiac, transdermal, subcutaneous, intraperitoneal, intranasal, enteral, topical, sublingual, or rectal routes, but not limited thereto. Oral or parenteral administration is preferred. As used herein, the term “parenteral” includes injection or infusion techniques such as subcutaneous, intradermal, intravenous, intramuscular, intra-articular, intra-bursal, intra-sternal, intradural, intralesional, and intracranial injection or infusion techniques. The vaccine composition of the present disclosure may also be administered in the form of suppositories for rectal delivery.

The dosage of the vaccine composition according to the present disclosure may be readily determined by those skilled in the art within a wide range, considering factors such as intended use, types of target diseases, age, weight, and medical history of a subject (or a patient). Likewise, the administration frequency may also be readily determined by those skilled in the art, considering intended use, type and severity of a target disease, age, weight, medical history, and clinical progress of a subject (or a patient).

The vaccine composition of the present disclosure may further contain an adjuvant depending on intended use. As used herein, the term “adjuvant” refers to a substance or composition that is added to a vaccine or pharmacologically active ingredient to enhance and/or affect an immune response. The adjuvant may include a wide range of substances or immunostimulant capable of enhancing an immunogenicity of an antigen that is either integrated into or co-administered with the adjuvant.

The adjuvants that may be used together with the vaccine composition of the present disclosure may include aluminum hydroxide, aluminum phosphate, alum (potassium aluminum sulfate), MF59, virosomes, AS04 (a mixture of aluminum hydroxide and monophosphoryl lipid A [MPL]), AS03 (a mixture of DL-α-tocopherol, squalene, and the emulsifier polysorbate 80), CpG, flagellin, poly I:C, AS01, AS02, ISCOMs, and ISCOMMATRIX, but are not limited thereto.

The adjuvant composition of the present disclosure may be administered concurrently with the vaccine or at a different time. The administration frequency of the adjuvant composition may vary, for example, from daily to once every few months, or once or twice prior to each epidemic season, but not limited thereto. Furthermore, the interval for additional vaccination may be readily determined by those skilled in the art, based on the maintenance duration of immunogenicity.

The pharmaceutical composition of the present disclosure may further contain suitable carriers, excipients, and diluents commonly used in the preparation of pharmaceutical compositions. The excipients may include one or more selected from a group consisting of diluents, binders, disintegrants, glidants, adsorbents, moisturizers, film-coating agents, and controlled-release additives.

By using conventional methods, the pharmaceutical composition according to the present disclosure may be formulated into various external preparations, including powders, granules, sustained-release granules, enteric-coated granules, liquids, eye drops, elixirs, emulsions, suspensions, tinctures, aromatic lozenges, waters, lemonades, tablets, sustained-release tablets, enteric-coated tablets, sublingual tablets, hard capsules, soft capsules, sustained-release capsules, enteric-coated capsules, pills, tinctures, soft extracts, dry extracts, fluid extracts, injections, capsules, perfusion fluids, ointments, lotions, pastes, sprays, inhalants, patches, sterile injectable solutions, and aerosols. The external preparations may be formulated as creams, gels, patches, sprays, ointments, counterirritant, lotions, liniments, pastes, or cataplasms.

Carriers, excipients, and diluents that may be included in the pharmaceutical composition of the present disclosure, may include lactose, dextrose, sucrose, oligosaccharides, sorbitol, mannitol, xylitol, erythritol, maltitol, starch, gum arabic, alginate, gelatin, calcium phosphate, calcium silicate, cellulose, methylcellulose, microcrystalline cellulose, polyvinylpyrrolidone, water, methylhydroxy benzoate, propylhydroxy benzoate, talc, magnesium stearate, and mineral oil.

The pharmaceutical composition of the present disclosure is administered in a pharmaceutically effective amount. As used herein, the term “pharmaceutically effective amount” refers to an amount sufficient to treat a disease with a reasonable benefit/risk ratio applicable to medical treatment. The effective dosage level may be determined based on factors such as type and severity of the disease, activity of a drug, sensitivity to a drug, administration timing, route of administration, elimination rate, treatment duration, concomitant medications, and other factors well known in the medical field.

The pharmaceutical composition of the present disclosure may be administered as a monotherapy, or in combination with other therapeutic agents either sequentially or simultaneously, and may be administered in single or multiple doses. It is important to administer an amount that, even when administered at a minimal dose, achieves maximum efficacy without side effects, considering factors aforementioned, and such dosage may be readily determined by those skilled in the art.

The pharmaceutical composition of the present disclosure may be administered to a subject via various routes. All administration routes may be contemplated, including oral administration, subcutaneous injection, intraperitoneal administration, intravenous injection, intramuscular injection, perispinal injection, intrathecal injection, sublingual administration, buccal administration, rectal insertion, vaginal insertion, ocular administration, auricular administration, nasal administration, inhalation, spraying through the mouth or nose, intradermal administration, and transdermal administration.

Unless otherwise defined herein, the terms used in the present disclosure shall be interpreted as having meanings commonly understood in the relevant technical field. Furthermore, the term “or” as used in the present disclosure shall be interpreted to include “and” unless otherwise specified.

The scope of the present disclosure is not limited by the specific descriptions set forth herein. Each description and embodiment disclosed herein may be applied to other descriptions and embodiments. That is, all possible combinations of the various elements disclosed herein shall be construed as falling within the scope of the present disclosure. Moreover, those skilled in the art may recognize or derive numerous equivalents to specific embodiments of the present disclosure through routine experimentation, and such equivalents shall be construed as falling within the scope of the present disclosure.

The present disclosure relates to a polypeptide including a SARS-CoV-2 universal antigen amino acid sequence, and a polynucleotide encoding the polypeptide, as well as a vaccine composition for preventing SARS-CoV-2 infection, and a pharmaceutical composition for preventing or treating SARS-CoV-2 infection, wherein the vaccine and pharmaceutical composition contains the polypeptide or polynucleotide as an active ingredient.

The present disclosure was completed by analyzing approximately 2,000 sequences including all existing genotypes of SARS-CoV-2 variants, specifically S1 subunits of the SARS-CoV-2 spike proteins which serve as a major antigen of the virus, thereby deriving a conserved consensus sequence to obtain a SARS-CoV-2 universal antigen amino acid sequence. This provides an advantage of offering a universal antigen polypeptide capable of responding not only to a wild-type SARS-CoV-2 and existing variants, but also to potential future variants.

Furthermore, to enhance structural stability of the SARS-CoV-2 universal antigen polypeptide, the present disclosure offers an advantage of providing an optimized SARS-CoV-2 universal antigen amino acid sequence produced by substituting specific amino acids at designated positions within amino acids of the universal antigen polypeptide, and the universal polypeptide synthesized from the amino acid sequence.

Specifically, it was experimentally confirmed that the amino acid substitutions at positions 173 (Q173) and 256 (S256) of the spike protein function as universal stabilization sites that enhance protein expression and stability. In particular, when the double mutations Q173S and S256Q were introduced, the most significant increase in expression and stabilization effects was observed. These effects were consistently verified in Omicron subvariants, including the JN.1 variant, demonstrating that the stabilization design of the present disclosure is not limited to a specific variant but can be universally applied across SARS-CoV-2 variants.

The present disclosure also provides a polynucleotide encoding the SARS-CoV-2 universal antigen polypeptide, and by utilizing the SARS-CoV-2 universal antigen polypeptide or polynucleotide used in vaccine or pharmaceutical compositions, the present disclosure offers an advantage of providing the vaccine or pharmaceutical compositions capable of responding to wild-type SARS-CoV-2, existing variants, and potential future variants for preventing or treating SARS-CoV-2 infection.

### BRIEF DESCRIPTION OF THE DRAWINGS

The above and other objectives, features and other advantages of the present disclosure will be more clearly understood from the following detailed description taken in conjunction with the accompanying drawings.

FIG. 1A illustrates amino acid substitution positions derived by using SEQ ID NO: 1 as a reference sequence for designing a SARS-CoV-2 universal antigen polypeptide of SEQ ID NO: 2, mapped onto a three-dimensional protein structure, according to one embodiment of the present disclosure. The green-colored region represents the NTD (N-terminal domain) of the S1 subunit, and the pink-colored region represents the RBD (Receptor Binding Domain). Each substituted amino acid position is labeled on the structure.

FIG. 1B illustrates a linear schematic diagram showing amino acid substitution positions in the SARS-CoV-2 universal antigen polypeptides of SEQ ID NOS: 2 and 3, according to one embodiment of the present disclosure. Among the two domains (NTD and RBD) of the S1 subunit, five substitution positions indicated in olive and red represent positions substituted in SEQ ID NO: 2, and nine substitution positions indicated in sky blue and red represent positions substituted in SEQ ID NO: 3. Common amino acid substitution positions in SEQ ID NOS: 2 and 3 are indicated in red.

FIG. 2 shows results of a Western blot experiment confirming universality of a SARS-CoV-2 universal antigen polypeptide (SEQ ID NO: 4) according to one embodiment of the present disclosure.

FIG. 3 is a schematic diagram of an animal vaccination experiment conducted to confirm immunogenicity of a SARS-CoV-2 universal mRNA vaccine according to one embodiment of the present disclosure.

FIG. 4A shows results of neutralizing antibody titer (ND₅₀) measurement against the SARS-CoV-2 Wuhan (wild-type) strain induced by the SARS-CoV-2 universal mRNA vaccine (SEQ ID NO: 12), according to one embodiment of the present disclosure.

FIG. 4B shows results of neutralizing antibody titer (ND₅₀) measurement against the SARS-CoV-2 Delta variant induced by the SARS-CoV-2 universal mRNA vaccine (SEQ ID NO: 12), according to one embodiment of the present disclosure.

FIG. 4C shows results of neutralizing antibody titer (ND₅₀) measurement against the SARS-CoV-2 Omicron BA. 5 variant induced by the SARS-CoV-2 universal mRNA vaccine (SEQ ID NO: 12), according to one embodiment of the present disclosure.

FIG. 5 shows results of IFN-γ induction by a SARS-CoV-2 universal mRNA vaccine (SEQ ID NO: 12) according to one embodiment of the present disclosure.

FIG. 6A shows results of body weight change analysis in mice infected with the SARS-CoV-2 Wuhan strain after vaccination with the SARS-CoV-2 universal mRNA vaccine (SEQ ID NO: 12), according to one embodiment of the present disclosure.

FIG. 6B shows results of body weight change analysis in mice infected with the SARS-CoV-2 Delta variant after vaccination with the SARS-CoV-2 universal mRNA vaccine (SEQ ID NO: 12), according to one embodiment of the present disclosure.

FIG. 6C shows results of survival rate analysis in mice infected with the SARS-CoV-2 Wuhan strain after vaccination with the SARS-CoV-2 universal mRNA vaccine (SEQ ID NO: 12), according to one embodiment of the present disclosure.

FIG. 6D shows results of survival rate analysis in mice infected with the SARS-CoV-2 Delta variant after vaccination with the SARS-CoV-2 universal mRNA vaccine (SEQ ID NO: 12), according to one embodiment of the present disclosure.

FIG. 7A shows results of viral titer measurement in lung tissues of mice infected with the SARS-CoV-2 Omicron BA.5 variant after vaccination with the SARS-CoV-2 universal mRNA vaccine (SEQ ID NO: 12), according to one embodiment of the present disclosure.

FIG. 7B shows results of viral genome titer measurement in lung tissues of mice infected with the SARS-CoV-2 Omicron BA. 5 variant after vaccination with the SARS-CoV-2 universal mRNA vaccine (SEQ ID NO: 12), using qPCR analysis, according to one embodiment of the present disclosure.

FIG. 8A shows results of immunogenicity evaluation of the SARS-COV-2 universal mRNA vaccine (SEQ ID NO: 12), representing neutralizing antibody titers (ND₅₀) against the SARS-CoV-2 Wuhan strain, according to one embodiment of the present disclosure.

FIG. 8B shows results of immunogenicity evaluation of the SARS-CoV-2 universal mRNA vaccine (SEQ ID NO: 12), representing neutralizing antibody titers (ND₅₀) against the SARS-CoV-2 Delta variant, according to one embodiment of the present disclosure.

FIG. 8C shows results of immunogenicity evaluation of the SARS-CoV-2 universal mRNA vaccine (SEQ ID NO: 12), representing neutralizing antibody titers (ND₅₀) against the SARS-CoV-2 Omicron BA.5 variant, according to one embodiment of the present disclosure.

FIG. 9A shows results of neutralizing antibody titer (ND₅₀) measurement against the SARS-CoV-2 Wuhan strain following administration of the SARS-CoV-2 universal mRNA vaccine (SEQ ID NO: 13), according to one embodiment of the present disclosure.

FIG. 9B shows results of neutralizing antibody titer (ND₅₀) measurement against the SARS-CoV-2 Omicron BA. 5 variant following administration of the SARS-CoV-2 universal mRNA vaccine (SEQ ID NO: 13), according to one embodiment of the present disclosure.

FIG. 9C shows results of neutralizing antibody titer (ND₅₀) measurement against the SARS-CoV-2 Omicron BN.1 variant following administration of the SARS-CoV-2 universal mRNA vaccine (SEQ ID NO: 13), according to one embodiment of the present disclosure.

FIG. 10 shows results of IFN-γ induction by a SARS-CoV-2 universal mRNA vaccine (SEQ ID NO: 13) according to one embodiment of the present disclosure.

FIG. 11 shows results verifying the universality of common mutation positions in the SARS-CoV-2 universal antigen sequences (SEQ ID NOS: 2 and 3), according to one embodiment of the present disclosure.

### DESCRIPTION OF THE PREFERRED EMBODIMENTS

Hereinafter, the present disclosure will be described in further detail through specific embodiments. However, these embodiments are provided solely for illustrative purposes and should not be construed as limiting the scope of the present disclosure in any way.

### Preparative Examples 1 and 2. Design of SARS-CoV-2 Universal Antigen Polypeptide Sequences

To design a universal antigen sequence universally applicable to various SARS-CoV-2 variants, a molecular phylogenetic analysis based on Bayesian inference was performed using approximately 2,000 target sequences including all genotypes of SARS-CoV-2, specifically a S1 subunit of a spike protein which serves as a major antigen of SARS-COV-2, and derived a conserved consensus sequence. Based on this, a universal antigen sequence was designed to respond not only to wild-type SARS-CoV-2 and existing variants, but also to potential future variants.

Specifically, approximately 800 viral gene sequences were extracted from genetic information registered on Global Initiative on Sharing All Influenza Data (GISAID), i.e., database which shares SARS-CoV-2 genetic information. Through molecular phylogenetic analysis, spike protein sequences of circulating SARS-CoV-2 variants were derived, and it was confirmed that the sequences match the sequences of variants, designated by the World Health Organization (WHO) as VOC. Antigenic mutations were identified in the circulating SARS-CoV-2 variants, and the spike protein was segmented into N-terminal domain (NTD), receptor-binding domain (RBD), and S2 subunit. Sequences from each circulating SARS-CoV-2 variant were incorporated into the design to ensure that the universal antigen could elicit vaccine efficacy across multiple SARS-CoV-2 variants. As a result, a universal antigen was produced according to the present disclosure.

### Preparative Example 1. Design of SARS-CoV-2 Universal Antigen Polypeptide Sequence (1)

Through the above process, a SARS-CoV-2 universal antigen polypeptide of SEQ ID NO: 4 was designed using S1 subunit sequences derived from all genotypes of SARS-CoV-2 variants. In a SARS-CoV-2 universal antigen polypeptide of SEQ ID NO: 4, specifically N-terminal domain was designed using mutations in Alpha variant B.1.1.7 sequences (ΔHV69-70 and ΔY144), and receptor-binding domain was designed using mutations in Delta variant B.1.617.2 sequences (L452R and T478K), and stabilizing mutations (R682A and ARAR683-685).

SARS-CoV-2 universal antigen polypeptide
(Consensus; Css)
(SEQ ID NO: 4)
MFVFLVLLPLVSSQCVNLTTRTQLPPAYTNSFTRGVYYPDKVFRS
SVLHSTQDLFLPFFSNVTWFHAISGTNGTKRFDNPVLPFNDGVYF
ASTEKSNIIRGWIFGTTLDSKTQSLLIVNNATNVVIKVCEFQFCN
DPFLGVYHKNNKSWMESEFRVYSSANNCTFEYVSQPFLMDLEGKQ
GNFKNLREFVFKNIDGYFKIYSKHTPINLVRDLPQGFSALEPLVD
LPIGINITRFQTLLALHRSYLTPGDSSSGWTAGAAAYYVGYLQPR
TFLLKYNENGTITDAVDCALDPLSETKCTLKSFTVEKGIYQTSNF
RVQPTESIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGENCYFPLQSYGFQ
PTNGVGYQPYRVVVLSFELLHAPATVCGPKKSTNLVKNKCVNFNF
NGLTGTGVLTESNKKFLPFQQFGRDIADTTDAVRDPQTLEILDIT
PCSFGGVSVITPGTNTSNQVAVLYQGVNCTEVPVAIHADQLTPTW
RVYSTGSNVFQTRAGCLIGAEHVNNSYECDIPIGAGICASYQTQT
NSPA

### Preparative Example 2. Design of SARS-CoV-2 Universal Antigen Polypeptide Sequence (2)

Through the above process, a SARS-CoV-2 universal antigen polypeptide of SEQ ID NO: 5 was designed using S1 subunit sequences derived from all genotypes of SARS-CoV-2 variants. In a SARS-CoV-2 universal antigen polypeptide of SEQ ID NO: 5, specifically N-terminal domain was designed using mutations in Omicron variant sequences (T19I, Δ24-26, ΔHV69-70, G142D), and receptor-binding domain and C-terminal domain were designed using mutations in Omicron variant BQ.1.1 and BN.1 sequences (G339H, R346T, S371F, S373P, S375F, T376A, D405N, R408S, K417N, N440K, G446S, N460K, S477N, T478K, E484A, Q498R, N501Y, Y505H, D614G, P681H), and stabilizing mutations (R682A and ΔRAR683-685).

SARS-CoV-2 universal antigen polypeptide
(Chimeric; Chi)
(SEQ ID NO: 5)
MFVFLVLLPLVSSQCVNLITRTQSYTNSFTRGVYYPDKVFRSSVL
HSTQDLFLPFFSNVTWFHAIHVSGTNGTKRFDNPVLPFNDGVYFA
STEKSNIIRGWIFGTTLDSKTQSLLIVNNATNVVIKVCEFQFCND
PFLDVYYHKNNKSWMESEFRVYSSANNCTFEYVSQPFLMDLEGKQ
GNFKNLREFVFKNIDGYFKIYSKHTPINLGRDLPQGFSALEPLVD
LPIGINITRFQTLLALHRSYLTPGDSSSGWTAGAAAYYVGYLQPR
TFLLKYNENGTITDAVDCALDPLSETKCTLKSFTVEKGIYQTSNF
RVQPTESIVRFPNITNLCPFHEVFNATTFASVYAWNRKRISNCVA
DYSVLYNFAPFFAFKCYGVSPTKLNDLCFTNVYADSFVIRGNEVS
QIAPGQTGNIADYNYKLPDDFTGCVIAWNSNKLDSKVSGNYNYLY
RLFRKSKLKPFERDISTEIYQAGNKPCNGVAGFNCYFPLQSYGFR
PTYGVGHQPYRVVVLSFELLHAPATVCGPKKSTNLVKNKCVNFNF
NGLTGTGVLTESNKKFLPFQQFGRDIADTTDAVRDPQTLEILDIT
PCSFGGVSVITPGTNTSNQVAVLYQGVNCTEVPVAIHADQLTPTW
RVYSTGSNVFQTRAGCLIGAEHVNNSYECDIPIGAGICASYQTQT
NSHA

### Preparative Example 3. Design of Optimized SARS-CoV-2 Universal Antigen Sequence-Derivation of Amino Acid Substitution Positions for Enhanced Structural Stability

Based on SARS-CoV-2 universal antigen polypeptide sequences of SEQ ID NOS: 4 and 5 derived in Preparative Examples 1 or 2, structural modeling of an antigen protein was performed using deep learning techniques. To enhance structural stability, an optimized SARS-CoV-2 universal antigen sequence was designed using in-silico methods.

Specifically, AlphaFold2, a deep learning-based in-silico analysis tool, was used to model tertiary structure of a S1 domain of a SARS-CoV-2 spike protein. Based on predicted tertiary structure and sequence, in-silico analysis was conducted using pSUFER and FuncLib servers to calculate sequence variation data and atom-based Rosetta structural energy changes of the protein. As a result, 1,000 candidate protein variants with mutations predicted to improve protein folding stability were derived. Among these, five top-ranked amino acid variants, one variant common across all 1,000 candidates, and one variant common among the five top-ranked variants (totaling seven amino acid variants) were selected as a primary candidate group. Physiochemical properties of the primary candidate group, which relate to protein expression level and RNA stability such as solubility, cavity formation, and RNA secondary structure were further predicted using in-silico tools using Aggrescan 3D (A3D), CASTp-3.0, and RNAfold servers. Based on these predictions, one variant was finally selected for each of the universal antigens of SEQ ID NOS: 4 and 5. As mutation sites introduced into the selected variant, five positions in a universal antigen of SEQ ID NO: 4, and nine positions in a universal antigen of SEQ ID NO: 5 were derived, and two sites were commonly found in sequences of SEQ ID NOS: 4 and 5.

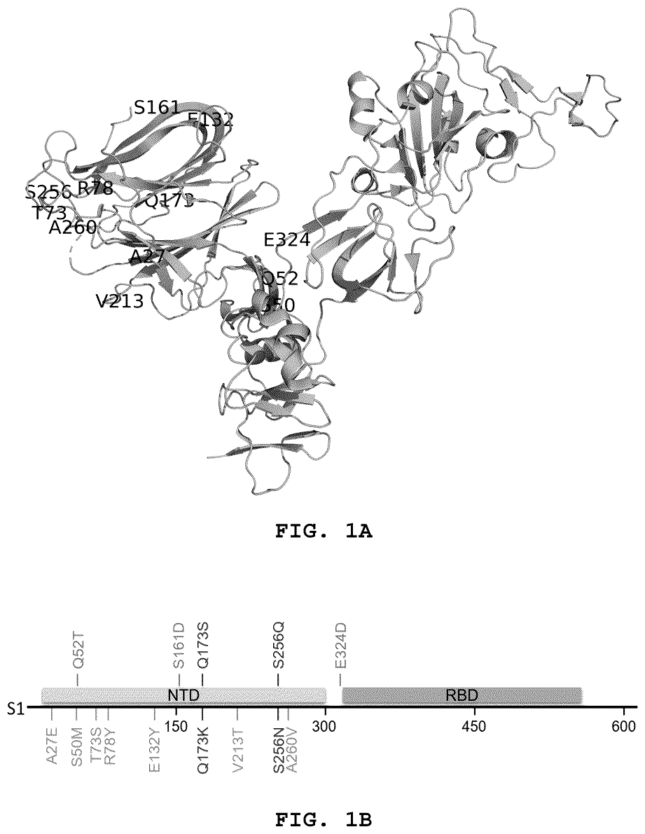

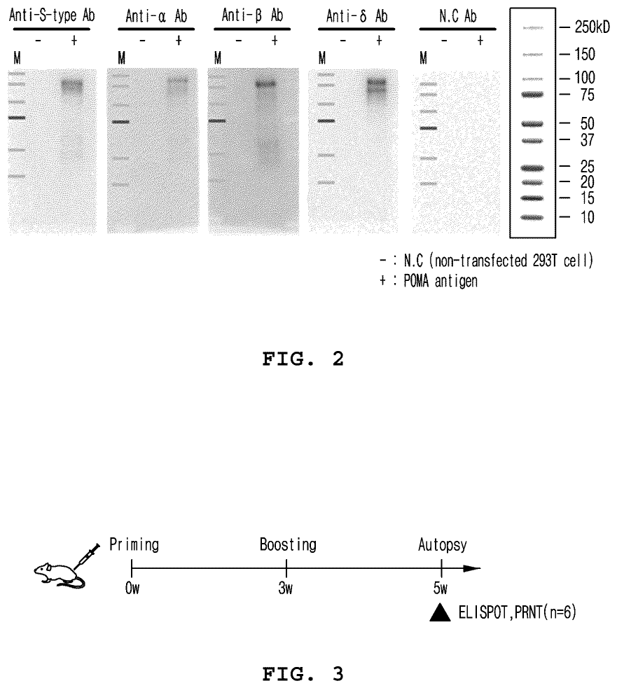

Specifically, the final five amino acid substitution positions selected for the universal antigen polypeptide of SEQ ID NO: 4 were as follows: Q52T substitution at position 52 (glutamine to threonine); S158D substitution at position 158 (serine to aspartic acid); Q170S substitution at position 170 (glutamine to serine); S253Q substitution at position 253 (serine to glutamine); and E321D substitution at position 321 (glutamic acid to aspartic acid), provided that the position numberings start from N-terminus of the amino acid sequences (see FIG. 1A-FIG. 1B).

In SEQ ID NO: 4, the structural stability-optimized sequence incorporating all five amino acid substitutions corresponds to SEQ ID NO: 2 below.

SARS-CoV-2 universal antigen polypeptide
(Consensus_dsg; Css_dsg)
(SEQ ID NO: 2)
MFVFLVLLPLVSSQCVNLTTRTQLPPAYTNSFTRGVYYPDKVFRS
SVLHSTTDLFLPFFSNVTWFHAISGTNGTKRFDNPVLPFNDGVYF
ASTEKSNIIRGWIFGTTLDSKTQSLLIVNNATNVVIKVCEFQFCN
DPFLGVYHKNNKSWMESEFRVYDSANNCTFEYVSSPFLMDLEGKQ
GNFKNLREFVFKNIDGYFKIYSKHTPINLVRDLPQGFSALEPLVD
LPIGINITRFQTLLALHRSYLTPGDSSQGWTAGAAAYYVGYLQPR
TFLLKYNENGTITDAVDCALDPLSETKCTLKSFTVEKGIYQTSNF
RVQPTDSIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGFNCYFPLQSYGFQ
PTNGVGYQPYRVVVLSFELLHAPATVCGPKKSTNLVKNKCVNFNF
NGLTGTGVLTESNKKFLPFQQFGRDIADTTDAVRDPQTLEILDIT
PCSFGGVSVITPGTNTSNQVAVLYQGVNCTEVPVAIHADQLTPTW
RVYSTGSNVFQTRAGCLIGAEHVNNSYECDIPIGAGICASYQTQT
NSPA

Furthermore, nine final amino acid substitution positions were derived in the universal antigen polypeptide of SEQ ID NO: 5. These substitutions were as follows: S24E substitution at position 24 (serine to glutamic acid); S47M substitution at position 47 (serine to methionine); T70S substitution at position 70 (threonine to serine); R75Y substitution at position 75 (arginine to tyrosine); E129Y substitution at position 129 (glutamic acid to tyrosine); Q170K substitution at position 170 (glutamine to lysine); G210T substitution at position 210 (glycine to threonine); S253N substitution at position 253 (serine to asparagine); and A257V substitution at position 257 (alanine to valine), provided that the position numberings start from N-terminus of the amino acid sequence.

In SEQ ID NO: 5, the structural stability-optimized sequence incorporating all nine amino acid substitutions corresponds to SEQ ID NO: 3 below.

SARS-CoV-2 universal antigen polypeptide
(Chimeric dsg; Chi_dsg)
(SEQ ID NO: 3)
MFVFLVLLPLVSSQCVNLITRTQEYTNSFTRGVYYPDKVFRSSVL
HMTQDLFLPFFSNVTWFHAIHVSGSNGTKYFDNPVLPFNDGVYFA
STEKSNIIRGWIFGTTLDSKTQSLLIVNNATNVVIKVCYFQFCND
PFLDVYYHKNNKSWMESEFRVYSSANNCTFEYVSKPFLMDLEGKQ
GNFKNLREFVFKNIDGYFKIYSKHTPINLTRDLPQGFSALEPLVD
LPIGINITRFQTLLALHRSYLTPGDSSNGWTVGAAAYYVGYLQPR
TFLLKYNENGTITDAVDCALDPLSETKCTLKSFTVEKGIYQTSNF
RVQPTESIVRFPNITNLCPFHEVFNATTFASVYAWNRKRISNCVA
DYSVLYNFAPFFAFKCYGVSPTKLNDLCFTNVYADSFVIRGNEVS
QIAPGQTGNIADYNYKLPDDFTGCVIAWNSNKLDSKVSGNYNYLY
RLFRKSKLKPFERDISTEIYQAGNKPCNGVAGFNCYFPLQSYGFR
PTYGVGHQPYRVVVLSFELLHAPATVCGPKKSTNLVKNKCVNFNF
NGLTGTGVLTESNKKFLPFQQFGRDIADTTDAVRDPQTLEILDIT
PCSFGGVSVITPGTNTSNQVAVLYQGVNCTEVPVAIHADQLTPTW
RVYSTGSNVFQTRAGCLIGAEHVNNSYECDIPIGAGICASYQTQT
NSHA

Based on the derived amino acid substitution positions, a sequence alignment was performed using SEQ ID NO: 1 as a reference sequence, in comparison with SEQ ID NOS: 2 and 3. As a result, the amino acid substitution positions were as follows: substitution to glutamic acid (E) at position 27; substitution to methionine (M) at position 50; substitution to threonine (T) at position 52; substitution to serine(S) at position 73; substitution to tyrosine (Y) at position 78; substitution to tyrosine (Y) at position 132; substitution to aspartic acid (D) at position 161; substitution to serine(S) or lysine (K) at position 173; substitution to threonine (T) at position 213; substitution to glutamine (Q) or asparagine (N) at position 256; substitution to valine (V) at position 260; and substitution to aspartic acid (D) at position 324), provided that the position numberings start from N-terminus of the amino acid (see Table 7).

TABLE 7
Results of Sequence Alignment and Amino Acid Substitution Positions

| | 27 | 50 | 52 | 73 | 78 | 132 | 161 | 173 | 213 | 256 | 260 | 324 |
|---|---|---|---|---|---|---|---|---|---|---|---|---|
| SEQ ID | E | M | T | S | Y | Y | D | S/K | T | Q/N | V | D |
| NO: 1 | | | | | | | | | | | | |
| SEQ ID | | | T | | | | D | S | | Q | | D |
| NO: 2 | | | | | | | | | | | | |
| SEQ ID | E | M | | S | Y | Y | | K | T | N | V | |
| NO: 3 | | | | | | | | | | | | |

The positions of amino acid substitutions that commonly occurred in both SEQ ID NOS: 2 and 3 are summarized in Table 8. In particular, positions 173 and 256, numbered relative to SEQ ID NO: 1, represent common substitution sites identified in both sequences, which were found to be highly likely to serve as universal stabilizing sites of the spike protein.

TABLE 8
Summary of Amino Acid Substitution Positions

| | | | | | | | | | | | | |
|---|---|---|---|---|---|---|---|---|---|---|---|---|
| SEQ ID | 27 | 50 | 52 | 73 | 78 | 132 | 161 | 173 | 213 | 256 | 260 | 324 |
| NO: 1 | | | | | | | | | | | | |
| SEQ ID | | | 52 | | | 158 | 170 | | 253 | | 321 | |
| NO: 2 | | | | | | | | | | | | |
| SEQ ID | 24 | 47 | | 70 | 75 | 129 | | 170 | 210 | 253 | 257 | |
| NO: 3 | | | | | | | | | | | | |

### Preparative Examples 4 and 5. Derivation of Full-Length S Antigen Protein Sequence Including SARS-CoV-2 Universal Antigen Polypeptide

For each of an optimized design sequences (SEQ ID NOS: 2 and 3) prepared in the Preparative Example 3 to enhance structural stability, a full-length S antigen protein sequence incorporating the respective optimized sequence was derived.

### Preparative Example 4. Derivation of Full-Length S Antigen Protein Sequence Including SARS-CoV-2 Universal Antigen Polypeptide of SEQ ID NO: 2

Using the SARS-CoV-2 universal antigen polypeptide of SEQ ID NO: 2, designed in the Preparative Example 3, as a S1 domain, a sequences of a full-length S antigen protein incorporating an S2 domain containing 6P structural stabilization mutations was derived. Specifically, a S2 subunit includes the D614G mutation, in which aspartic acid at position 614 of a wild-type SARS-CoV-2 spike protein is substituted with glycine. Additionally, the S2 subunit of selected universal spike proteins was engineered to include HexaPro mutations (F817P, A892P, A899P, A942P, K986P, V987P), as described by Hsieh et al. (Science 369:1501-05, 2020), along with a mutation at the S1/S2 cleavage site (682RRAR685 substituted with A), for enhancing structural stability.

The amino acid sequence of the full-length S antigen protein, comprising the SARS-CoV-2 universal antigen polypeptide of SEQ ID NO: 2 and prepared through the above process, corresponds to SEQ ID NO: 10 below.

Full-length S antigen protein sequence
including polypeptide of SEQ ID NO: 2
(Consensus_dsg; Css_dsg) (N = C)
(SEQ ID NO: 10)
MFVFLVLLPLVSSQCVNLTTRTQLPPAYTNSFTRGVYYPDKVFRS
SVLHSTTDLFLPFFSNVTWFHAISGTNGTKRFDNPVLPFNDGVYF
ASTEKSNIIRGWIFGTTLDSKTQSLLIVNNATNVVIKVCEFQFCN
DPFLGVYHKNNKSWMESEFRVYDSANNCTFEYVSSPFLMDLEGKQ
GNFKNLREFVFKNIDGYFKIYSKHTPINLVRDLPQGFSALEPLVD
LPIGINITRFQTLLALHRSYLTPGDSSQGWTAGAAAYYVGYLQPR
TFLLKYNENGTITDAVDCALDPLSETKCTLKSFTVEKGIYQTSNF
RVQPTDSIVRFPNITNLCPFGEVFNATRFASVYAWNRKRISNCVA
DYSVLYNSASFSTFKCYGVSPTKLNDLCFTNVYADSFVIRGDEVR
QIAPGQTGKIADYNYKLPDDFTGCVIAWNSNNLDSKVGGNYNYRY
RLFRKSNLKPFERDISTEIYQAGSKPCNGVEGENCYFPLQSYGFQ
PTNGVGYQPYRVVVLSFELLHAPATVCGPKKSTNLVKNKCVNFNF
NGLTGTGVLTESNKKFLPFQQFGRDIADTTDAVRDPQTLEILDIT
PCSFGGVSVITPGTNTSNQVAVLYQGVNCTEVPVAIHADQLTPTW
RVYSTGSNVFQTRAGCLIGAEHVNNSYECDIPIGAGICASYQTQT
NSPASVASQSIIAYTMSLGAENSVAYSNNSIAIPTNFTISVTTEI
LPVSMTKTSVDCTMYICGDSTECSNLLLQYGSFCTQLNRALTGIA
VEQDKNTQEVFAQVKQIYKTPPIKDFGGFNFSQILPDPSKPSKRS
PIEDLLFNKVTLADAGFIKQYGDCLGDIAARDLICAQKFNGLTVL
PPLLTDEMIAQYTSALLAGTITSGWTFGAGPALQIPFPMQMAYRF
NGIGVTQNVLYENQKLIANQFNSAIGKIQDSLSSTPSALGKLQDV
VNQNAQALNTLVKQLSSNFGAISSVLNDILSRLDPPEAEVQIDRL
ITGRLQSLQTYVTQQLIRAAEIRASANLAATKMSECVLGQSKRVD
FCGKGYHLMSFPQSAPHGVVFLHVTYVPAQEKNFTTAPAICHDGK
AHFPREGVFVSNGTHWFVTQRNFYEPQIITTDNTFVSGNCDVVIG
IVNNTVYDPLQPELDSFKEELDKYFKNHTSPDVDLGDISGINASV
VNIQKEIDRLNEVAKNINESLIDLQELGKYEQYIKWPWYIWLGFI
AGLIAIVMVTIMLCCMTSCCSCLKGCCSCGSCCKFDEDDSEPVLK
GVKLHYT

### Preparative Example 5. Derivation of Full-Length S Antigen Protein Sequence Including SARS-CoV-2 Universal Antigen Polypeptide of SEQ ID NO: 3

Using the SARS-CoV-2 universal antigen polypeptide of SEQ ID NO: 3, designed in the Preparative Example 3, as a S1 domain, a full-length S antigen protein sequence incorporating an S2 domain containing structural stabilization mutations was derived. Specifically, a S2 subunit includes the D614G mutation, in which aspartic acid at position 614 of a wild-type SARS-CoV-2 spike protein is substituted with glycine. Additionally, a S2 subunit of selected universal spike proteins was engineered to include HexaPro mutations (F817P, A892P, A899P, A942P, K986P, V987P), as described by Hsieh et al. (Science 369:1501-05, 2020), along with a mutation at the S1/S2 cleavage site (682RRAR685 substituted with A), for enhancing structural stability.

The amino acid sequence of the full-length S antigen protein, including the SARS-CoV-2 universal antigen polypeptide of SEQ ID NO: 3 and prepared through the above process, is shown below as SEQ ID NO: 11.

Full-length S antigen protein sequence
including polypeptide of SEQ ID NO: 3
(Chimeric_dsg; Chi_dsg) (N = C)
(SEQ ID NO: 11)
MFVFLVLLPLVSSQCVNLITRTQEYTNSFTRGVYYPDKVFRSSVL
HMTQDLFLPFFSNVTWFHAIHVSGSNGTKYFDNPVLPFNDGVYFA
STEKSNIIRGWIFGTTLDSKTQSLLIVNNATNVVIKVCYFQFCND
PFLDVYYHKNNKSWMESEFRVYSSANNCTFEYVSKPFLMDLEGKQ
GNFKNLREFVFKNIDGYFKIYSKHTPINLTRDLPQGFSALEPLVD
LPIGINITRFQTLLALHRSYLTPGDSSNGWTVGAAAYYVGYLQPR
TFLLKYNENGTITDAVDCALDPLSETKCTLKSFTVEKGIYQTSNF
RVQPTESIVRFPNITNLCPFHEVFNATTFASVYAWNRKRISNCVA
DYSVLYNFAPFFAFKCYGVSPTKLNDLCFTNVYADSFVIRGNEVS
QIAPGQTGNIADYNYKLPDDFTGCVIAWNSNKLDSKVSGNYNYLY
RLFRKSKLKPFERDISTEIYQAGNKPCNGVAGFNCYFPLQSYGFR
PTYGVGHQPYRVVVLSFELLHAPATVCGPKKSTNLVKNKCVNFNF
NGLTGTGVLTESNKKFLPFQQFGRDIADTTDAVRDPQTLEILDIT
PCSFGGVSVITPGTNTSNQVAVLYQGVNCTEVPVAIHADQLTPTW
RVYSTGSNVFQTRAGCLIGAEHVNNSYECDIPIGAGICASYQTQT
NSHASVASQSIIAYTMSLGAENSVAYSNNSIAIPTNFTISVTTEI
LPVSMTKTSVDCTMYICGDSTECSNLLLQYGSFCTQLNRALTGIA
VEQDKNTQEVFAQVKQIYKTPPIKDFGGFNFSQILPDPSKPSKRS
PIEDLLFNKVTLADAGFIKQYGDCLGDIAARDLICAQKFNGLTVL
PPLLTDEMIAQYTSALLAGTITSGWTFGAGPALQIPFPMQMAYRE
NGIGVTQNVLYENQKLIANQFNSAIGKIQDSLSSTPSALGKLQDV
VNQNAQALNTLVKQLSSNFGAISSVLNDILSRLDPPEAEVQIDRL
ITGRLQSLQTYVTQQLIRAAEIRASANLAATKMSECVLGQSKRVD
FCGKGYHLMSFPQSAPHGVVFLHVTYVPAQEKNFTTAPAICHDGK
AHFPREGVFVSNGTHWFVTQRNFYEPQIITTDNTFVSGNCDVVIG
IVNNTVYDPLQPELDSFKEELDKYFKNHTSPDVDLGDISGINASV
VNIQKEIDRLNEVAKNLNESLIDLQELGKYEQYIKWPWYIWLGFI
AGLIAIVMVTIMLCCMTSCCSCLKGCCSCGSCCKFDEDDSEPVLK
GVKLHYT

### Example 1. Confirmation of Universality of SARS-CoV-2 Universal Antigen Polypeptide

To confirm the universality of the SARS-CoV-2 universal antigen polypeptide of SEQ ID NO: 4, designed in the Preparative Example 1, the following experiment was conducted.

The SARS-CoV-2 universal antigen polypeptide of SEQ ID NO: 4 designated in the Preparative Example 1, was transfected into HEK293T cells to express a protein containing a universal antigen sequence. Subsequently, hamsters were intranasally infected with SARS-CoV-2 strains obtained from the National Culture Collection for Pathogens (NCCP), including an S type (Wuhan strain, NCCP No. 43326), Alpha variant (NCCP No. 43381), Beta variant (NCCP No. 43382), and Delta variant (NCCP No. 43390). Two weeks after infection, blood samples were collected to obtain antisera specific to each variant. Western blot analysis was performed using the collected antisera, and it was confirmed that all antisera from the S type (Wuhan strain), Alpha, Beta, and Delta variants of SARS-CoV-2 bound to the universal antigen. As a result, the universality of the antigen was also confirmed (see FIG. 2).

### Preparative Example 6. Production of SARS-CoV-2 Universal mRNA Vaccine

### Preparative Example 6-1. Preparation of SARS-CoV-2 Universal mRNA Vaccine

Using the SARS-CoV-2 universal antigen polypeptide of SEQ ID NO: 2, designed in the Preparative Example 3, and the full-length S antigen protein sequence of SEQ ID NO: 10, produced in the Preparative Example 4, an mRNA vaccine was produced by cloning into an IVT vector for mRNA vaccine production.

Specifically, the SARS-CoV-2 universal antigen sequence of SEQ ID NO: 2 was codon-optimized to synthesize corresponding gene (DNA). The synthesized gene was inserted into the IVT vector for mRNA vaccine production using an overlapping cloning method. PCR amplification was performed to linearize an antigen sequence within the IVT vector, and to add a poly(A) tail. The resulting linear DNA was transcribed into mRNA of SEQ ID NO: 12 using an in vitro transcription kit using T7 RNA polymerase. The synthesized mRNA was purified using an RNeasy Mini Kit (74104, Qiagen), and gene size and RNA quality were confirmed. After that, the synthesized mRNA was stored at −20° C.

mRNA Corresponding to SEQ ID NO: 10
(Consensus dsg; Css dsg) (5′ → 3′)
(SEQ ID NO: 12)
AUGUUCGUGUUUCUGGUCCUGCUGCCUCUGGUGUCUAGCCAGUGC
GUGAACCUGACCACCCGGACCCAACUGCCUCCUGCCUACACCAAU
AGCUUUACAAGAGGCGUGUAUUACCCCGAUAAGGUGUUCAGAAGC
AGCGUGCUGCACAGCACAACCGACCUGUUCCUGCCUUUCUUCAGC
AACGUGACCUGGUUCCACGCCAUCAGCGGAACUAACGGCACCAAG
AGAUUCGACAACCCCGUGCUGCCAUUCAACGACGGCGUUUAUUUC
GCCUCUACCGAGAAGAGCAACAUCAUCAGAGGCUGGAUCUUCGGC
ACCACCCUGGACAGCAAAACCCAGAGUCUGCUGAUCGUGAAUAAC
GCCACCAACGUCGUCAUCAAGGUGUGCGAGUUCCAGUUCUGCAAC
GACCCUUUCUUGGGCGUGUACCACAAGAACAACAAGUCCUGGAUG
GAAAGCGAAUUUAGAGUGUACGACUCUGCCAACAACUGCACCUUC
GAGUACGUGUCCAGCCCUUUCCUGAUGGACCUUGAAGGAAAACAG
GGCAACUUCAAGAAUCUGAGGGAAUUCGUGUUCAAAAACAUCGAC
GGCUACUUCAAGAUCUACAGCAAGCACACCCCUAUCAACCUGGUG
CGGGACUUGCCGCAAGGCUUCAGCGCCCUGGAACCUCUGGUGGAC
CUGCCCAUCGGCAUUAACAUCACCCGGUUCCAGACCCUGCUGGCC
CUCCACAGAUCCUACCUGACCCCUGGCGAUAGCUCUCAGGGCUGG
ACCGCCGGCGCCGCUGCUUACUACGUGGGAUACCUGCAGCCUAGA
ACAUUCCUGCUGAAGUACAACGAGAACGGCACAAUCACCGACGCC
GUGGACUGCGCCCUGGACCCCCUGUCUGAGACAAAGUGCACACUG
AAAAGCUUCACCGUGGAAAAAGGCAUCUACCAAACCAGCAAUUUC
AGAGUGCAGCCUACAGAUUCCAUCGUGCGGUUUCCAAAUAUCACA
AACCUGUGUCCUUUUGGCGAGGUGUUCAACGCCACCAGAUUCGCC
UCCGUGUAUGCCUGGAACAGAAAGAGAAUCAGCAACUGCGUGGCC
GACUACAGCGUGCUGUACAACAGCGCCUCCUUUAGCACAUUCAAG
UGCUACGGCGUCAGCCCUACAAAGCUGAACGACCUGUGCUUCACC
AACGUCUACGCCGACAGCUUUGUGAUCCGGGGCGACGAGGUGCGG
CAGAUCGCCCCUGGCCAGACUGGCAAGAUCGCUGACUACAAUUAU
AAGCUGCCAGAUGACUUCACCGGAUGUGUGAUCGCCUGGAACUCC
AACAACCUGGAUAGCAAGGUGGGCGGAAAUUACAACUACAGAUAC
CGGCUGUUCCGGAAGUCUAACCUGAAGCCUUUUGAAAGAGAUAUU
AGCACUGAGAUCUACCAGGCCGGCAGCAAGCCCUGUAAUGGAGUG
GAAGGCUUCAACUGCUACUUUCCUCUGCAAAGCUACGGGUUUCAG
CCUACAAAUGGCGUGGGCUACCAGCCCUACAGAGUGGUGGUGCUG
AGCUUCGAGCUGCUGCAUGCUCCUGCUACAGUGUGCGGCCCUAAG
AAGUCCACAAAUCUGGUGAAGAACAAAUGUGUGAACUUCAAUUUC
AACGGCCUGACAGGCACCGGCGUUCUGACAGAGAGCAACAAGAAA
UUCCUGCCCUUCCAGCAGUUCGGCAGAGACAUCGCAGAUACAACC
GAUGCCGUGAGAGAUCCCCAGACCCUGGAGAUCCUGGACAUCACC
CCCUGUAGCUUCGGCGGCGUUAGCGUGAUCACCCCAGGCACCAAU
ACCUCUAACCAGGUCGCUGUGCUGUACCAGGGAGUGAACUGCACC
GAGGUGCCUGUGGCUAUCCACGCCGAUCAGCUGACCCCCACAUGG
GGGUGUACUCUACAGGAAGCAAUGUGUUUCAGACCCGCGCCGGUU
GUCUGAUCGGCGCCGAGCACGUGAACAAUUCAUACGAGUGCGACA
UCCCUAUCGGAGCUGGCAUCUGCGCCUCCUACCAGACCCAGACCA
ACAGCCCCGCCUCUGUAGCCUCUCAAAGCAUCAUUGCCUACACAA
UGAGCCUGGGCGCCGAGAACUCCGUUGCUUAUAGUAAUAAUAGCA
UUGCAAUCCCUACCAACUUCACCAUCAGCGUGACCACAGAGAUUC
UGCCUGUGAGCAUGACCAAGACCUCCGUGGACUGUACCAUGUACA
UCUGUGGCGACUCGACUGAAUGCAGCAACCUGCUGCUCCAAUACG
GCAGCUUCUGCACACAGCUGAACCGGGCUCUGACCGGCAUCGCCG
UGGAACAGGACAAGAACACCCAAGAGGUGUUUGCCCAGGUGAAGC
AGAUUUACAAGACCCCUCCCAUCAAGGACUUCGGCGGUUUCAACU
UUAGCCAGAUCCUGCCUGAUCCUUCUAAACCCAGCAAGCGGAGCC
CCAUCGAGGACCUGCUGUUCAACAAGGUGACCCUGGCUGACGCCG
GAUUCAUCAAGCAGUAUGGCGACUGUCUGGGCGAUAUCGCUGCCA
GAGAUCUCAUCUGUGCCCAGAAGUUCAACGGACUGACUGUGCUAC
CACCCUUACUGACCGAUGAGAUGAUCGCUCAGUACACAUCUGCCC
UGCUGGCUGGGACCAUCACCAGCGGCUGGACAUUCGGCGCCGGCC
CCGCCCUGCAAAUCCCGUUUCCUAUGCAGAUGGCCUAUAGAUUCA
ACGGAAUCGGCGUGACCCAGAACGUCCUGUAUGAGAACCAGAAGC
UGAUCGCUAAUCAGUUCAACAGCGCAAUCGGCAAAAUCCAAGACU
CUCUGAGCAGCACACCCUCCGCUCUGGGCAAGCUGCAGGAUGUGG
UGAACCAAAACGCCCAAGCCCUUAACACCCUGGUGAAACAGCUGA
GCAGCAACUUUGGUGCCAUCAGCAGCGUGCUUAACGACAUUCUGU
CUAGGCUGGACCCCCCUGAGGCCGAAGUGCAGAUCGACAGACUGA
UCACCGGAAGACUGCAGAGCCUGCAAACCUACGUGACCCAACAGU
UGAUCAGAGCCGCUGAGAUCCGGGCGAGCGCCAACCUGGCCGCCA
CAAAGAUGAGCGAAUGUGUGCUGGGGCAGAGCAAAAGAGUGGACU
UCUGCGGCAAAGGCUAUCACCUGAUGAGCUUUCCUCAGAGCGCCC
CCCACGGCGUGGUGUUCCUGCACGUGACAUACGUGCCUGCUCAGG
AGAAGAACUUCACCACCGCCCCUGCCAUCUGCCACGACGGCAAGG
CUCACUUCCCAAGAGAAGGCGUGUUCGUCAGCAAUGGCACCCACU
GGUUCGUGACCCAAAGAAACUUCUACGAGCCUCAGAUCAUCACAA
CCGACAACACAUUCGUGAGCGGGAACUGCGACGUGGUAAUCGGCA
UUGUGAACAAUACCGUGUACGACCCUCUGCAGCCAGAACUGGAUU
CCUUCAAGGAAGAGCUGGACAAGUACUUUAAGAACCACACAAGCC
CUGACGUGGACCUGGGCGACAUAAGCGGUAUCAACGCCUCAGUGG
UCAACAUCCAGAAAGAGAUCGACAGGUUAAACGAAGUGGCCAAGA
ACCUGAAUGAGUCCCUGAUCGAUCUGCAGGAGCUGGGCAAGUACG
AACAGUACAUCAAGUGGCCUUGGUACAUCUGGCUGGGAUUCAUUG
CCGGAUUGAUCGCCAUCGUCAUGGUGACCAUCAUGCUGUGCUGCA
UGACGAGCUGCUGCUCUUGCCUGAAGGGCUGCUGUAGCUGUGGCU
CCUGUUGUAAAUUCGACGAAGAUGACAGCGAGCCCGUGCUGAAGG
GAGUGAAGCUGCAUUACACC

Furthermore, using the SARS-CoV-2 universal antigen polypeptide of SEQ ID NO: 3 designed in Preparative Example 3, and the full-length S antigen protein sequence of SEQ ID NO: 11 produced in Preparative Example 5, the same procedure as described above was performed to synthesize mRNA of SEQ ID NO: 13 as shown below.

mRNA corresponding to SEQ ID NO: 11
(Chimeric_dsg; Chi_dsg) (5′ → 3′)
(SEQ ID NO: 13)
AUGUUCGUGUUCCUGGUGCUGCUGCCUCUGGUGAGCAGCCAGUGC
GUGAAUCUGAUUACAAGAACUCAGGAGUACACUAACUCUUUCACC
AGAGGAGUGUACUACCCCGAUAAGGUGUUUAGGUCAAGCGUGCUG
CACAUGACUCAGGACCUGUUCCUGCCUUUCUUUUCUAAUGUGACC
UGGUUCCACGCAAUCCACGUGAGCGGCAGCAACGGCACGAAAUAU
UUCGAUAAUCCUGUGCUGCCCUUCAACGACGGCGUGUACUUCGCC
UCCACAGAGAAGUCAAACAUUAUUCGGGGGUGGAUCUUCGGCACC
ACACUGGACAGUAAAACCCAGUCCCUGCUGAUCGUGAACAAUGCU
ACCAACGUGGUGAUUAAGGUGUGCUACUUUCAGUUCUGCAAUGAU
CCAUUCCUGGAUGUGUACUACCACAAGAACAAUAAGUCCUGGAUG
GAAAGCGAAUUCAGAGUGUAUUCCUCUGCAAAUAACUGUACAUUC
GAGUACGUGUCCAAGCCAUUCCUCAUGGACCUUGAAGGCAAGCAG
GGGAAUUUUAAAAACCUGCGGGAGUUUGUGUUUAAGAAUAUCGAU
GGCUACUUCAAGAUUUAUUCCAAACACACUCCCAUCAACCUGACC
AGGGACCUGCCCCAGGGCUUUUCCGCCCUGGAGCCCCUGGUGGAC
CUGCCCAUUGGCAUUAACAUUACAAGAUUCCAGACCCUGCUGGCU
CUGCACAGGUCCUAUCUGACACCUGGCGAUAGCAGCAAUGGCUGG
ACCGUCGGGGCAGCCGCCUAUUACGUGGGAUACCUGCAGCCCCGG
ACCUUUCUGCUGAAGUACAAUGAGAACGGCACAAUCACAGACGCU
GUGGACUGCGCCCUGGACCCACUGAGCGAGACUAAGUGCACUCUG
AAAUCAUUCACUGUGGAGAAGGGGAUUUAUCAGACAUCUAACUUC
AGAGUCCAGCCUACCGAGAGUAUCGUGAGGUUCCCCAACAUCACC
AACCUGUGCCCUUUUCACGAGGUGUUUAACGCCACCACCUUCGCC
UCAGUGUAUGCCUGGAACAGGAAGAGAAUCAGCAAUUGCGUGGCA
GACUACUCCGUGCUGUACAACUUCGCACCCUUCUUCGCCUUUAAG
UGUUACGGCGUGAGCCCCACCAAACUGAACGAUCUGUGCUUCACA
AAUGUGUACGCCGAUAGUUUUGUGAUCCGGGGGAAUGAGGUGUCU
CAAAUCGCCCCCGGCCAGACCGGAAACAUCGCUGAUUAUAACUAC
AAGCUGCCCGAUGAUUUUACCGGGUGCGUGAUCGCCUGGAAUAGU
AAUAAGCUGGAUAGCAAAGUGUCUGGCAACUAUAACUAUCUGUAC
CGGCUGUUCAGGAAAUCCAAGCUGAAGCCCUUCGAGCGCGACAUC
UCUACCGAAAUUUACCAGGCCGGUAACAAGCCAUGUAACGGCGUG
GCUGGCUUUAAUUGCUACUUCCCACUGCAGAGCUACGGGUUCCGG
CCCACCUAUGGGGUGGGGCAUCAGCCCUACCGCGUGGUGGUGCUG
UCCUUCGAGCUGCUGCACGCCCCAGCUACCGUGUGCGGGCCCAAG
AAGUCAACUAAUCUGGUCAAGAACAAGUGUGUCAACUUCAACUUC
AAUGGCCUGACUGGGACAGGCGUGCUCACCGAGAGCAACAAGAAA
UUCCUGCCUUUUCAGCAGUUCGGCAGGGACAUCGCCGACACCACA
GAUGCCGUUCGGGACCCACAGACCCUGGAAAUCCUGGAUAUUACU
CCAUGCAGCUUCGGCGGCGUGUCCGUGAUUACACCUGGAACCAAC
ACCAGCAACCAGGUGGCCGUGCUGUACCAGGGCGUGAACUGCACC
GAGGUGCCUGUGGCAAUACACGCCGACCAGCUGACUCCCACAUGG
CGAGUGUAUUCAACCGGCUCCAAUGUGUUUCAGACUAGAGCCGGC
UGCCUGAUCGGAGCUGAGCACGUGAAUAACUCUUAUGAGUGCGAU
AUCCCCAUCGGAGCCGGCAUCUGCGCAUCCUAUCAGACACAGACA
AACAGCCACGCCAGCGUGGCCUCUCAAAGCAUCAUUGCCUACACA
AUGAGCCUGGGCGCCGAGAACUCCGUUGCUUAUAGUAAUAAUAGC
AUUGCAAUCCCUACCAACUUCACCAUCAGCGUGACCACAGAGAUU
CUGCCUGUGAGCAUGACCAAGACCUCCGUGGACUGUACCAUGUAC
AUCUGUGGCGACUCGACUGAAUGCAGCAACCUGCUGCUCCAAUAC
GGCAGCUUCUGCACACAGCUGAACCGGGCUCUGACCGGCAUCGCC
GUGGAACAGGACAAGAACACCCAAGAGGUGUUUGCCCAGGUGAAG
CAGAUUUACAAGACCCCUCCCAUCAAGGACUUCGGCGGUUUCAAC
UUUAGCCAGAUCCUGCCUGAUCCUUCUAAACCCAGCAAGCGGAGC
CCCAUCGAGGACCUGCUGUUCAACAAGGUGACCCUGGCUGACGCC
GGAUUCAUCAAGCAGUAUGGCGACUGUCUGGGCGAUAUCGCUGCC
AGAGACCUCAUCUGUGCCCAGAAGUUCAACGGACUGACUGUGCUA
CCACCCUUACUGACCGAUGAGAUGAUCGCUCAGUACACAUCUGCC
CUGCUGGCUGGGACCAUCACCAGCGGCUGGACAUUCGGCGCCGGC
CCCGCCCUGCAAAUCCCGUUUCCUAUGCAGAUGGCCUAUAGAUUC
AACGGAAUCGGCGUGACCCAGAACGUCCUGUAUGAGAACCAGAAG
CUGAUCGCUAAUCAGUUCAACAGCGCAAUCGGCAAAAUCCAAGAC
UCUCUGAGCAGCACACCCUCCGCUCUGGGCAAGCUGCAGGAUGUG
GUGAACCAAAACGCCCAAGCCCUUAACACCCUGGUGAAACAGCUG
AGCAGCAACUUUGGUGCCAUCAGCAGCGUGCUUAACGACAUUCUG
UCUAGGCUGGACCCCCCUGAGGCCGAAGUGCAGAUCGACAGACUG
AUCACCGGAAGACUGCAGAGCCUGCAAACCUACGUGACCCAACAG
UUGAUCAGAGCCGCUGAGAUCCGGGCGAGCGCCAACCUGGCCGCC
ACAAAGAUGAGCGAAUGUGUGCUGGGGCAGAGCAAAAGAGUGGAC
UUCUGCGGCAAAGGCUAUCACCUGAUGAGCUUUCCUCAGAGCGCC
CCCCACGGCGUGGUGUUCCUGCACGUGACAUACGUGCCUGCUCAG
GAGAAGAACUUCACCACCGCCCCUGCCAUCUGCCACGACGGCAAG
GCUCACUUCCCAAGAGAAGGCGUGUUCGUCAGCAAUGGCACCCAC
UGGUUCGUGACCCAAAGAAACUUCUACGAGCCUCAGAUCAUCACA
ACCGACAACACAUUCGUGAGCGGGAACUGCGACGUGGUAAUCGGC
AUUGUGAACAAUACCGUGUACGACCCUCUGCAGCCAGAACUGGAU
UCCUUCAAGGAAGAGCUGGACAAGUACUUUAAGAACCACACAAGC
CCUGACGUGGACCUGGGCGACAUAAGCGGUAUCAACGCCUCAGUG
GUCAACAUCCAGAAAGAGAUCGACAGGUUAAACGAAGUGGCCAAG
AACCUGAAUGAGUCCCUGAUCGAUCUGCAGGAGCUGGGCAAGUAC
GAACAGUACAUCAAGUGGCCUUGGUACAUCUGGCUGGGAUUCAUU
GCCGGAUUGAUCGCCAUCGUCAUGGUGACCAUCAUGCUGUGCUGC
AUGACGAGCUGCUGCUCUUGCCUGAAGGGCUGCUGUAGCUGUGGC
UCCUGUUGUAAAUUCGACGAAGAUGACAGCGAGCCCGUGCUGAAG
GGAGUGAAGCUGCAUUACACC

### Preparative Example 6-2. LNP Encapsulation of SARS-CoV-2 Universal mRNA Vaccine

LNP encapsulation was performed using a NanoAssemblr Benchtop Instrument (Precision Nanosystems Inc). Each ionizable lipid was prepared by dilution in ethanol, and mRNA was prepared by dilution in 10 mM citrate buffer. A mixture containing SM-102:DSPC:cholesterol:DMG-PEG2000 in a ratio of 50:10:38.5:1.5, was mixed with mRNA at a volume ratio of 1:3 for encapsulation. The resulting mRNA-LNP was diluted with 1×PBS, and concentrated by centrifugation using an Amicon Ultra-15 filter. To confirm LNP quality, particle size distribution of LNP particles was measured using a Nano-ZS zetasizer, and RNA concentration, and LNP encapsulation efficiency were determined using a Ribogreen assay (R11490, Invitrogen).

### Example 2. Evaluation of Immunogenicity of SARS-CoV-2 Universal mRNA Vaccine of SEQ ID NO: 12

### Example 2-1. Administration of SARS-Cov-2 Universal mRNA Vaccine

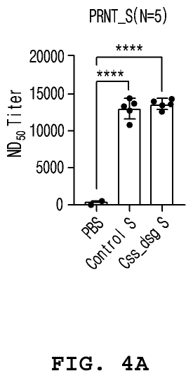

Immunogenicity of the SARS-CoV-2 universal mRNA vaccine of SEQ ID NO: 12, prepared in the Preparative Example 6, was evaluated using a mouse model. Specifically, female Balb/c mice (4 weeks old) were intramuscularly (I.M.) immunized with 5 μg of the SARS-CoV-2 universal mRNA vaccine twice at a 3-week interval. Two weeks after final immunization, serum and splenocytes were collected to assess humoral and cellular immunity (see FIG. 3). Mice were immunized using a wild-type SARS-CoV-2 S antigen sequence (Wuhan strains; control group) from Moderna in the same manner.

### Example 2-2. Confirmation of Humoral Immunity (Neutralizing Antibody Titer)

To evaluate whether a SARS-CoV-2 universal mRNA vaccine of SEQ ID NO: 12, prepared in the Preparative Example 6, induces a humoral immune response, neutralizing antibody titers were measured. Specifically, sera from immunized mice were inactivated at 56° C., and serially diluted two-fold in DMEM medium containing 2% FBS and 1% Penicillin-Streptomycin in a 96-well plate. Each of the following viruses such as a wild-type SARS-CoV-2 S strain (Wuhan), Delta variant (India), and Omicron variant (BA.5), was diluted to 400 PFU/ml, and mixed with the diluted sera at a 1:1 ratio, followed by incubation at 37° C. with 5% CO₂ for 1 hour to perform the neutralization step. One day prior to the assay, Vero-E6 cells were seeded at 1.8×10⁵ cells/well in a 12-well plate. The neutralized serum-virus mixtures were added to the plate, and incubated for 1 hour at 37° C. with 5% CO₂. After removing the mixtures, overlay medium, i.e., a MEM supplemented with 2% FBS, 1% Penicillin-Streptomycin, and 0.6% agarose was added, and incubated for 2 days. The overlay medium was then removed, and crystal violet staining solution was added, and left for 2 hours. After removing the staining solution, and drying the plate, plaques were counted to calculate neutralizing antibody titers.

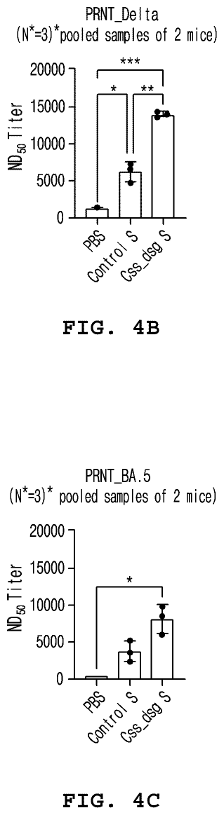

As shown in FIG. 4, the SARS-CoV-2 universal mRNA vaccine of the present invention, based on the early SARS-CoV-2 strain (denoted as “Consensus_dsg S” or “Css_dsg S” in FIG. 4), exhibited higher neutralizing antibody titers not only against a wild-type SARS-CoV-2 S strain (Wuhan), but also against Delta variant (India) and Omicron variant (BA.5), when compared to a conventional wild-type SARS-CoV-2 S strain vaccine (denoted as “Control S” in FIG. 4).

### Example 2-3. Confirmation of Cellular Immunity (IFN-γ Induction Capability)

To evaluate whether the SARS-CoV-2 universal mRNA vaccine of SEQ ID NO: 12, prepared in Preparative Example 6, induces a cellular immune response, an ELISpot assay was performed to measure virus-specific IFN-γ-secreting T cells from spleens of immunized mice. Specifically, spleens were harvested from mice immunized with a SARS-CoV-2 universal mRNA vaccine, and dissociated into single cells using a gentleMACS instrument in RPMI medium containing 10% FBS and 1% Penicillin-Streptomycin. The splenocytes were filtered through a 70 μm strainer, and centrifuged at 2,000 rpm for 5 minutes at 4° C. After removing supernatant, remained pellets were resuspended in 5 ml of ACK lysing buffer, vortexed, and incubated at 37° C. for 5 minutes. An additional 10 ml of RPMI medium containing 10% FBS and 1% Penicillin-Streptomycin was added, followed by centrifugation under the same conditions. Supernatant was discarded, and remained pellets were resuspended in 1-3 ml of RPMI medium containing 10% FBS and 1% Penicillin-Streptomycin. Splenocyte counts were determined using a LUNA cell counter. Splenocytes were seeded at 2.5×10⁵ cells/well in a 96-well plate in a Mouse IFN-γ ELISpot Plus Kit (R&D Systems). SARS-CoV-2 peptides were diluted to a final concentration of 1 μg/well, and added to the wells as stimulants. Mixtures of the cells and stimulants were incubated at 37° C. with 5% CO₂ for 12-48 hours. After incubation, the mixtures were removed, and the wells were washed with PBS. After that, primary antibodies diluted in PBS containing 0.5% FBS were diluted and added, followed by incubating at ambient temperature for 2 hours. After washing, ALP-conjugated secondary antibodies were diluted in PBS with 0.5% FBS and added, followed by incubating for 1 hour at ambient temperature. Following another wash, a sterile-filtered substrate solution (BCIP/NBT-plus) was added, and allowed to react for 10-30 minutes until spots appeared. The reaction was stopped by washing with distilled water, and the plates were thoroughly dried. The resulting spots were counted using a CTL automated reader.

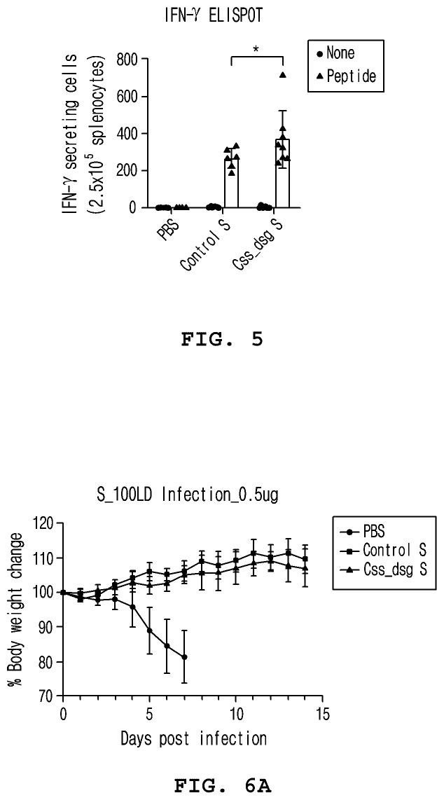

As a result of this experiment, the SARS-CoV-2 universal mRNA vaccine based on an early SARS-CoV-2 strain (denoted as “Consensus_dsg S” or “Css_dsg S” in FIG. 5) demonstrated a comparable level of good cellular immune response to that of the conventional wild-type SARS-CoV-2 S strain (Wuhan) vaccine (denoted as “Control S” in FIG. 5).

### Example 3. Evaluation of Protective Efficacy of SARS-CoV-2 Universal mRNA Vaccine of SEQ ID NO: 12

To evaluate the protective efficacy of the SARS-CoV-2 universal mRNA vaccine of SEQ ID NO: 12, prepared in the Preparative Example 6, female human ACE2 transgenic mice [B6.Cg-Tg (K18-ACE2) 2Prlmn/J], 5 weeks of age and susceptible to SARS-CoV-2 infection, were intramuscularly (I.M.) immunized twice with 0.5 μg of the vaccine candidate at a 3-week interval. Two weeks after the final immunization, the mice were challenged with 100 LD₅₀ doses of either a wild-type SARS-CoV-2 S strain (Wuhan) or Delta variant (India). After that, body weight and survival rate were monitored daily for 2 weeks.

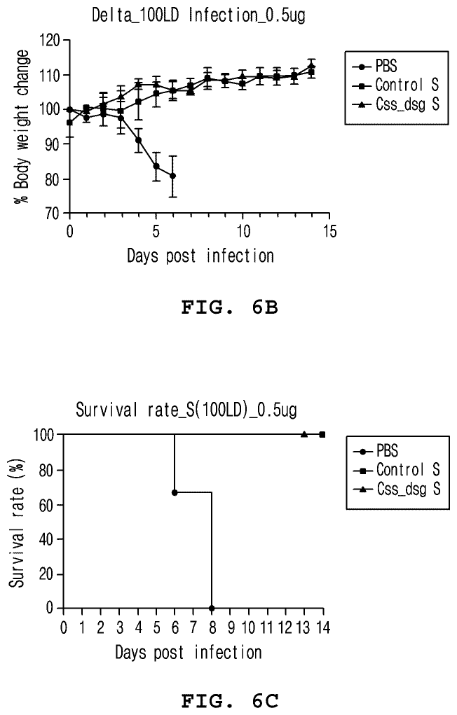

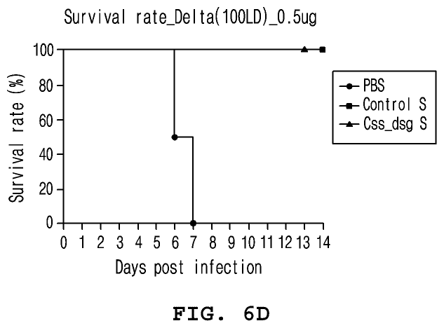

According to the results of this experiment, a PBS control group (non-immunized) exhibited rapid weight loss beginning 3-4 days after viral challenge, and all mice died within 9 days (see FIG. 6). In contrast, the group immunized with the SARS-CoV-2 universal mRNA vaccine based on an early strain of the present invention (denoted as “Consensus_dsg S” or “Css_dsg S” in FIG. 6) showed 100% survival rate without any weight loss following challenge with both a wild-type SARS-CoV-2 S strain (Wuhan) and Delta variant (India) (see FIG. 6).

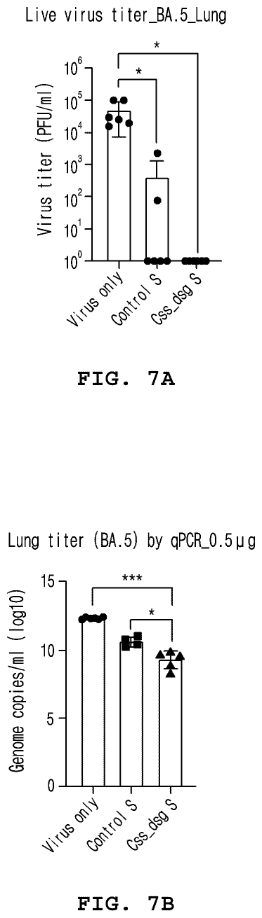

As shown in FIG. 7, mice were challenged with 3.6×10⁴ PFU of Omicron variant, and viral titers in lung tissues were measured 3 days post-challenge. High viral titers were measured in the PBS control group (non-immunized), and viral titers were also measured in a group immunized with a conventional wild-type SARS-CoV-2 S strain (Wuhan) vaccine (denoted as “Control S” in FIG. 4). In contrast, a group immunized with the SARS-CoV-2 universal mRNA vaccine based on the early strain of the present invention (denoted as “Consensus_dsg S” or “Css_dsg S” in FIG. 7) showed complete viral clearance in lung tissues. Furthermore, RT-qPCR analysis revealed a significantly reduced viral load in lung tissues of the SARS-CoV-2 universal vaccine compared to the group immunized with a conventional wild-type SARS-CoV-2 S strain (Wuhan) vaccine group (FIG. 7A˜FIG. 7B).

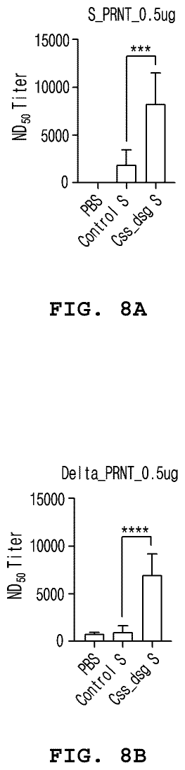

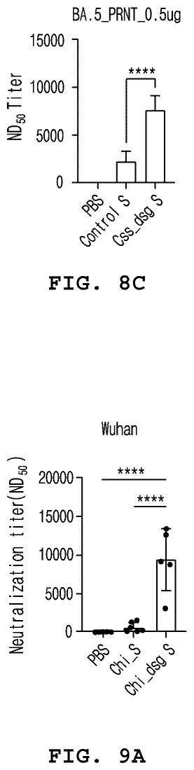

As shown in FIG. 8, neutralizing antibody titers were measured in sera collected prior to viral challenge, following two intramuscular (I.M.) immunizations with candidate vaccines. The SARS-CoV-2 universal mRNA vaccine based on the early strain of the present invention (denoted as “Consensus_dsg S” or “Css_dsg S” in FIG. 8) induced significantly higher neutralizing antibody titers not only against the wild-type SARS-CoV-2 S strain (Wuhan), but also against the Delta variant (India) and the Omicron variant (BA. 5), compared to the conventional wild-type SARS-CoV-2 S strain (Wuhan) vaccine (denoted as “Control S” in FIG. 8A˜FIG. 8C).

### Example 4. Evaluation of Immunogenicity of SARS-CoV-2 Universal mRNA Vaccine of SEQ ID NO: 13

To evaluate whether the SARS-CoV-2 universal mRNA vaccine of SEQ ID NO: 13, prepared in the Preparative Example 6, induces a humoral immune response, neutralizing antibody titers were measured. Specifically, sera from immunized mice were inactivated at 56° C., and serially diluted two-fold in DMEM medium containing 2% FBS and 1% Penicillin-Streptomycin in a 96-well plate. Each of the following viruses, i.e., SARS-CoV-2 S strain (Wuhan), Omicron variant (BA.5), and Omicron variant (BN.1) was diluted to 400 PFU/ml, and mixed with the diluted sera at a 1:1 ratio, followed by incubation at 37° C. with 5% CO₂ for 1 hour to perform a neutralization step. One day prior to this assay, Vero-E6 cells were seeded at 1.8×10⁵ cells/well in a 12-well plate. The neutralized serum-virus mixtures were added to the seeded cells, and incubated for 1 hour at 37° C. with 5% CO₂. After removing the serum-virus mixtures, overlay medium, i.e., MEM supplemented with 2% FBS, 1% Penicillin-Streptomycin, and 0.6% agarose was added, and incubated for 2 days. The overlay medium was then removed, and crystal violet staining solution was added, and left for 2 hours. After removing the staining solution, and drying the wells, plaques were counted to calculate the neutralizing antibody titers.

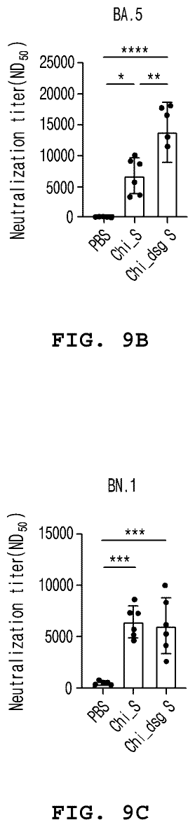

As a result of this experiment, the SARS-CoV-2 Omicron universal mRNA vaccine of the present invention (denoted as “Chimeric_dsg S” or “Chi_dsg S” in FIG. 9) exhibited higher neutralizing antibody titers not only against the SARS-CoV-2 S strain (Wuhan), but also against the Omicron variant (BA. 5) and the Omicron variant (BN. 1), compared to the Omicron Chimeric Spike vaccine (denoted as “Chimeric S” or “Chi S” in FIG. 9) (FIG. 9A˜ FIG. 9C).

### Example 5. Evaluation of Protective Efficacy of the SARS-CoV-2 Universal mRNA Vaccine of SEQ ID NO: 13

To evaluate whether the SARS-CoV-2 Omicron universal mRNA vaccine of SEQ ID NO: 13, prepared in the Preparative Example 6, induces a cellular immune response, an ELISpot assay was performed to measure virus-specific IFN-γ-secreting T cells from spleens of immunized mice. Specifically, spleens were harvested from mice immunized with the SARS-CoV-2 universal mRNA vaccine, and dissociated into single cells using the gentleMACS instrument in RPMI medium containing 10% FBS and 1% Penicillin-Streptomycin. The splenocytes were filtered through a 70 μm strainer and centrifuged at 2,000 rpm for 5 minutes at 4° C. After removing supernatant, remained pellets were suspended in 5 ml of ACK lysing buffer, vortexed, and incubated at 37° C. for 5 minutes. An additional 10 ml of RPMI medium containing 10% FBS and 1% Penicillin-Streptomycin was added, followed by centrifugation under the same conditions. The supernatant was discarded, and the pellet was resuspended in 1-3 ml of RPMI medium containing 10% FBS and 1% Penicillin-Streptomycin. Splenocyte counts were determined using a LUNA cell counter. Splenocytes were seeded at 2.5×10⁵ cells/well in a 96-well plate in a Mouse IFN-γ ELISpot Plus Kit (R&D Systems). SARS-CoV-2 peptides were diluted to a final concentration of 1 μg/well, and added as stimulants. Mixtures of the cells and stimulants were incubated at 37° C. with 5% CO₂ for 12-48 hours. After incubation, the mixtures were removed, and the wells were washed with PBS. Primary antibodies diluted in PBS containing 0.5% FBS were added, and incubated at ambient temperature for 2 hours. After washing, ALP-conjugated secondary antibodies diluted in PBS with 0.5% FBS were added, and incubated for 1 hour at ambient temperature. Following another wash, a sterile-filtered substrate solution (BCIP/NBT-plus) was added, and allowed to react for 10-30 minutes until spots appeared. The reaction was stopped by washing with distilled water, and the plates were thoroughly dried. The resulting spots were counted using a CTL automated reader.

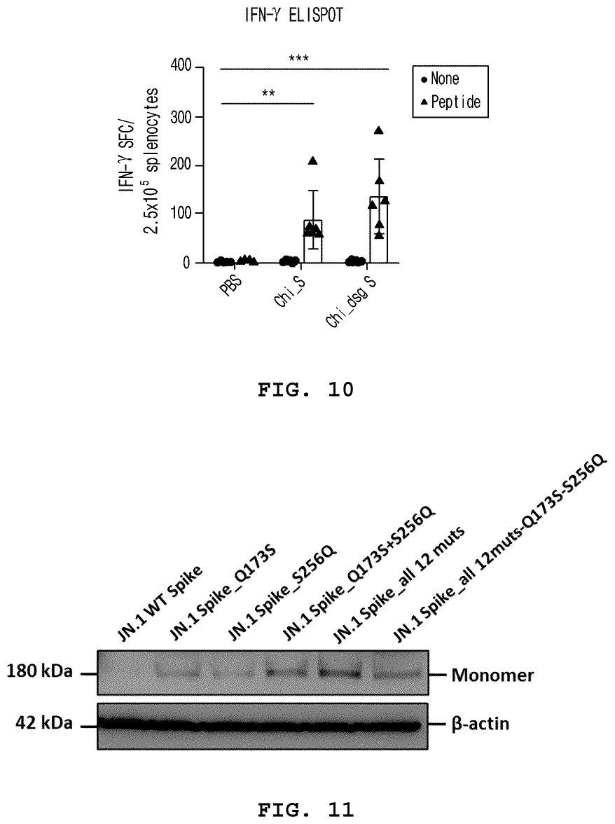

As a result of this experiment, it was confirmed that the SARS-CoV-2 Omicron universal mRNA vaccine of the present invention (denoted as “Chimeric_dsg S” or “Chi_dsg S” in FIG. 10) demonstrated significantly superior cellular immune responses compared to the Omicron Chimeric Spike vaccine (denoted as “Chimeric S” or “Chi S” in FIG. 10).

### [Example 6] Verification of the Protein Stabilization Effect of Common Mutations in the SARS-CoV-2 Universal mRNA Vaccine

### <Example 6-1>Construction of JN.1 Variant Spike Mutants

To validate that the Q173 and S256 positions, which are commonly observed in the SARS-CoV-2 universal spike antigens of SEQ ID NOS: 2 and 3 prepared in Preparative Example 3, represent universal sites contributing to spike protein stabilization, variant spike constructs were generated based on the recently reported JN.1 spike protein sequence.

Specifically, the following constructs were generated: (1) JN.1 wild-type spike (JN.1 S), (2) JN.1 S+Q173S mutation, (3) JN.1 S+S256Q mutation, (4) JN.1 S+Q173S and S256Q double mutations, (5) JN.1 S containing all 12 stabilization mutations, and (6) JN.1 S containing the 12 stabilization mutations except for Q173S and S256Q.

The DNA sequences of the generated spike variants are presented in Table 9, and the amino acid sequences of the expressed spike proteins are shown in Table 10.

TABLE 9
DNA sequences of JN.1 variant spike mutants

DNA corresponding to SEQ ID NO: 14 (JN.1 wild-type spike (JN.1 S))
(5′ → 3′) (SEQ ID NO: 14
ATGTTTGTTTTTCTTGTTTTATTGCCACTAGTCTCTAGTCAGTGTGTCATGCCGCTGTTTAATCTTAT
AACTACAACTCAATCATACACTAATTCTTTCACACGTGGTGTTTATTACCCTGACAAAGTTTTCAGAT
CCTCAGTTTTACATTTAACTCAGGACTTGTTCTTACCTTTCTTTTCCAATGTTACTTGGTTCCATGCT
ATCTCTGGGACCAATGGTACTAAGAGGTTTGATAACCCTGTCCTACCATTTAATGATGGTGTTTATTT
TGCTTCCACTGAGAAGTCTAACATAATAAGAGGCTGGATTTTTGGTACTACTTTAGATTCGAAGACCC
AGTCCCTACTTATTGTTAATAACGCTACTAATGTTTTTATTAAAGTCTGTGAATTTCAATTTTGTAAT
GATCCATTTTTGGGTGTTTACCACAAAAACAACAAAAGTTGGATGGAAAGTGAGTCAGGAGTTTATTC
TAGTGCGAATAATTGCACTTTTGAATATGTCTCTCAGCCTTTTCTTATGGACCTTGAAGGAAAACAGG
GTAATTTCAAAAATCTTAGGGAATTTGTGTTTAAGAATATTGATGGTTATTTTAAAATATATTCTAAG
CACACGCCTATTATAGGGCGTGATTTCCCTCAGGGTTTTTCGGCTTTAGAACCATTGGTAGATTTGCC
AATAGGTATTAACATCACTAGGTTTCAAACTTTACTTGCTTTAAATAGAAGTTATTTGACTCCTGGTG
ATTCTTCTTCAGGTTGGACAGCTGGTGCTGCAGATTATTATGTGGGTTATCTTCAACCTAGGACTTTT
CTATTAAAATATAATGAAAATGGAACCATTACAGATGCTGTAGACTGTGCACTTGACCCTCTCTCAGA
AACAAAGTGTACGTTGAAATCCTTCACTGTAGAAAAAGGAATCTATCAAACTTCTAACTTTAGAGTCC
AACCAACAGAATCTATTGTTAGATTTCCTAATGTTACAAACTTGTGCCCTTTTCATGAAGTTTTTAAC
GCCACCAGATTTGCATCTGTTTATGCTTGGAACAGGACGAGAATCAGCAACTGTGTTGCTGATTATTC
TGTCCTATATAATTTCGCACCATTTTTCGCTTTTAAGTGTTATGGAGTGTCTCCTACTAAATTAAATG
ATCTCTGCTTTACTAATGTCTATGCAGATTCATTTGTAATTAAAGGTAATGAAGTCAGCCAAATCGCT
CCAGGGCAAACTGGAAATATTGCTGATTATAATTATAAATTACCAGATGATTTTACAGGCTGCGTTAT
AGCTTGGAATTCTAACAAGCTTGATTCTAAGCATAGTGGTAATTATGATTACTGGTATAGATCGTTTA
GGAAGTCTAAACTCAAACCTTTTGAGAGAGATATTTCAACTGAAATCTATCAGGCCGGTAACAAACCT
TGTAAAGGTAAAGGTCCTAATTGTTACTTTCCTTTACAATCATATGGTTTCCGACCCACTTATGGTGT
TGGTCACCAACCATACAGAGTAGTAGTACTTTCTTTTGAACTTCTACATGCACCAGCAACTGTTTGTG
GACCTAAAAAGTCTACTAATTTGGTTAAAAACAAATGTGTCAATTTCAACTTCAATGGTTTAACAGGC
ACAGGTGTTCTTACTAAGTCTAACAAAAAGTTTCTGCCTTTCCAACAATTTGGCAGAGACATTGTTGA
CACTACTGATGCTGTCCGTGATCCACAGACACTTGAGATTCTTGACATTACACCATGTTCTTTTGGTG
GTGTCAGTGTTATAACACCAGGAACAAATACTTCTAACCAGGTTGCTGTTCTTTATCAGGGTGTTAAC
TGCACAGAAGTCTCTGTTGCTATTCATGCAGATCAACTTACTCCTACTTGGCGTGTTTATTCTACAGG
TTCTAATGTTTTTCAAACACGTGCAGGCTGTTTAATAGGGGCTGAATATGTCAACAACTCATATGAGT
GTGACATACCCATTGGTGCAGGTATATGCGCTAGTTATCAGACTCAGACTAAGTCTCGTCGGCGGGCA
CGTAGTGTAGCTAGTCAATCCATCATTGCCTACACTATGTCACTTGGTGCAGAAAATTCAGTTGCTTA
CTCTAATAACTCTATTGCCATACCCACAAATTTTACTATTAGTGTTACCACAGAAATTCTACCAGTGT
CTATGACCAAGACATCAGTAGATTGTACAATGTACATTTGTGGTGATTCAACTGAATGCAGCAATCTT
TTGTTGCAATATGGCAGTTTTTGTACACAATTAAAACGTGCTTTAACTGGAATAGCTGTTGAACAAGA
CAAAAACACCCAAGAAGTTTTTGCACAAGTCAAACAAATTTACAAAACACCACCAATTAAATATTTTG
GTGGTTTTAATTTTTCACAAATATTACCAGATCCATCAAAACCAAGCAAGAGGTCATTTATTGAAGAT
CTACTTTTCAACAAAGTGACACTTGCAGATGCTGGCTTCATCAAACAATATGGTGATTGCCTTGGTGA
TATTGCTGCTAGAGACCTCATTTGTGCACAAAAGTTTAACGGCCTTACTGTTTTGCCACCTTTGCTCA
CAGATGAAATGATTGCTCAATACACTTCTGCACTGTTAGCGGGTACAATCACTTCTGGTTGGACCTTT
GGTGCAGGTGCTGCATTACAAATACCATTTGCTATGCAAATGGCTTATAGGTTTAATGGTATTGGAGT
TACACAGAATGTTCTCTATGAGAACCAAAAATTGATTGCCAACCAATTTAATAGTGCTATTGGCAAAA
TTCAAGACTCACTTTTTTCCACAGCAAGTGCACTTGGAAAACTTCAAGATGTGGTCAACCATAATGCA
CAAGCTTTAAACACGCTTGTTAAACAACTTAGCTCCAAATTTGGTGCAATTTCAAGTGTTTTAAATGA
TATCCTTTCACGTCTTGACAAAGTTGAGGCTGAAGTGCAAATTGATAGGTTGATCACAGGCAGACTTC
AAAGTTTGCAGACATATGTGACTCAACAATTAATTAGAGCTGCAGAAATCAGAGCTTCTGCTAATCTT
GCTGCTACTAAAATGTCAGAGTGTGTACTTGGACAATCAAAAAGAGTTGATTTTTGTGGAAAGGGCTA
TCATCTTATGTCCTTCCCTCAGTCAGCACCTCATGGTGTAGTCTTCTTGCATGTGACTTATGTCCCTG
CACAAGAAAAGAACTTCACAACTGCTCCTGCCATTTGTCATGATGGAAAAGCACACTTTCCTCGTGAA
GGTGTCTTTGTTTCAAATGGCACACACTGGTTTGTAACACAAAGGAATTTTTATGAACCACAAATCAT
TACTACAGACAACACATTTGTGTCTGGTAACTGTGATGTTGTAATAGGAATTGTCAACAACACAGTTT
ATGATCCTTTGCAACTTGAATTAGATTCATTCAAGGAGGAGTTAGATAAATATTTTAAGAATCATACA
TCACCAGATGTTGATTTAGGTGACATCTCTGGCATTAATGCTTCAGTTGTAAACATTCAAAAAGAAAT
TGACCGCCTCAATGAGGTTGCCAAGAATTTAAATGAATCTCTCATCGATCTCCAAGAACTTGGAAAGT
ATGAGCAGTATATAAAATGGCCATGGTATATTTGGCTAGGTTTTATAGCTGGCTTGATTGCCATAGTA
ATGGTGACAATTATGCTTTGCTGTATGACCAGTTGCTGTAGTTGTCTCAAGGGCTGTTGTTCTTGTGG
ATCCTGCTGCAAATTTGATGAAGACGACTCTGAGCCAGTGCTCAAAGGAGTCAAATTACATTACACAT
AA
DNA corresponding to SEQ ID NO: 15 (JN.1 S + Q173S mutation) (5′ → 3′)
(SEQ ID NO: 15)
ATGTTTGTGTTCCTCGTATTACTTCCGCTGGTGTCGAGCCAGTGCGTGATGCCGTTGTTCAACCTGAT
CACGACAACACAGAGCTACACGAACAGCTTTACTCGGGGTGTGTATTACCCCGATAAAGTGTTCCGTA
GCTCTGTGTTGCATCTGACTCAGGACCTCTTCCTCCCGTTCTTCTCCAATGTCACGTGGTTCCACGCG
ATATCGGGGACGAACGGGACGAAGAGGTTCGACAACCCTGTTCTGCCGTTCAACGACGGGGTGTACTT
CGCTTCGACAGAGAAGTCCAACATCATCCGGGGCTGGATCTTCGGTACCACCCTAGATAGTAAGACGC
AGTCCCTGCTGATCGTGAACAACGCTACTAACGTGTTCATCAAGGTGTGTGAGTTCCAGTTCTGTAAC
GACCCCTTTCTCGGAGTCTATCACAAGAACAACAAGTCTTGGATGGAGTCCGAGAGGGGGGTCTACAG
TTCTGCGAACAACTGCACCTTTGAATACGTTAGTAGCCCGTTCTTGATGGATCTCGAGGGGAAGCAGG
GCAACTTTAAAAACCTGCGGGAATTCGTGTTCAAGAATATCGACGGGTACTICAAGATCTACTCGAAG
CATACCCCGATAATTGGACGCGATTTCCCGCAGGGGTTTAGTGCCCTGGAGCCCCTCGTAGATCTTCC
AATCGGAATTAATATCACCCGGTTTCAGACGCTCCTGGCGCTGAACAGGAGCTATCTGACGCCGGGTG
ATAGTAGTTCCGGTTGGACAGCAGGGGCTGCGGATTACTATGTAGGGTACCTCCAGCCCCGGACGTTC
TTGTTGAAGTACAACGAGAACGGCACAATCACCGACGCGGTTGATTGTGCGTTGGACCCTCTGTCGGA
GACGAAGTGCACCCTGAAGTCGTTTACGGTAGAAAAGGGGATCTATCAGACCTCCAACTTCCGCGTCC
AGCCGACTGAGAGTATCGTTCGGTTCCCGAACGTTACTAATCTGTGTCCGTTCCACGAGGTATTCAAC
GCTACGCGGTTCGCGAGCGTGTACGCGTGGAACCGGACACGGATTAGTAACTGTGTAGCAGACTACAG
TGTGCTATACAACTTCGCCCCGTTCTTCGCGTTCAAGTGTTACGGGGTGTCGCCCACGAAGTTGAACG
ACCTCTGCTTCACCAACGTGTACGCCGACAGCTTTGTGATTAAGGGCAACGAGGTCTCGCAGATCGCC
CCAGGGCAGACGGGGAACATTGCTGACTACAACTACAAGCTGCCTGACGATTTTACCGGTTGCGTTAT
TGCCTGGAATAGCAATAAGCTTGACAGCAAGCATTCCGGAAACTACGATTACTGGTATCGTAGTTTCC
GGAAGAGCAAGCTGAAGCCCTTCGAGAGGGATATCTCTACGGAGATATACCAGGCCGGAAACAAGCCC
TGCAAGGGGAAGGGGCCGAACTGCTACTTCCCCTTGCAGAGCTACGGTTTCCGGCCTACCTACGGGGT
AGGGCACCAGCCCTACCGCGTAGTGGTGCTCTCCTTCGAGCTGCTCCACGCACCGGCGACGGTGTGTG
GGCCGAAGAAGAGCACCAATCTCGTGAAGAACAAATGCGTCAACTTCAATTTCAATGGACTGACTGGA
ACGGGAGTGCTCACGAAGAGCAACAAGAAGTTCCTCCCGTTCCAGCAGTTCGGGCGTGATATCGTTGA
CACCACCGATGCTGTGCGTGATCCCCAGACTCTGGAGATTCTGGATATCACGCCGTGCAGCTTCGGTG
GTGTCAGCGTTATCACGCCCGGAACGAATACATCCAACCAGGTGGCGGTGCTGTATCAGGGCGTGAAT
TGCACCGAGGTATCGGTTGCAATTCACGCCGATCAGCTGACCCCCACCTGGCGGGTGTATTCCACGGG
CAGCAATGTCTTCCAGACACGCGCCGGCTGCTTGATAGGGGCAGAGTATGTGAATAATAGCTACGAGT
GCGACATTCCGATCGGCGCCGGGATTTGTGCGTCGTACCAGACGCAGACCAAGTCCCGGCGCCGAGCT
CGGAGTGTCGCCTCGCAGTCTATTATTGCATATACTATGTCCCTGGGAGCCGAGAACTCAGTAGCATA
TTCGAACAACTCCATTGCTATCCCCACAAATTTTACAATTAGTGTAACCACCGAGATCTTGCCGGTCT
CGATGACCAAGACCTCGGTGGATTGCACTATGTATATTTGTGGGGATAGCACGGAGTGTTCGAATTTG
CTGCTGCAGTACGGCTCCTTCTGCACTCAGCTGAAGCGAGCACTGACTGGGATTGCTGTCGAGCAGGA
CAAGAATACCCAGGAGGTGTTCGCTCAGGTGAAGCAGATCTACAAGACGCCCCCGATCAAGTACTTCG
GGGGCTTCAACTTCAGCCAGATTCTGCCAGACCCATCTAAGCCGAGCAAGAGGTCCTTTATTGAGGAC
CTCTTGTTCAACAAGGTGACTCTGGCAGATGCTGGCTTCATCAAGCAGTACGGCGACTGTCTGGGAGA
CATTGCTGCCCGTGACCTCATCTGCGCGCAGAAGTTCAACGGTCTGACAGTGCTGCCCCCGCTCCTCA
CCGACGAGATGATCGCCCAGTACACCAGCGCGCTGCTGGCTGGGACGATCACCTCGGGGTGGACCTTC
GGAGCGGGGGCAGCACTGCAGATCCCTTTTGCGATGCAGATGGCGTATCGGTTCAATGGAATTGGAGT
GACGCAGAATGTTCTTTACGAGAACCAGAAGCTTATTGCTAACCAGTTCAATAGCGCAATCGGTAAGA
TCCAGGACAGCTTGTTCAGCACCGCGTCTGCCCTGGGGAAGCTGCAAGACGTCGTTAATCACAATGCT
CAGGCGTTGAACACGTTGGTGAAGCAGTTGTCGTCCAAGTTCGGGGCGATCAGTTCGGTGCTGAACGA
TATTCTCAGTCGGCTGGACAAGGTGGAAGCGGAGGTCCAGATAGATCGGCTCATCACTGGTCGCCTCC
AGAGTTTGCAGACGTACGTAACTCAGCAGCTCATCCGAGCTGCTGAGATACGTGCGTCTGCAAACCTG
GCGGCGACCAAGATGAGCGAGTGCGTGCTTGGACAGTCCAAGCGCGTAGACTTCTGCGGGAAGGGCTA
TCACCTGATGTCCTTCCCGCAGAGCGCCCCCCACGGGGTGGTCTTCCTGCACGTGACATATGTGCCGG
CGCAGGAGAAGAACTTCACGACTGCGCCGGCCATATGTCACGACGGGAAGGCCCACTTCCCCCGTGAG
GGGGTGTTCGTGAGCAACGGCACGCACTGGTTCGTCACCCAGCGGAACTTTTACGAGCCACAGATAAT
TACCACTGACAATACCTTTGTCAGTGGTAACTGCGACGTGGTCATAGGCATTGTGAACAACACTGTCT
ATGACCCGTTGCAGTTGGAGCTTGACTCCTTTAAGGAGGAGCTCGACAAGTACTTCAAGAATCATACC
TCGCCGGACGTTGATCTCGGAGACATCTCCGGGATCAACGCTTCGGTGGTCAATATCCAGAAGGAGAT
TGACCGCCTCAACGAGGTGGCCAAGAACCTTAATGAATCGCTCATAGATCTCCAGGAGCTGGGGAAGT
ATGAGCAGTACATTAAGTGGCCTTGGTACATCTGGTTGGGGTTTATAGCAGGGCTGATCGCGATCGTG
ATGGTCACGATCATGCTCTGCTGTATGACGAGCTGCTGCAGCTGCCTCAAGGGCTGCTGCTCTTGTGG
CAGCTGCTGCAAGTTCGACGAGGATGATTCCGAGCCCGTCCTTAAAGGAGTCAAGCTCCACTACACG
DNA corresponding to SEQ ID NO: 16 (JN.1 S + S256Q mutation)
(5′ → 3′) (SEQ ID NO: 16)
ATGTTTGTGTTCCTCGTATTACTTCCGCTGGTGTCGAGCCAGTGCGTGATGCCGTTGTTCAACCTGAT
CACGACAACACAGAGCTACACGAACAGCTTTACTCGGGGTGTGTATTACCCCGATAAAGTGTTCCGTA
GCTCTGTGTTGCATCTGACTCAGGACCTCTTCCTCCCGTTCTTCTCCAATGTCACGTGGTTCCACGCG
ATATCGGGGACGAACGGGACGAAGAGGTTCGACAACCCTGTTCTGCCGTTCAACGACGGGGTGTACTT
CGCTTCGACAGAGAAGTCCAACATCATCCGGGGCTGGATCTTCGGTACCACCCTAGATAGTAAGACGC
AGTCCCTGCTGATCGTCAATAACGCGACTAACGTGTTCATCAAGGTGTGTGAGTTCCAGTTCTGTAAC
GACCCCTTTCTCGGAGTCTATCACAAGAACAACAAGTCTTGGATGGAGTCCGAGAGCGGGGTCTACAG
TTCTGCGAACAACTGCACCTTTGAATACGTTAGTCAGCCGTTTTTGATGGACCTCGAGGGGAAGCAGG
GCAACTTTAAAAACCTGCGGGAATTCGTGTTCAAGAATATCGACGGGTACTTCAAGATCTACTCGAAG
CATACCCCGATAATTGGACGCGATTTCCCGCAGGGGTTTAGTGCCCTGGAGCCCCTCGTGGATCTACC
CATTGGGATCAATATCACCCGGTTTCAGACGCTCCTGGCGCTGAACAGGAGCTATCTGACGCCGGGTG
ATAGTTCCCAAGGGTGGACAGCAGGGGCTGCGGATTACTATGTAGGGTACCTCCAGCCCCGGACGTTC
TTGTTGAAGTACAACGAGAACGGCACAATCACCGACGCGGTTGATTGTGCGTTGGACCCTCTGTCGGA
GACGAAGTGCACCCTGAAGTCGTTTACGGTAGAAAAGGGGATCTATCAGACCTCCAACTTCCGCGTCC
AGCCGACTGAGAGTATCGTTCGGTTCCCGAACGTTACTAATCTGTGTCCGTTCCACGAGGTATTCAAC
GCTACGCGGTTCGCGAGCGTGTACGCGTGGAACCGGACACGGATTAGTAACTGTGTAGCAGACTACAG
TGTGCTATACAACTTCGCCCCGTTCTTCGCGTTCAAGTGTTACGGGGTGTCGCCCACGAAGTIGAACG
ACCTCTGCTTCACCAACGTGTACGCCGACAGCTTTGTGATTAAGGGCAACGAGGTCTCGCAGATCGCC
CCAGGGCAGACGGGGAACATTGCTGACTACAACTACAAGCTGCCTGACGATTTTACCGGTTGCGTTAT
TGCCTGGAATAGCAATAAGCTTGACAGCAAGCATTCCGGAAACTACGATTACTGGTATCGTAGTTTCC
GGAAGAGCAAGCTGAAGCCCTTCGAGAGGGATATCTCTACGGAGATATACCAGGCCGGAAACAAGCCC
TGCAAGGGGAAGGGGCCGAACTGCTACTTCCCCTTGCAGAGCTACGGTTTCCGGCCTACCTACGGGGT
AGGGCACCAGCCCTACCGCGTAGTGGTGCTCTCCTTCGAGCTGCTCCACGCACCGGCGACGGTGTGTG
GGCCGAAGAAGAGCACCAATCTCGTGAAGAACAAATGCGTCAACTTCAATTTCAATGGACTGACTGGA
ACGGGAGTGCTCACGAAGAGCAACAAGAAGTTCCTCCCGTTCCAGCAGTTCGGGCGTGATATCGTTGA
CACCACCGATGCTGTGCGTGATCCCCAGACTCTGGAGATTCTGGATATCACGCCGTGCAGCTTCGGTG
GTGTCAGCGTTATCACGCCCGGAACGAATACATCCAACCAGGTGGCGGTGCTGTATCAGGGCGTGAAT
TGCACCGAGGTATCGGTTGCAATTCACGCCGATCAGCTGACCCCCACCTGGCGGGTGTATTCCACGGG
CAGCAATGTCTTCCAGACACGCGCCGGCTGCTTGATAGGGGCAGAGTATGTGAATAATAGCTACGAGT
GCGACATTCCGATCGGCGCCGGGATTTGTGCGTCGTACCAGACGCAGACCAAGTCCCGGCGCCGAGCT
CGGAGTGTCGCCTCGCAGTCTATTATTGCATATACTATGTCCCTGGGAGCCGAGAACTCAGTAGCATA
TTCGAACAACTCCATTGCTATCCCCACAAATTTTACAATTAGTGTAACCACCGAGATCTTGCCGGTCT
CGATGACCAAGACCTCGGTGGATTGCACTATGTATATTTGTGGGGATAGCACGGAGTGTTCGAATTTG
CTGCTGCAGTACGGCTCCTTCTGCACTCAGCTGAAGCGAGCACTGACTGGGATTGCTGTCGAGCAGGA
CAAGAATACCCAGGAGGTGTTCGCTCAGGTGAAGCAGATCTACAAGACGCCCCCGATCAAGTACTTCG
GGGGCTTCAACTTCAGCCAGATTCTGCCAGACCCATCTAAGCCGAGCAAGAGGTCCTTTATTGAGGAC
CTCTTGTTCAACAAGGTGACTCTGGCAGATGCTGGCTTCATCAAGCAGTACGGCGACTGTCTGGGAGA
CATTGCTGCCCGTGACCTCATCTGCGCGCAGAAGTTCAACGGTCTGACAGTGCTGCCCCCGCTCCTCA
CCGACGAGATGATCGCCCAGTACACCAGCGCGCTGCTGGCTGGGACGATCACCTCGGGGTGGACCTTC
GGAGCGGGGGCAGCACTGCAGATCCCTTTTGCGATGCAGATGGCGTATCGGTTCAATGGAATTGGAGT
GACGCAGAATGTTCTTTACGAGAACCAGAAGCTTATTGCTAACCAGTTCAATAGCGCAATCGGTAAGA
TCCAGGACAGCTTGTTCAGCACCGCGTCTGCCCTGGGGAAGCTGCAAGACGTCGTTAATCACAATGCT
CAGGCGTTGAACACGTTGGTGAAGCAGTTGTCGTCCAAGTTCGGGGCGATCAGTTCGGTGCTGAACGA
TATTCTCAGTCGGCTGGACAAGGTGGAAGCGGAGGTCCAGATAGATCGGCTCATCACTGGTCGCCTCC
AGAGTTTGCAGACGTACGTAACTCAGCAGCTCATCCGAGCTGCTGAGATACGTGCGTCTGCAAACCTG
GCGGCGACCAAGATGAGCGAGTGCGTGCTTGGACAGTCCAAGCGCGTAGACTTCTGCGGGAAGGGCTA
TCACCTGATGTCCTTCCCGCAGAGCGCCCCCCACGGGGTGGTCTTCCTGCACGTGACATATGTGCCGG
CGCAGGAGAAGAACTTCACGACTGCGCCGGCCATATGTCACGACGGGAAGGCCCACTTCCCCCGTGAG
GGGGTGTTCGTGAGCAACGGCACGCACTGGTTCGTCACCCAGCGGAACTTTTACGAGCCACAGATAAT
TACCACTGACAATACCTTTGTCAGTGGTAACTGCGACGTGGTCATAGGCATTGTGAACAACACTGTCT
ATGACCCGTTGCAGTTGGAGCTTGACTCCTTTAAGGAGGAGCTCGACAAGTACTTCAAGAATCATACC
TCGCCGGACGTTGATCTCGGAGACATCTCCGGGATCAACGCTTCGGTGGTCAATATCCAGAAGGAGAT
TGACCGCCTCAACGAGGTGGCCAAGAACCTTAATGAATCGCTCATAGATCTCCAGGAGCTGGGGAAGT
ATGAGCAGTACATTAAGTGGCCTTGGTACATCTGGTTGGGGTTTATAGCAGGGCTGATCGCGATCGTG
ATGGTCACGATCATGCTCTGCTGTATGACGAGCTGCTGCAGCTGCCTCAAGGGCTGCTGCTCTTGTGG
CAGCTGCTGCAAGTTCGACGAGGATGATTCCGAGCCCGTCCTTAAAGGAGTCAAGCTCCACTACACG
DNA corresponding to SEQ ID NO: 17 (JN.1 S + Q173S and S256Q double
mutations) (5′ → 3′) (SEQ ID NO: 17)
ATGTTTGTGTTCCTCGTATTACTTCCGCTGGTGTCGAGCCAGTGCGTGATGCCGTTGTTCAACCTGAT
CACGACAACACAGAGCTACACGAACAGCTTTACTCGGGGTGTGTATTACCCCGATAAAGTGTTCCGTA
GCTCTGTGTTGCATCTGACTCAGGACCTCTTCCTCCCGTTCTTCTCCAATGTCACGTGGTTCCACGCG
ATATCGGGGACGAACGGGACGAAGAGGTTCGACAACCCTGTTCTGCCGTTCAACGACGGGGTGTACTT
CGCTTCGACAGAGAAGTCCAACATCATCCGGGGCTGGATCTTCGGTACCACCCTAGATAGTAAGACGC
AGTCCCTGCTGATCGTGAACAACGCTACTAACGTGTTCATCAAGGTGTGTGAGTTCCAGTTCTGTAAC
GACCCCTTTCTCGGAGTCTATCACAAGAACAACAAGTCTTGGATGGAGTCCGAGAGCGGGGTCTACAG
TTCTGCGAACAACTGCACCTTTGAATACGTTAGTAGCCCGTTCTTGATGGACCTGGAGGGCAAGCAGG
GGAACTTCAAGAACCTGCGGGAATTCGTGTTCAAGAATATCGACGGGTACTICAAGATCTACTCGAAG
CATACCCCGATAATTGGACGCGATTTCCCGCAGGGGTTCTCCGCGCTGGAGCCGCTGGTGGACTTGCC
GATAGGCATCAACATCACGCGGTTCCAGACGCTGCTTGCCCTCAACAGGTCCTATCTCACCCCTGGGG
ACTCTTCCCAGGGGTGGACAGCAGGGGCTGCGGATTACTATGTAGGGTACCTCCAGCCCCGGACGTTC
TTGTTGAAGTACAACGAGAACGGCACAATCACCGACGCGGTTGATTGTGCGTTGGACCCTCTGTCGGA
GACGAAGTGCACCCTGAAGTCGTTTACGGTAGAAAAGGGGATCTATCAGACCTCCAACTTCCGCGTCC
AGCCGACTGAGAGTATCGTTCGGTTCCCGAACGTTACTAATCTGTGTCCGTTCCACGAGGTATTCAAC
GCTACGCGGTTCGCGAGCGTGTACGCGTGGAACCGGACACGGATTAGTAACTGTGTAGCAGACTACAG
TGTGCTATACAACTTCGCCCCGTTCTTCGCGTTCAAGTGTTACGGGGTGTCGCCCACGAAGTIGAACG
ACCTCTGCTTCACCAACGTGTACGCCGACAGCTTTGTGATTAAGGGCAACGAGGTCTCGCAGATCGCC
CCAGGGCAGACGGGGAACATTGCTGACTACAACTACAAGCTGCCTGACGATTTTACCGGTTGCGTTAT
TGCCTGGAATAGCAATAAGCTTGACAGCAAGCATTCCGGAAACTACGATTACTGGTATCGTAGTTTCC
GGAAGAGCAAGCTGAAGCCCTTCGAGAGGGATATCTCTACGGAGATATACCAGGCCGGAAACAAGCCC
TGCAAGGGGAAGGGGCCGAACTGCTACTTCCCCTTGCAGAGCTACGGTTTCCGGCCTACCTACGGGGT
AGGGCACCAGCCCTACCGCGTAGTGGTGCTCTCCTTCGAGCTGCTCCACGCACCGGCGACGGTGTGTG
GGCCGAAGAAGAGCACCAATCTCGTGAAGAACAAATGCGTCAACTTCAATTTCAATGGACTGACTGGA
ACGGGAGTGCTCACGAAGAGCAACAAGAAGTTCCTCCCGTTCCAGCAGTTCGGGCGTGATATCGTTGA
CACCACCGATGCTGTGCGTGATCCCCAGACTCTGGAGATTCTGGATATCACGCCGTGCAGCTTCGGTG
GTGTCAGCGTTATCACGCCCGGAACGAATACATCCAACCAGGTGGCGGTGCTGTATCAGGGCGTGAAT
TGCACCGAGGTATCGGTTGCAATTCACGCCGATCAGCTGACCCCCACCTGGCGGGTGTATTCCACGGG
CAGCAATGTCTTCCAGACACGCGCCGGCTGCTTGATAGGGGCAGAGTATGTGAATAATAGCTACGAGT
GCGACATTCCGATCGGCGCCGGGATTTGTGCGTCGTACCAGACGCAGACCAAGTCCCGGCGCCGAGCT
CGGAGTGTCGCCTCGCAGTCTATTATTGCATATACTATGTCCCTGGGAGCCGAGAACTCAGTAGCATA
TTCGAACAACTCCATTGCTATCCCCACAAATTTTACAATTAGTGTAACCACCGAGATCTTGCCGGTCT
CGATGACCAAGACCTCGGTGGATTGCACTATGTATATTTGTGGGGATAGCACGGAGTGTTCGAATTTG
CTGCTGCAGTACGGCTCCTTCTGCACTCAGCTGAAGCGAGCACTGACTGGGATTGCTGTCGAGCAGGA
CAAGAATACCCAGGAGGTGTTCGCTCAGGTGAAGCAGATCTACAAGACGCCCCCGATCAAGTACTTCG
GGGGCTTCAACTTCAGCCAGATTCTGCCAGACCCATCTAAGCCGAGCAAGAGGTCCTTTATTGAGGAC
CTCTTGTTCAACAAGGTGACTCTGGCAGATGCTGGCTTCATCAAGCAGTACGGCGACTGTCTGGGAGA
CATTGCTGCCCGTGACCTCATCTGCGCGCAGAAGTTCAACGGTCTGACAGTGCTGCCCCCGCTCCTCA
CCGACGAGATGATCGCCCAGTACACCAGCGCGCTGCTGGCTGGGACGATCACCTCGGGGTGGACCTTC
GGAGCGGGGGCAGCACTGCAGATCCCTTTTGCGATGCAGATGGCGTATCGGTTCAATGGAATTGGAGT
GACGCAGAATGTTCTTTACGAGAACCAGAAGCTTATTGCTAACCAGTTCAATAGCGCAATCGGTAAGA
TCCAGGACAGCTTGTTCAGCACCGCGTCTGCCCTGGGGAAGCTGCAAGACGTCGTTAATCACAATGCT
CAGGCGTTGAACACGTTGGTGAAGCAGTTGTCGTCCAAGTTCGGGGCGATCAGTTCGGTGCTGAACGA
TATTCTCAGTCGGCTGGACAAGGTGGAAGCGGAGGTCCAGATAGATCGGCTCATCACTGGTCGCCTCC
AGAGTTTGCAGACGTACGTAACTCAGCAGCTCATCCGAGCTGCTGAGATACGTGCGTCTGCAAACCTG
GCGGCGACCAAGATGAGCGAGTGCGTGCTTGGACAGTCCAAGCGCGTAGACTTCTGCGGGAAGGGCTA
TCACCTGATGTCCTTCCCGCAGAGCGCCCCCCACGGGGTGGTCTTCCTGCACGTGACATATGTGCCGG
CGCAGGAGAAGAACTTCACGACTGCGCCGGCCATATGTCACGACGGGAAGGCCCACTTCCCCCGTGAG
GGGGTGTTCGTGAGCAACGGCACGCACTGGTTCGTCACCCAGCGGAACTTTTACGAGCCACAGATAAT
TACCACTGACAATACCTTTGTCAGTGGTAACTGCGACGTGGTCATAGGCATTGTGAACAACACTGTCT
ATGACCCGTTGCAGTTGGAGCTTGACTCCTTTAAGGAGGAGCTCGACAAGTACTTCAAGAATCATACC
TCGCCGGACGTTGATCTCGGAGACATCTCCGGGATCAACGCTTCGGTGGTCAATATCCAGAAGGAGAT
TGACCGCCTCAACGAGGTGGCCAAGAACCTTAATGAATCGCTCATAGATCTCCAGGAGCTGGGGAAGT
ATGAGCAGTACATTAAGTGGCCTTGGTACATCTGGTTGGGGTTTATAGCAGGGCTGATCGCGATCGTG
ATGGTCACGATCATGCTCTGCTGTATGACGAGCTGCTGCAGCTGCCTCAAGGGCTGCTGCTCTTGTGG
CAGCTGCTGCAAGTTCGACGAGGATGATTCCGAGCCCGTCCTTAAAGGAGTCAAGCTCCACTACACG
DNA corresponding to SEQ ID NO: 18 (JN.1 S containing all 12
stabilization mutations) (5′ → 3′) (SEQ ID NO: 18)
ATGTTTGTGTTCCTCGTATTACTTCCGCTGGTGTCGAGCCAGTGCGTGATGCCGTTGTTCAACTTGAT
CACGACGACTCAGGAGTACACTAATAGTTTCACTCGGGGGGTCTACTACCCGGATAAAGTATTTCGGT
CGTCAGTGCTGCACATGACGACCGATTTATTTCTTCCGTTCTTCTCCAACGTGACGTGGTTTCACGCC
ATCAGCGGGAGCAACGGGACGAAGTACTTCGACAATCCCGTGCTCCCGTTCAATGATGGCGTGTACTT
CGCGTCAACGGAGAAGAGCAACATCATCCGGGGCTGGATCTTCGGTACCACCCTAGATAGTAAGACGC
AGTCCCTGCTGATCGTGAACAACGCTACTAACGTGTTCATCAAGGTGTGTTATTTCCAGTTTTGTAAC
GACCCCTTTCTCGGAGTCTATCACAAGAACAACAAGTCTTGGATGGAGTCCGAGAGCGGGGTCTACGA
TTCTGCGAATAACTGCACCTTTGAATACGTTAGTAGCCCGTTCTTGATGGACCTGGAGGGCAAGCAGG
GGAACTTCAAGAACCTGCGTGAGTTTGTGTTCAAGAACATCGATGGGTACTICAAGATCTACTCGAAG
CACACGCCGATAATCACGCGGGACTTCCCGCAGGGATTTTCGGCGCTGGAGCCTCTGGTAGATCTGCC
CATCGGCATAAACATCACGCGGTTCCAGACCCTGCTTGCCCTCAACAGGTCCTATCTCACCCCTGGGG
ACTCTTCCCAGGGGTGGACAGTAGGGGCTGCGGATTACTATGTAGGGTACCTCCAGCCCCGGACGTTC
TTGTTGAAGTACAACGAGAACGGCACAATCACCGACGCGGTTGATTGTGCCCTAGACCCCCTGAGTGA
GACTAAGTGTACCCTGAAGTCGTTCACGGTCGAGAAGGGGATCTATCAGACCTCCAACTTCCGCGTCC
AGCCGACTGACAGTATCGTTCGGTTCCCGAACGTTACTAATCTGTGTCCGTTCCACGAGGTATTCAAC
GCTACGCGGTTCGCGAGCGTGTACGCGTGGAACCGGACACGGATTAGTAACTGTGTAGCAGACTACAG
TGTGCTATACAACTTCGCCCCGTTCTTCGCGTTCAAGTGTTACGGGGTGTCGCCCACGAAGTTGAACG
ACCTCTGCTTCACCAACGTGTACGCCGACAGCTTTGTGATTAAGGGCAACGAGGTCTCGCAGATCGCC
CCAGGGCAGACGGGGAACATTGCTGACTACAACTACAAGCTGCCTGACGATTTTACCGGTTGCGTTAT
TGCCTGGAATAGCAATAAGCTTGACAGCAAGCATTCCGGAAACTACGATTACTGGTATCGTAGTTTCC
GGAAGAGCAAGCTGAAGCCCTTCGAGAGGGATATCTCTACGGAGATATACCAGGCCGGAAACAAGCCC
TGCAAGGGGAAGGGGCCGAACTGCTACTTCCCCTTGCAGAGCTACGGTTTCCGGCCTACCTACGGGGT
AGGGCACCAGCCCTACCGCGTAGTGGTGCTCTCCTTCGAGCTGCTCCACGCACCGGCGACGGTGTGTG
GGCCGAAGAAGAGCACCAATCTCGTGAAGAACAAATGCGTCAACTTCAATTTCAATGGACTGACTGGA
ACGGGAGTGCTCACGAAGAGCAACAAGAAGTTCCTCCCGTTCCAGCAGTTCGGGCGTGATATCGTTGA
CACCACCGATGCTGTGCGTGATCCCCAGACTCTGGAGATTCTGGATATCACGCCGTGCAGCTTCGGTG
GTGTCAGCGTTATCACGCCCGGAACGAATACATCCAACCAGGTGGCGGTGCTGTATCAGGGCGTGAAT
TGCACCGAGGTATCGGTTGCAATTCACGCCGATCAGCTGACCCCCACCTGGCGGGTGTATTCCACGGG
CAGCAATGTCTTCCAGACACGCGCCGGCTGCTTGATAGGGGCAGAGTATGTGAATAATAGCTACGAGT
GCGACATTCCGATCGGCGCCGGGATTTGTGCGTCGTACCAGACGCAGACCAAGTCCCGGCGCCGAGCT
CGGAGTGTCGCCTCGCAGTCTATTATTGCATATACTATGTCCCTGGGAGCCGAGAACTCAGTAGCATA
TTCGAACAACTCCATTGCTATCCCCACAAATTTTACAATTAGTGTAACCACCGAGATCTTGCCGGTCT
CGATGACCAAGACCTCGGTGGATTGCACTATGTATATTTGTGGGGATAGCACGGAGTGTTCGAATTTG
CTGCTGCAGTACGGCTCCTTCTGCACTCAGCTGAAGCGAGCACTGACTGGGATTGCTGTCGAGCAGGA
CAAGAATACCCAGGAGGTGTTCGCTCAGGTGAAGCAGATCTACAAGACGCCCCCGATCAAGTACTTCG
GGGGCTTCAACTTCAGCCAGATTCTGCCAGACCCATCTAAGCCGAGCAAGAGGTCCTTTATTGAGGAC
CTCTTGTTCAACAAGGTGACTCTGGCAGATGCTGGCTTCATCAAGCAGTACGGCGACTGTCTGGGAGA
CATTGCTGCCCGTGACCTCATCTGCGCGCAGAAGTTCAACGGTCTGACAGTGCTGCCCCCGCTCCTCA
CCGACGAGATGATCGCCCAGTACACCAGCGCGCTGCTGGCTGGGACGATCACCTCGGGGTGGACCTTC
GGAGCGGGGGCAGCACTGCAGATCCCTTTTGCGATGCAGATGGCGTATCGGTTCAATGGAATTGGAGT
GACGCAGAATGTTCTTTACGAGAACCAGAAGCTTATTGCTAACCAGTTCAATAGCGCAATCGGTAAGA
TCCAGGACAGCTTGTTCAGCACCGCGTCTGCCCTGGGGAAGCTGCAAGACGTCGTTAATCACAATGCT
CAGGCGTTGAACACGTTGGTGAAGCAGTTGTCGTCCAAGTTCGGGGCGATCAGTTCGGTGCTGAACGA
TATTCTCAGTCGGCTGGACAAGGTGGAAGCGGAGGTCCAGATAGATCGCCTTATCACTGGTCGCCTCC
AGAGTTTGCAGACGTACGTAACTCAGCAGCTCATCCGAGCTGCTGAGATACGTGCGTCTGCAAACCTG
GCGGCGACCAAGATGTCCGAGTGCGTGCTCGGGCAGAGCAAGCGCGTGGACTTCTGCGGGAAGGGCTA
TCACCTGATGTCCTTCCCGCAGAGCGCCCCCCACGGGGTGGTCTTCCTGCACGTGACATATGTGCCGG
CGCAGGAGAAGAACTTCACGACTGCGCCGGCCATATGTCACGACGGGAAGGCCCACTTCCCCCGTGAG
GGGGTGTTCGTGAGCAACGGCACGCACTGGTTCGTCACCCAGCGGAACTTTTACGAGCCACAGATAAT
TACCACTGACAATACCTTTGTCAGTGGTAACTGCGACGTGGTCATAGGCATTGTGAACAACACTGTCT
ATGACCCGTTGCAGTTGGAGCTTGACTCCTTTAAGGAGGAGCTCGACAAGTACTTCAAGAATCATACC
TCGCCGGACGTTGATCTCGGAGACATCTCCGGGATCAACGCTTCGGTGGTCAATATCCAGAAGGAGAT
TGACCGCCTCAACGAGGTGGCCAAGAACCTTAATGAATCGCTCATAGATCTCCAGGAGCTGGGGAAGT
ATGAGCAGTACATTAAGTGGCCTTGGTACATCTGGTTGGGGTTTATAGCAGGGCTGATCGCGATCGTG
ATGGTCACGATCATGCTCTGCTGTATGACGAGCTGCTGCAGCTGCCTCAAGGGCTGCTGCTCTTGTGG
CAGCTGCTGCAAGTTCGACGAGGATGATTCCGAGCCCGTCCTTAAAGGAGTCAAGCTCCACTACACG
DNA corresponding to SEQ ID NO: 19 (JN.1 S containing 12
stabilization mutations except for Q173S and S256Q)
(5′ → 3′) (SEQ ID NO: 19)
ATGTTTGTGTTCCTCGTATTACTTCCGCTGGTGTCGAGCCAGTGCGTGATGCCGTTGTTCAATCTCAT
TACTACTACTCAGGAGTATACGAACTCGTTCACCCGGGGGGTGTATTACCCGGATAAGGTATTCAGGA
GCAGCGTCCTTCACATGACGACGGACCTGTTCCTGCCGTTCTTCTCCAACGTGACGTGGTTTCACGCC
ATCAGCGGGAGCAACGGGACGAAGTACTTCGACAATCCCGTGCTCCCGTTCAATGATGGCGTGTACTT
CGCGTCAACGGAGAAGAGCAATATCATCAGGGGCTGGATCTTCGGGACCACGCTGGACTCGAAGACCC
AGTCCCTGTTGATAGTCAATAACGCGACTAACGTGTTCATCAAGGTGTGTTATTTCCAGTTTTGTAAC
GACCCCTTTCTCGGAGTCTATCACAAGAACAACAAGTCTTGGATGGAGTCCGAGAGCGGGGTCTACGA
TTCTGCGAATAACTGCACCTTTGAATACGTTAGTCAGCCGTTTTTGATGGACCTGGAGGGCAAGCAGG
GGAACTTCAAGAACCTGCGTGAGTTTGTGTTCAAGAACATCGATGGGTACTICAAGATCTACTCGAAG
CACACGCCGATAATCACGCGGGACTTCCCGCAGGGATTTTCGGCGCTGGAGCCTCTGGTAGATCTGCC
CATCGGCATAAACATCACGCGGTTCCAGACCCTGCTTGCCCTCAACAGGTCCTACCTTACTCCGGGTG
ATTCATCCTCCGGGTGGACGGTCGGGGCTGCAGATTACTACGTTGGGTATCTGCAGCCCCGAACGTTT
CTCCTGAAGTATAATGAGAACGGCACAATCACCGACGCGGTTGATTGTGCCCTCGACCCGCTTAGTGA
GACCAAGTGCACACTCAAGTCATTTACGGTGGAGAAGGGGATCTATCAGACCTCCAACTTCCGCGTCC
AGCCGACTGACAGTATCGTTCGGTTCCCGAACGTTACTAATCTGTGTCCGTTCCACGAGGTATTCAAC
GCTACGCGGTTCGCGAGCGTGTACGCGTGGAACCGGACACGGATTAGTAACTGTGTAGCAGACTACAG
TGTGCTATACAACTTCGCCCCGTTCTTCGCGTTCAAGTGTTACGGGGTGTCGCCCACGAAGTTGAACG
ACCTCTGCTTCACCAACGTGTACGCCGACAGCTTTGTGATTAAGGGCAACGAGGTCTCGCAGATCGCC
CCAGGGCAGACGGGGAACATTGCTGACTACAACTACAAGCTGCCTGACGATTTTACCGGTTGCGTTAT
TGCCTGGAATAGCAATAAGCTTGACAGCAAGCATTCCGGAAACTACGATTACTGGTATCGTAGTTTCC
GGAAGAGCAAGCTGAAGCCCTTCGAGAGGGATATCTCTACGGAGATATACCAGGCCGGAAACAAGCCC
TGCAAGGGGAAGGGGCCGAACTGCTACTTCCCCTTGCAGAGCTACGGTTTCCGGCCTACCTACGGGGT
AGGGCACCAGCCCTACCGCGTAGTGGTGCTCTCCTTCGAGCTGCTCCACGCACCGGCGACGGTGTGTG
GGCCGAAGAAGAGCACCAATCTCGTGAAGAACAAATGCGTCAACTTCAATTTCAATGGACTGACTGGA
ACGGGAGTGCTCACGAAGAGCAACAAGAAGTTCCTCCCGTTCCAGCAGTTCGGGCGTGATATCGTTGA
CACCACCGATGCTGTGCGTGATCCCCAGACTCTGGAGATTCTGGATATCACGCCGTGCAGCTTCGGTG
GTGTCAGCGTTATCACGCCCGGAACGAATACATCCAACCAGGTGGCGGTGCTGTATCAGGGCGTGAAT
TGCACCGAGGTATCGGTTGCAATTCACGCCGATCAGCTGACCCCCACCTGGCGGGTGTATTCCACGGG
CAGCAATGTCTTCCAGACACGCGCCGGCTGCTTGATAGGGGCAGAGTATGTGAATAATAGCTACGAGT
GCGACATTCCGATCGGCGCCGGGATTTGTGCGTCGTACCAGACGCAGACCAAGTCCCGGCGCCGAGCT
CGGAGTGTCGCCTCGCAGTCTATTATTGCATATACTATGTCCCTGGGAGCCGAGAACTCAGTAGCATA
TTCGAACAACTCCATTGCTATCCCCACAAATTTTACAATTAGTGTAACCACCGAGATCTTGCCGGTCT
CGATGACCAAGACCTCGGTGGATTGCACTATGTATATTTGTGGGGATAGCACGGAGTGTTCGAATTTG
CTGCTGCAGTACGGCTCCTTCTGCACTCAGCTGAAGCGAGCACTGACTGGGATTGCTGTCGAGCAGGA
CAAGAATACCCAGGAGGTGTTCGCTCAGGTGAAGCAGATCTACAAGACGCCCCCGATCAAGTACTTCG
GGGGCTTCAACTTCAGCCAGATTCTGCCAGACCCATCTAAGCCGAGCAAGAGGTCCTTTATTGAGGAC
CTCTTGTTCAACAAGGTGACTCTGGCAGATGCTGGCTTCATCAAGCAGTACGGCGACTGTCTGGGAGA
CATTGCTGCCCGTGACCTCATCTGCGCGCAGAAGTTCAACGGTCTGACAGTGCTGCCCCCGCTCCTCA
CCGACGAGATGATCGCCCAGTACACCAGCGCGCTGCTGGCTGGGACGATCACCTCGGGGTGGACCTTC
GGAGCGGGGGCAGCACTGCAGATCCCTTTTGCGATGCAGATGGCGTATCGGTTCAATGGAATTGGAGT
GACGCAGAATGTTCTTTACGAGAACCAGAAGCTTATTGCTAACCAGTTCAATAGCGCAATCGGTAAGA
TCCAGGACAGCTTGTTCAGCACCGCGTCTGCCCTGGGGAAGCTGCAAGACGTCGTTAATCACAATGCT
CAGGCGTTGAACACGTTGGTGAAGCAGTTGTCGTCCAAGTTCGGGGCGATCAGTTCGGTGCTGAACGA
TATTCTCAGTCGGCTGGACAAGGTGGAAGCGGAGGTCCAGATAGATCGCCTTATCACCGGTCGCCTCC
AGAGTTTGCAGACGTACGTAACTCAGCAGCTCATCCGAGCTGCTGAGATACGTGCGTCTGCAAACCTG
GCGGCGACCAAGATGAGTGAGTGTGTACTTGGTCAGTCTAAGCGGGTCGACTTCTGCGGGAAGGGCTA
TCACCTGATGTCCTTCCCGCAGAGCGCCCCCCACGGGGTGGTCTTCCTGCACGTGACATATGTGCCGG
CGCAGGAGAAGAACTTCACGACTGCGCCGGCCATATGTCACGACGGGAAGGCCCACTTCCCCCGTGAG
GGGGTGTTCGTGAGCAACGGCACGCACTGGTTCGTCACCCAGCGGAACTTTTACGAGCCACAGATAAT
TACCACTGACAATACCTTTGTCAGTGGTAACTGCGACGTGGTCATAGGCATTGTGAACAACACTGTCT
ATGACCCGTTGCAGTTGGAGCTTGACTCCTTTAAGGAGGAGCTCGACAAGTACTTCAAGAATCATACC
TCGCCGGACGTTGATCTCGGAGACATCTCCGGGATCAACGCTTCGGTGGTCAATATCCAGAAGGAGAT
TGACCGCCTCAACGAGGTGGCCAAGAACCTTAATGAATCGCTCATAGATCTCCAGGAGCTGGGGAAGT
ATGAGCAGTACATTAAGTGGCCTTGGTACATCTGGTTGGGGTTTATAGCAGGGCTGATCGCGATCGTG
ATGGTCACGATCATGCTCTGCTGTATGACGAGCTGCTGCAGCTGCCTCAAGGGCTGCTGCTCTTGTGG
CAGCTGCTGCAAGTTCGACGAGGATGATTCCGAGCCCGTCCTTAAAGGAGTCAAGCTCCACTACACG

TABLE 10
Protein sequences of JN.1 variant spike mutants

Protein corresponding to SEQ ID NO: 14 (JN.1 wild-type spike (JN.1
S)) (SEQ ID NO: 20)
MFVFLVLLPLVSSQCVMPLFNLITTTQSYTNSFTRGVYYPDKVFRSSVLHLTQDLFLPFFSNVTWFHA
ISGTNGTKRFDNPVLPFNDGVYFASTEKSNIIRGWIFGTTLDSKTQSLLIVNNATNVFIKVCEFQFCN
DPFLGVYHKNNKSWMESESGVYSSANNCTFEYVSQPFLMDLEGKQGNFKNLREFVFKNIDGYFKIYSK
HTPIIGRDFPQGFSALEPLVDLPIGINITRFQTLLALNRSYLTPGDSSSGWTAGAADYYVGYLQPRTF
LLKYNENGTITDAVDCALDPLSETKCTLKSFTVEKGIYQTSNFRVQPTESIVRFPNVTNLCPFHEVEN
ATRFASVYAWNRTRISNCVADYSVLYNFAPFFAFKCYGVSPTKLNDLCFTNVYADSFVIKGNEVSQIA
PGQTGNIADYNYKLPDDFTGCVIAWNSNKLDSKHSGNYDYWYRSFRKSKLKPFERDISTEIYQAGNKP
CKGKGPNCYFPLQSYGFRPTYGVGHQPYRVVVLSFELLHAPATVCGPKKSTNLVKNKCVNFNFNGLTG
TGVLTKSNKKFLPFQQFGRDIVDTTDAVRDPQTLEILDITPCSFGGVSVITPGTNTSNQVAVLYQGVN
CTEVSVAIHADQLTPTWRVYSTGSNVFQTRAGCLIGAEYVNNSYECDIPIGAGICASYQTQTKSRRRA
RSVASQSIIAYTMSLGAENSVAYSNNSIAIPTNFTISVTTEILPVSMTKTSVDCTMYICGDSTECSNL
LLQYGSFCTQLKRALTGIAVEQDKNTQEVFAQVKQTYKTPPIKYFGGFNFSQILPDPSKPSKRSFIED
LLFNKVTLADAGFIKQYGDCLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDSLFSTASALGKLQDVVNHNA
QALNTLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQLELDSFKEELDKYFKNHT
SPDVDLGDISGINASVVNIQKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
Protein corresponding to SEQ ID NO: 15 (JN.1 S + Q173S mutation)
(SEQ ID NO: 21)
MFVFLVLLPLVSSQCVMPLFNLITTTQSYTNSFTRGVYYPDKVFRSSVLHLTQDLFLPFFSNVTWFHA
ISGTNGTKRFDNPVLPFNDGVYFASTEKSNIIRGWIFGTTLDSKTQSLLIVNNATNVFIKVCEFQFCN
DPFLGVYHKNNKSWMESESGVYSSANNCTFEYVSSPFLMDLEGKQGNFKNLREFVFKNIDGYFKIYSK
HTPIIGRDFPQGFSALEPLVDLPIGINITRFQTLLALNRSYLTPGDSSSGWTAGAADYYVGYLQPRTF
LLKYNENGTITDAVDCALDPLSETKCTLKSFTVEKGIYQTSNFRVQPTESIVRFPNVINLCPFHEVEN
ATRFASVYAWNRTRISNCVADYSVLYNFAPFFAFKCYGVSPTKLNDLCFTNVYADSFVIKGNEVSQIA
PGQTGNIADYNYKLPDDFTGCVIAWNSNKLDSKHSGNYDYWYRSFRKSKLKPFERDISTEIYQAGNKP
CKGKGPNCYFPLQSYGFRPTYGVGHQPYRVVVLSFELLHAPATVCGPKKSTNLVKNKCVNFNFNGLTG
TGVLTKSNKKFLPFQQFGRDIVDTTDAVRDPQTLEILDITPCSFGGVSVITPGTNTSNQVAVLYQGVN
CTEVSVAIHADQLTPTWRVYSTGSNVFQTRAGCLIGAEYVNNSYECDIPIGAGICASYQTQTKSRRRA
RSVASQSIIAYTMSLGAENSVAYSNNSIAIPTNFTISVTTEILPVSMTKTSVDCTMYICGDSTECSNL
LLQYGSFCTQLKRALTGIAVEQDKNTQEVFAQVKQIYKTPPIKYFGGFNFSQILPDPSKPSKRSFIED
LLFNKVTLADAGFIKQYGDCLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDSLFSTASALGKLQDVVNHNA
QALNTLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQLELDSFKEELDKYFKNHT
SPDVDLGDISGINASVVNIQKEIDRINEVAKNLNESLIDLQELGKYEQYIKWPWYIWLGFIAGLIAIV
MVTIMLCCMTSCCSCLKGCCSCGSCCKFDEDDSEPVLKGVKLHYT
Protein corresponding to SEQ ID NO: 16 (JN.1 S + S256Q mutation)
(SEQ ID NO: 22)
MFVFLVLLPLVSSQCVMPLFNLITTTQSYTNSFTRGVYYPDKVFRSSVLHLTQDLFLPFFSNVTWFHA
ISGTNGTKRFDNPVLPFNDGVYFASTEKSNIIRGWIFGTTLDSKTQSLLIVNNATNVFIKVCEFQFCN
DPFLGVYHKNNKSWMESESGVYSSANNCTFEYVSQPFLMDLEGKQGNFKNLREFVFKNIDGYFKIYSK
HTPIIGRDFPQGFSALEPLVDLPIGINITRFQTLLALNRSYLTPGDSSQGWTAGAADYYVGYLQPRTF
LLKYNENGTITDAVDCALDPLSETKCTLKSFTVEKGIYQTSNFRVQPTESIVRFPNVTNLCPFHEVEN
ATRFASVYAWNRTRISNCVADYSVLYNFAPFFAFKCYGVSPTKLNDLCFTNVYADSFVIKGNEVSQIA
PGQTGNIADYNYKLPDDFTGCVIAWNSNKLDSKHSGNYDYWYRSFRKSKLKPFERDISTEIYQAGNKP
CKGKGPNCYFPLQSYGFRPTYGVGHQPYRVVVLSFELLHAPATVCGPKKSTNLVKNKCVNFNFNGLTG
TGVLTKSNKKFLPFQQFGRDIVDTTDAVRDPQTLEILDITPCSFGGVSVITPGTNTSNQVAVLYQGVN
CTEVSVAIHADQLTPTWRVYSTGSNVFQTRAGCLIGAEYVNNSYECDIPIGAGICASYQTQTKSRRRA
RSVASQSIIAYTMSLGAENSVAYSNNSIAIPTNFTISVTTEILPVSMTKTSVDCTMYICGDSTECSNL
LLQYGSFCTQLKRALTGIAVEQDKNTQEVFAQVKQTYKTPPIKYFGGFNFSQILPDPSKPSKRSFIED
LLFNKVTLADAGFIKQYGDCLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDSLFSTASALGKLQDVVNHNA
QALNTLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQLELDSFKEELDKYFKNHT
SPDVDLGDISGINASVVNIQKEIDRINEVAKNLNESLIDLQELGKYEQYIKWPWYIWLGFIAGLIAIV
MVTIMLCCMTSCCSCLKGCCSCGSCCKFDEDDSEPVLKGVKLHYT
Protein corresponding to SEQ ID NO: 17 (JN.1 S + Q173S and S256Q
double mutations) (SEQ ID NO: 23)
MFVFLVLLPLVSSQCVMPLFNLITTTQSYTNSFTRGVYYPDKVFRSSVLHLTQDLFLPFFSNVTWFHA
ISGTNGTKRFDNPVLPFNDGVYFASTEKSNIIRGWIFGTTLDSKTQSLLIVNNATNVFIKVCEFQFCN
DPFLGVYHKNNKSWMESESGVYSSANNCTFEYVSSPFLMDLEGKQGNFKNLREFVFKNIDGYFKIYSK
HTPIIGRDFPQGFSALEPLVDLPIGINITRFQTLLALNRSYLTPGDSSQGWTAGAADYYVGYLQPRTF
LLKYNENGTITDAVDCALDPLSETKCTLKSFTVEKGIYQTSNFRVQPTESIVRFPNVINLCPFHEVFN
ATRFASVYAWNRTRISNCVADYSVLYNFAPFFAFKCYGVSPTKLNDLCFTNVYADSFVIKGNEVSQIA
PGQTGNIADYNYKLPDDFTGCVIAWNSNKLDSKHSGNYDYWYRSFRKSKLKPFERDISTEIYQAGNKP
CKGKGPNCYFPLQSYGFRPTYGVGHQPYRVVVLSFELLHAPATVCGPKKSTNLVKNKCVNFNFNGLTG
TGVLTKSNKKFLPFQQFGRDIVDTTDAVRDPQTLEILDITPCSFGGVSVITPGTNTSNQVAVLYQGVN
CTEVSVAIHADQLTPTWRVYSTGSNVFQTRAGCLIGAEYVNNSYECDIPIGAGICASYQTQTKSRRRA
RSVASQSIIAYTMSLGAENSVAYSNNSIAIPTNFTISVTTEILPVSMTKTSVDCTMYICGDSTECSNL
LLQYGSFCTQLKRALTGIAVEQDKNTQEVFAQVKQTYKTPPIKYFGGFNFSQILPDPSKPSKRSFIED
LLFNKVTLADAGFIKQYGDCLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDSLFSTASALGKLQDVVNHNA
QALNTLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQLELDSFKEELDKYFKNHT
SPDVDLGDISGINASVVNIQKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
Protein corresponding to SEQ ID NO: 18 (JN.1 S containing all 12
stabilization mutations) (SEQ ID NO: 24)
MFVFLVLLPLVSSQCVMPLFNLITTTQEYTNSFTRGVYYPDKVFRSSVLHMTTDLFLPFFSNVTWFHA
ISGSNGTKYFDNPVLPFNDGVYFASTEKSNIIRGWIFGTTLDSKTQSLLIVNNATNVFIKVCYFQFCN
DPFLGVYHKNNKSWMESESGVYDSANNCTFEYVSSPFLMDLEGKQGNFKNLREFVFKNIDGYFKIYSK
HTPIITRDFPQGFSALEPLVDLPIGINITRFQTLLALNRSYLTPGDSSQGWTVGAADYYVGYLQPRTF
LLKYNENGTITDAVDCALDPLSETKCTLKSFTVEKGIYQTSNFRVQPTDSIVRFPNVTNLCPFHEVEN
ATRFASVYAWNRTRISNCVADYSVLYNFAPFFAFKCYGVSPTKLNDLCFTNVYADSFVIKGNEVSQIA
PGQTGNIADYNYKLPDDFTGCVIAWNSNKLDSKHSGNYDYWYRSFRKSKLKPFERDISTEIYQAGNKP
CKGKGPNCYFPLQSYGFRPTYGVGHQPYRVVVLSFELLHAPATVCGPKKSTNLVKNKCVNFNFNGLTG
TGVLTKSNKKFLPFQQFGRDIVDTTDAVRDPQTLEILDITPCSFGGVSVITPGTNTSNQVAVLYQGVN
CTEVSVAIHADQLTPTWRVYSTGSNVFQTRAGCLIGAEYVNNSYECDIPIGAGICASYQTQTKSRRRA
RSVASQSIIAYTMSLGAENSVAYSNNSIAIPTNFTISVTTEILPVSMTKTSVDCTMYICGDSTECSNL
LLQYGSFCTQLKRALTGIAVEQDKNTQEVFAQVKQIYKTPPIKYFGGFNFSQILPDPSKPSKRSFIED
LLFNKVTLADAGFIKQYGDCLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDSLFSTASALGKLQDVVNHNA
QALNTLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQLELDSFKEELDKYFKNHT
SPDVDLGDISGINASVVNIQKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
Protein corresponding to SEQ ID NO: 19 (JN.1 S containing 12
stabilization mutations except for Q173S and S256Q) (SEQ ID NO: 25)
MFVFLVLLPLVSSQCVMPLFNLITTTQEYTNSFTRGVYYPDKVFRSSVLHMTTDLFLPFFSNVTWFHA
ISGSNGTKYFDNPVLPFNDGVYFASTEKSNIIRGWIFGTTLDSKTQSLLIVNNATNVFIKVCYFQFCN
DPFLGVYHKNNKSWMESESGVYDSANNCTFEYVSQPFLMDLEGKQGNFKNLREFVFKNIDGYFKIYSK
HTPIITRDFPQGFSALEPLVDLPIGINITRFQTLLALNRSYLTPGDSSSGWTVGAADYYVGYLQPRTF
LLKYNENGTITDAVDCALDPLSETKCTLKSFTVEKGIYQTSNFRVQPTDSIVRFPNVTNLCPFHEVEN
ATRFASVYAWNRTRISNCVADYSVLYNFAPFFAFKCYGVSPTKLNDLCFTNVYADSFVIKGNEVSQIA
PGQTGNIADYNYKLPDDFTGCVIAWNSNKLDSKHSGNYDYWYRSFRKSKLKPFERDISTEIYQAGNKP
CKGKGPNCYFPLQSYGFRPTYGVGHQPYRVVVLSFELLHAPATVCGPKKSTNLVKNKCVNFNFNGLTG
TGVLTKSNKKFLPFQQFGRDIVDTTDAVRDPQTLEILDITPCSFGGVSVITPGTNTSNQVAVLYQGVN
CTEVSVAIHADQLTPTWRVYSTGSNVFQTRAGCLIGAEYVNNSYECDIPIGAGICASYQTQTKSRRRA
RSVASQSIIAYTMSLGAENSVAYSNNSIAIPTNFTISVTTEILPVSMTKTSVDCTMYICGDSTECSNL
LLQYGSFCTQLKRALTGIAVEQDKNTQEVFAQVKQIYKTPPIKYFGGFNFSQILPDPSKPSKRSFIED
LLFNKVTLADAGFIKQYGDCLGDIAARDLICAQKFNGLTVLPPLLTDEMIAQYTSALLAGTITSGWTF
GAGAALQIPFAMQMAYRFNGIGVTQNVLYENQKLIANQFNSAIGKIQDSLFSTASALGKLQDVVNHNA
QALNTLVKQLSSKFGAISSVLNDILSRLDKVEAEVQIDRLITGRLQSLQTYVTQQLIRAAEIRASANL
AATKMSECVLGQSKRVDFCGKGYHLMSFPQSAPHGVVFLHVTYVPAQEKNFTTAPAICHDGKAHFPRE
GVFVSNGTHWFVTQRNFYEPQIITTDNTFVSGNCDVVIGIVNNTVYDPLQLELDSFKEELDKYFKNHT
SPDVDLGDISGINASVVNIQKEIDRINEVAKNLNESLIDLQELGKYEQYIKWPWYIWLGFIAGLIAIV
MVTIMLCCMTSCCSCLKGCCSCGSCCKFDEDDSEPVLKGVKLHYT

### Example 6-2. Expression of JN.1 Variant Spike Mutant Proteins

The above-described spike mutant constructs were transfected into HEK293T cell lines. Protein expression was evaluated 24 hours post-transfection by harvesting the cells and performing Western blot analysis. β-actin was used as an internal control to normalize the protein expression levels.

### Example 6-3. Verification of the Stabilizing Effect of JN.1 Variant Spike Mutant Proteins

As a result, spike mutant variants containing Q173S and S256Q substitutions exhibited increased spike protein expression compared to the JN.1 wild-type. Notably, the double mutant containing both Q173S and S2560 substitutions showed the most pronounced increase in expression levels.

In contrast, the mutant in which Q173S and S256Q were removed from the 12 stabilization mutations exhibited a marked reduction in expression levels (see FIG. 11). These findings demonstrate that the Q173 and S256 residues represent universal stabilization sites that enhance the expression and structural stability of spike proteins, thereby validating the stabilization design strategy of the present invention.

This specification omits detailed descriptions of content that can be readily understood and inferred by those skilled in the art. Various modifications may be made without departing from the technical spirit or essential features of the present invention, in addition to the specific examples described herein. Accordingly, the present invention may be implemented in ways other than those specifically described and exemplified in this specification, as would be understood by those skilled in the art.

1. A polypeptide for preventing or treating SARS-CoV-2 infection, the polypeptide comprising one or more substitutions selected from a group consisting of:
substitution of an amino acid corresponding to position 27 with E;
substitution of an amino acid corresponding to position 50 with M;
substitution of an amino acid corresponding to position 52 with T;
substitution of an amino acid corresponding to position 73 with S;
substitution of an amino acid corresponding to position 78 with Y;
substitution of an amino acid corresponding to position 132 with Y;
substitution of an amino acid corresponding to position 161 with D;
substitution of an amino acid corresponding to position 173 with S or K;
substitution of an amino acid corresponding to position 213 with T;
substitution of an amino acid corresponding to position 256 with Q or N;
substitution of an amino acid corresponding to position 260 with V; and
substitution of an amino acid corresponding to position 324 with D,
provided that the position numberings are based on the amino acid positions within a reference amino acid sequence of SEQ ID NO: 1.

2. The polypeptide for preventing or treating SARS-CoV-2 infection as set forth in claim 1, wherein the SARS-CoV-2 comprises variants of SARS-CoV-2.

3. The polypeptide for preventing or treating SARS-CoV-2 infection as set forth in claim 2, wherein the variants of SARS-CoV-2 are one or more selected from a group consisting of Alpha, Beta, Gamma, Delta, and Omicron variants.

4. The polypeptide for preventing or treating SARS-CoV-2 infection as set forth in claim 1, wherein the amino acid substitution is substitution of an amino acid corresponding to position 173 with S or K; and substitution of an amino acid corresponding to position 256 with Q or N.

5. The polypeptide for preventing or treating SARS-CoV-2 infection as set forth in claim 1, wherein the amino acid substitution is
substitution of an amino acid corresponding to position 52 with T;
substitution of an amino acid corresponding to position 161 with D;
substitution of an amino acid corresponding to position 173 with S;
substitution of an amino acid corresponding to position 256 with Q; and
substitution of an amino acid corresponding to position 324 with D.

6. The polypeptide for preventing or treating SARS-CoV-2 infection as set forth in claim 5, wherein the polypeptide comprises an amino acid sequence of SEQ ID NO: 2.

7. The polypeptide for preventing or treating SARS-CoV-2 infection according to claim 4, wherein the substitutions at positions 173 and 256 provide universal stabilization sites that enhance the expression and structural stability of the spike protein.

8. The polypeptide for preventing or treating SARS-CoV-2 infection according to claim 7, wherein the polypeptide comprises Q173S and S256Q double mutations, and the mutations function as universal stabilization sites that enhance the expression and structural stability of the spike protein.

9. The polypeptide for preventing or treating SARS-CoV-2 infection according to claim 7, wherein the polypeptide is based on a JN.1 variant spike protein and comprises Q173 and S256 mutations.

10. The polypeptide for preventing or treating SARS-CoV-2 infection as set forth in claim 1, wherein the amino acid substitution is
substitution of an amino acid corresponding to position 27 with E;
substitution of an amino acid corresponding to position 50 with M;
substitution of an amino acid corresponding to position 73 with S;
substitution of an amino acid corresponding to position 78 with Y;
substitution of an amino acid corresponding to position 132 with Y;
substitution of an amino acid corresponding to position 173 with K;
substitution of an amino acid corresponding to position 213 with T;
substitution of an amino acid corresponding to position 256 with N; and
substitution of an amino acid corresponding to position 260 with V.

11. The polypeptide for preventing or treating SARS-CoV-2 infection as set forth in claim 10, wherein the polypeptide comprises an amino acid sequence of SEQ ID NO: 3.

12. A polypeptide for preventing or treating SARS-CoV-2 infection, the polypeptide comprising amino acid sequence of SEQ ID NO: 4 or SEQ ID NO: 5.

13. The polypeptide for preventing or treating SARS-CoV-2 infection as set forth in claim 10, wherein the SARS-CoV-2 comprises variants of SARS-CoV-2.

14. The polypeptide for preventing or treating SARS-CoV-2 infection as set forth in claim 13, wherein the variants of SARS-CoV-2 are one or more selected from a group consisting of Alpha, Beta, Gamma, Delta, and Omicron variants.

15. A vaccine composition for preventing SARS-CoV-2 infection, the vaccine composition comprising the polypeptide of claim 1 as an active ingredient.

16. (canceled)

17. A polynucleotide for preventing or treating SARS-CoV-2 infection, the polynucleotide encoding the polypeptide of claim 1.

18. A vaccine composition for preventing SARS-CoV-2 infection, the vaccine composition comprising the polynucleotide of claim 17 as an active ingredient.

19. (canceled)

20. The polypeptide for preventing or treating SARS-CoV-2 infection according to claim 5, wherein the substitutions at positions 173 and 256 provide universal stabilization sites that enhance the expression and structural stability of the spike protein.

21. The polypeptide for preventing or treating SARS-CoV-2 infection according to claim 6, wherein the substitutions at positions 173 and 256 provide universal stabilization sites that enhance the expression and structural stability of the spike protein.


SEQUENCE LISTING (25 sequences)

SEQ ID NO: 1 - protein, 685 aa, SARS-CoV-2 Wuhan-Hu-1 strain
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 2 - protein, 679 aa, SARS-CoV-2 S
MFVFLVLLPLVSSQCVNLTTRTQLPPAYTNSFTRGVYYPDKVFRSSVLHSTTDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYDSANNCTFEYVSSPFLMDLEGKQGNFKNLREFVFKNIDGYFKIYSKHTPINLVRDLPQGFSALEPLVDLPIGINITRFQTLLALHRSYLTPGDSSQGWTAGAAAYYVGYLQPRTFLLKYNENGTITDAVDCALDPLSETKCTLKSFTVEKGIYQTSNFRVQPTDSIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPA

SEQ ID NO: 3 - protein, 679 aa, SARS-CoV-2 recombinant S
MFVFLVLLPLVSSQCVNLITRTQEYTNSFTRGVYYPDKVFRSSVLHMTQDLFLPFFSNVTWFHAIHVSGSNGTKYFDNPVLPFNDGVYFASTEKSNIIRGWIFGTTLDSKTQSLLIVNNATNVVIKVCYFQFCNDPFLDVYYHKNNKSWMESEFRVYSSANNCTFEYVSKPFLMDLEGKQGNFKNLREFVFKNIDGYFKIYSKHTPINLTRDLPQGFSALEPLVDLPIGINITRFQTLLALHRSYLTPGDSSNGWTVGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHA

SEQ ID NO: 4 - protein, 679 aa, SARS-CoV-2 recombinant S
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPA

SEQ ID NO: 5 - protein, 679 aa, SARS-CoV-2 recombinant S
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHA

SEQ ID NO: 6 - RNA, 2037 nt, SARS-CoV-2 recombinant S
  source 1..2037: mol_type = mRNA
atgttcgtgttccttgtgctgctgcctctggtcagctctcagtgcgtaaacctgacaacccggactcagctgcctcctgcctacaccaactctttcaccagaggcgtgtactaccctgacaaggtgttcagaagcagcgtgctgcacagcacacaggatctgttcctgccatttttcagcaacgtgacctggttccacgccataagcggcaccaacggcacgaagcggtttgataaccccgtcctccctttcaacgacggcgtatacttcgccagcaccgagaagagcaacatcatccggggatggatcttcggtacaacgctggacagcaagacccagagcctgctgatcgtgaacaatgccaccaacgtggtgattaaggtgtgcgagttccagttctgcaacgacccttttctgggcgtgtaccacaagaacaacaagagctggatggaaagcgagttcagagtgtacagcagcgcaaacaattgtacctttgagtacgtgtctcagcctttcctgatggacctggagggcaaacagggcaatttcaaaaacctgagagagttcgtgttcaagaacatcgacggatacttcaagatctacagcaagcacacaccgatcaatctggtgcgcgatctgccccagggattttccgctctggaacccctggtggacctccctatcggcatcaatataacccggtttcagacgctgctggccctgcacagaagttacctgacccctggcgattcctccagcggctggaccgccggtgccgctgcctactacgtgggctacctgcagccccgcaccttcctgctgaagtacaatgaaaacggaaccatcactgatgccgtggactgcgccctggaccctctgtccgagacaaagtgtacactgaagagcttcacagtggagaagggcatctatcagacctcgaattttagagtgcagccaacagaaagcatcgtgagatttcccaacatcaccaacctgtgcccttttggcgaggtcttcaacgccacaagattcgcctctgtatatgcctggaacagaaagagaatctctaactgcgtggcagactactcggttctgtacaactcagcctctttttccacattcaagtgctacggcgtgtctcctacgaagctgaacgacctgtgcttcacaaacgtgtacgccgacagcttcgtgatcagaggagatgaggtgagacagatcgccccaggccagacaggcaagatcgccgactacaattataagctgcccgatgatttcacagggtgcgtgatcgcctggaactcaaataacctcgatagcaaggtgggcggaaattacaactaccgctacagactgtttcggaagagtaaccttaaacctttcgagagagacatctccaccgagatctaccaggccggatctaagccttgcaatggcgtggaaggattcaactgctacttccctctgcagtcttacggctttcagcctacaaatggagtgggctaccagccctaccgtgtggtggtcctgagctttgagctgctgcatgctcctgccaccgtctgcggccctaagaagtctaccaacctggtgaagaacaaatgcgtgaacttcaatttcaacgggctgaccggcacaggcgtgctgaccgaatctaacaagaaattcctgcccttccagcagttcggccgggacattgccgacaccacagacgccgtgcgggatccccagactctggaaatcctggacatcaccccttgcagcttcggcggcgtgagcgtgattaccccaggcaccaacacatctaatcaggttgccgtgctgtaccagggcgttaactgcacagaagtgcctgtcgccatccacgccgaccagctgacccctacgtggcgggtttacagcaccggcagcaacgtgttccagactagagccggctgcctgatcggcgccgagcacgtcaacaatagctacgagtgtgacattcctatcggcgccggcatctgcgcctcttaccagacccagaccaacagcccagca

SEQ ID NO: 7 - RNA, 2037 nt, SARS-CoV-2 recombinant S
  source 1..2037: mol_type = mRNA
atgttcgtgttcctggtgctgctgcctctggtgagcagccagtgcgtgaatctgattacaagaactcaggagtacactaactctttcaccagaggagtgtactaccccgataaggtgtttaggtcaagcgtgctgcacatgactcaggacctgttcctgcctttcttttctaatgtgacctggttccacgcaatccacgtgagcggcagcaacggcacgaaatatttcgataatcctgtgctgcccttcaacgacggcgtgtacttcgcctccacagagaagtcaaacattattcgggggtggatcttcggcaccacactggacagtaaaacccagtccctgctgatcgtgaacaatgctaccaacgtggtgattaaggtgtgctactttcagttctgcaatgatccattcctggatgtgtactaccacaagaacaataagtcctggatggaaagcgaattcagagtgtattcctctgcaaataactgtacattcgagtacgtgtccaagccattcctcatggaccttgaaggcaagcaggggaattttaaaaacctgcgggagtttgtgtttaagaatatcgatggctacttcaagatttattccaaacacactcccatcaacctgaccagggacctgccccagggcttttccgccctggagcccctggtggacctgcccattggcattaacattacaagattccagaccctgctggctctgcacaggtcctatctgacacctggcgatagcagcaatggctggaccgtcggggcagccgcctattacgtgggatacctgcagccccggacctttctgctgaagtacaatgagaacggcacaatcacagacgctgtggactgcgccctggacccactgagcgagactaagtgcactctgaaatcattcactgtggagaaggggatttatcagacatctaacttcagagtccagcctaccgagagtatcgtgaggttccccaacatcaccaacctgtgcccttttcacgaggtgtttaacgccaccaccttcgcctcagtgtatgcctggaacaggaagagaatcagcaattgcgtggcagactactccgtgctgtacaacttcgcacccttcttcgcctttaagtgttacggcgtgagccccaccaaactgaacgatctgtgcttcacaaatgtgtacgccgatagttttgtgatccgggggaatgaggtgtctcaaatcgcccccggccagaccggaaacatcgctgattataactacaagctgcccgatgattttaccgggtgcgtgatcgcctggaatagtaataagctggatagcaaagtgtctggcaactataactatctgtaccggctgttcaggaaatccaagctgaagcccttcgagcgcgacatctctaccgaaatttaccaggccggtaacaagccatgtaacggcgtggctggctttaattgctacttcccactgcagagctacgggttccggcccacctatggggtggggcatcagccctaccgcgtggtggtgctgtccttcgagctgctgcacgccccagctaccgtgtgcgggcccaagaagtcaactaatctggtcaagaacaagtgtgtcaacttcaacttcaatggcctgactgggacaggcgtgctcaccgagagcaacaagaaattcctgccttttcagcagttcggcagggacatcgccgacaccacagatgccgttcgggacccacagaccctggaaatcctggatattactccatgcagcttcggcggcgtgtccgtgattacacctggaaccaacaccagcaaccaggtggccgtgctgtaccagggcgtgaactgcaccgaggtgcctgtggcaatacacgccgaccagctgactcccacatggcgagtgtattcaaccggctccaatgtgtttcagactagagccggctgcctgatcggagctgagcacgtgaataactcttatgagtgcgatatccccatcggagccggcatctgcgcatcctatcagacacagacaaacagccacgcc

SEQ ID NO: 8 - RNA, 2037 nt, SARS-CoV-2 recombinant S; identical to SEQ ID NO: 6
  source 1..2037: mol_type = mRNA

SEQ ID NO: 9 - RNA, 2037 nt, SARS-CoV-2 recombinant S
  source 1..2037: mol_type = mRNA
atgtttgtgtttctggttctcctccccctggtcagtagccagtgcgtaaatctgatcactcgcacacaatcatacacgaattctttcacacgcggggtctattatcctgataaggtttttagaagttctgtactccactctacacaagacctgttcctgccgtttttctccaacgtaacatggtttcacgcgattcatgtctccggaacgaacgggacaaaacggtttgataatcctgttctcccttttaatgacggggtttatttcgcaagcacagagaagtcaaacattattagggggtggatctttggtaccactttggattccaaaacacaaagcctgcttatagtgaacaatgcgaccaatgtggtaataaaagtctgtgaattccagttctgcaatgatcctttccttgatgtctattaccacaagaacaacaagagctggatggagtccgaattccgggtgtatagcagcgctaacaattgtacgtttgagtacgtctctcaacctttcttgatggacctggagggtaaacaggggaacttcaaaaaccttcgggaattcgttttcaagaatattgacggctacttcaaaatatacagtaaacatactcccattaacctcggacgcgacctcccacaaggcttcagcgcgcttgaaccactcgtagacctccctatcggaatcaatatcacccgcttccaaactctcctggccctccaccggtcatatctgactcccggtgattctagctctggttggacagcaggggcagccgcatactatgtcgggtatcttcaacctcgcactttcctgcttaagtacaacgaaaatggcactattaccgacgctgtagattgcgctcttgacccgttgagtgagacgaagtgcaccctcaagagctttaccgtcgagaaggggatctaccagacgtcaaattttagagtccaaccgacggagtctatcgtccggtttcctaacattactaacctgtgtccatttcatgaagtgttcaatgcaacgaccttcgcatctgtgtatgcttggaatcgcaagaggattagtaattgcgttgctgattactccgttctttacaacttcgccccgttcttcgcttttaagtgctacggtgttagtccaactaaactgaacgacctttgcttcactaatgtttacgcagattcatttgtcattcggggcaatgaagtcagccagatcgcccctggccaaacggggaatatagctgactacaattacaagctgcccgacgactttacggggtgtgtcattgcgtggaacagtaataaactcgactcaaaggttagtggtaactataattatctttatcggttgtttagaaagagtaaattgaagccttttgaaagggatatttcaacagagatatatcaagcgggtaataagccatgcaacggcgtggcgggctttaactgttactttcctctgcaatcctacggctttagaccaacatatggagtcgggcatcaaccctatcgggttgtggttttgtctttcgagctgctccacgcaccggccactgtatgcggccccaaaaaaagtaccaatttggtgaaaaataaatgtgtgaattttaattttaacgggttgacaggaactggggtactcactgaatctaataaaaaattcctcccatttcaacaattcgggagagatatagcagatacaacagatgccgtgcgggacccccagacgttggaaatccttgatatcacgccatgttccttcggcggggtaagcgttataacccccgggacgaacaccagtaatcaagttgcggtcctctatcagggtgttaactgtacggaagttcctgtggccatccacgctgaccaactgacgccaacttggagagtatacagcactggctccaatgtctttcagacacgcgcgggctgtcttattggtgctgagcatgtcaataacagttacgagtgcgatatcccgataggagctggcatatgtgcctcataccaaacccagacgaattctcatgcc

SEQ ID NO: 10 - protein, 1267 aa, SARS-CoV-2 recombinant S
MFVFLVLLPLVSSQCVNLTTRTQLPPAYTNSFTRGVYYPDKVFRSSVLHSTTDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYDSANNCTFEYVSSPFLMDLEGKQGNFKNLREFVFKNIDGYFKIYSKHTPINLVRDLPQGFSALEPLVDLPIGINITRFQTLLALHRSYLTPGDSSQGWTAGAAAYYVGYLQPRTFLLKYNENGTITDAVDCALDPLSETKCTLKSFTVEKGIYQTSNFRVQPTDSIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1267 aa, SARS-CoV-2 recombinant S
MFVFLVLLPLVSSQCVNLITRTQEYTNSFTRGVYYPDKVFRSSVLHMTQDLFLPFFSNVTWFHAIHVSGSNGTKYFDNPVLPFNDGVYFASTEKSNIIRGWIFGTTLDSKTQSLLIVNNATNVVIKVCYFQFCNDPFLDVYYHKNNKSWMESEFRVYSSANNCTFEYVSKPFLMDLEGKQGNFKNLREFVFKNIDGYFKIYSKHTPINLTRDLPQGFSALEPLVDLPIGINITRFQTLLALHRSYLTPGDSSNGWTVGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGFNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - RNA, 3801 nt, SARS-CoV-2 recombinant S
  source 1..3801: mol_type = mRNA
atgttcgtgtttctggtcctgctgcctctggtgtctagccagtgcgtgaacctgaccacccggacccaactgcctcctgcctacaccaatagctttacaagaggcgtgtattaccccgataaggtgttcagaagcagcgtgctgcacagcacaaccgacctgttcctgcctttcttcagcaacgtgacctggttccacgccatcagcggaactaacggcaccaagagattcgacaaccccgtgctgccattcaacgacggcgtttatttcgcctctaccgagaagagcaacatcatcagaggctggatcttcggcaccaccctggacagcaaaacccagagtctgctgatcgtgaataacgccaccaacgtcgtcatcaaggtgtgcgagttccagttctgcaacgaccctttcttgggcgtgtaccacaagaacaacaagtcctggatggaaagcgaatttagagtgtacgactctgccaacaactgcaccttcgagtacgtgtccagccctttcctgatggaccttgaaggaaaacagggcaacttcaagaatctgagggaattcgtgttcaaaaacatcgacggctacttcaagatctacagcaagcacacccctatcaacctggtgcgggacttgccgcaaggcttcagcgccctggaacctctggtggacctgcccatcggcattaacatcacccggttccagaccctgctggccctccacagatcctacctgacccctggcgatagctctcagggctggaccgccggcgccgctgcttactacgtgggatacctgcagcctagaacattcctgctgaagtacaacgagaacggcacaatcaccgacgccgtggactgcgccctggaccccctgtctgagacaaagtgcacactgaaaagcttcaccgtggaaaaaggcatctaccaaaccagcaatttcagagtgcagcctacagattccatcgtgcggtttccaaatatcacaaacctgtgtccttttggcgaggtgttcaacgccaccagattcgcctccgtgtatgcctggaacagaaagagaatcagcaactgcgtggccgactacagcgtgctgtacaacagcgcctcctttagcacattcaagtgctacggcgtcagccctacaaagctgaacgacctgtgcttcaccaacgtctacgccgacagctttgtgatccggggcgacgaggtgcggcagatcgcccctggccagactggcaagatcgctgactacaattataagctgccagatgacttcaccggatgtgtgatcgcctggaactccaacaacctggatagcaaggtgggcggaaattacaactacagataccggctgttccggaagtctaacctgaagccttttgaaagagatattagcactgagatctaccaggccggcagcaagccctgtaatggagtggaaggcttcaactgctactttcctctgcaaagctacgggtttcagcctacaaatggcgtgggctaccagccctacagagtggtggtgctgagcttcgagctgctgcatgctcctgctacagtgtgcggccctaagaagtccacaaatctggtgaagaacaaatgtgtgaacttcaatttcaacggcctgacaggcaccggcgttctgacagagagcaacaagaaattcctgcccttccagcagttcggcagagacatcgcagatacaaccgatgccgtgagagatccccagaccctggagatcctggacatcaccccctgtagcttcggcggcgttagcgtgatcaccccaggcaccaatacctctaaccaggtcgctgtgctgtaccagggagtgaactgcaccgaggtgcctgtggctatccacgccgatcagctgacccccacatggcgggtgtactctacaggaagcaatgtgtttcagacccgcgccggttgtctgatcggcgccgagcacgtgaacaattcatacgagtgcgacatccctatcggagctggcatctgcgcctcctaccagacccagaccaacagccccgcctctgtagcctctcaaagcatcattgcctacacaatgagcctgggcgccgagaactccgttgcttatagtaataatagcattgcaatccctaccaacttcaccatcagcgtgaccacagagattctgcctgtgagcatgaccaagacctccgtggactgtaccatgtacatctgtggcgactcgactgaatgcagcaacctgctgctccaatacggcagcttctgcacacagctgaaccgggctctgaccggcatcgccgtggaacaggacaagaacacccaagaggtgtttgcccaggtgaagcagatttacaagacccctcccatcaaggacttcggcggtttcaactttagccagatcctgcctgatccttctaaacccagcaagcggagccccatcgaggacctgctgttcaacaaggtgaccctggctgacgccggattcatcaagcagtatggcgactgtctgggcgatatcgctgccagagatctcatctgtgcccagaagttcaacggactgactgtgctaccacccttactgaccgatgagatgatcgctcagtacacatctgccctgctggctgggaccatcaccagcggctggacattcggcgccggccccgccctgcaaatcccgtttcctatgcagatggcctatagattcaacggaatcggcgtgacccagaacgtcctgtatgagaaccagaagctgatcgctaatcagttcaacagcgcaatcggcaaaatccaagactctctgagcagcacaccctccgctctgggcaagctgcaggatgtggtgaaccaaaacgcccaagcccttaacaccctggtgaaacagctgagcagcaactttggtgccatcagcagcgtgcttaacgacattctgtctaggctggacccccctgaggccgaagtgcagatcgacagactgatcaccggaagactgcagagcctgcaaacctacgtgacccaacagttgatcagagccgctgagatccgggcgagcgccaacctggccgccacaaagatgagcgaatgtgtgctggggcagagcaaaagagtggacttctgcggcaaaggctatcacctgatgagctttcctcagagcgccccccacggcgtggtgttcctgcacgtgacatacgtgcctgctcaggagaagaacttcaccaccgcccctgccatctgccacgacggcaaggctcacttcccaagagaaggcgtgttcgtcagcaatggcacccactggttcgtgacccaaagaaacttctacgagcctcagatcatcacaaccgacaacacattcgtgagcgggaactgcgacgtggtaatcggcattgtgaacaataccgtgtacgaccctctgcagccagaactggattccttcaaggaagagctggacaagtactttaagaaccacacaagccctgacgtggacctgggcgacataagcggtatcaacgcctcagtggtcaacatccagaaagagatcgacaggttaaacgaagtggccaagaacctgaatgagtccctgatcgatctgcaggagctgggcaagtacgaacagtacatcaagtggccttggtacatctggctgggattcattgccggattgatcgccatcgtcatggtgaccatcatgctgtgctgcatgacgagctgctgctcttgcctgaagggctgctgtagctgtggctcctgttgtaaattcgacgaagatgacagcgagcccgtgctgaagggagtgaagctgcattacacc

SEQ ID NO: 13 - RNA, 3801 nt, SARS-CoV-2 recombinant S
  source 1..3801: mol_type = mRNA
atgttcgtgttcctggtgctgctgcctctggtgagcagccagtgcgtgaatctgattacaagaactcaggagtacactaactctttcaccagaggagtgtactaccccgataaggtgtttaggtcaagcgtgctgcacatgactcaggacctgttcctgcctttcttttctaatgtgacctggttccacgcaatccacgtgagcggcagcaacggcacgaaatatttcgataatcctgtgctgcccttcaacgacggcgtgtacttcgcctccacagagaagtcaaacattattcgggggtggatcttcggcaccacactggacagtaaaacccagtccctgctgatcgtgaacaatgctaccaacgtggtgattaaggtgtgctactttcagttctgcaatgatccattcctggatgtgtactaccacaagaacaataagtcctggatggaaagcgaattcagagtgtattcctctgcaaataactgtacattcgagtacgtgtccaagccattcctcatggaccttgaaggcaagcaggggaattttaaaaacctgcgggagtttgtgtttaagaatatcgatggctacttcaagatttattccaaacacactcccatcaacctgaccagggacctgccccagggcttttccgccctggagcccctggtggacctgcccattggcattaacattacaagattccagaccctgctggctctgcacaggtcctatctgacacctggcgatagcagcaatggctggaccgtcggggcagccgcctattacgtgggatacctgcagccccggacctttctgctgaagtacaatgagaacggcacaatcacagacgctgtggactgcgccctggacccactgagcgagactaagtgcactctgaaatcattcactgtggagaaggggatttatcagacatctaacttcagagtccagcctaccgagagtatcgtgaggttccccaacatcaccaacctgtgcccttttcacgaggtgtttaacgccaccaccttcgcctcagtgtatgcctggaacaggaagagaatcagcaattgcgtggcagactactccgtgctgtacaacttcgcacccttcttcgcctttaagtgttacggcgtgagccccaccaaactgaacgatctgtgcttcacaaatgtgtacgccgatagttttgtgatccgggggaatgaggtgtctcaaatcgcccccggccagaccggaaacatcgctgattataactacaagctgcccgatgattttaccgggtgcgtgatcgcctggaatagtaataagctggatagcaaagtgtctggcaactataactatctgtaccggctgttcaggaaatccaagctgaagcccttcgagcgcgacatctctaccgaaatttaccaggccggtaacaagccatgtaacggcgtggctggctttaattgctacttcccactgcagagctacgggttccggcccacctatggggtggggcatcagccctaccgcgtggtggtgctgtccttcgagctgctgcacgccccagctaccgtgtgcgggcccaagaagtcaactaatctggtcaagaacaagtgtgtcaacttcaacttcaatggcctgactgggacaggcgtgctcaccgagagcaacaagaaattcctgccttttcagcagttcggcagggacatcgccgacaccacagatgccgttcgggacccacagaccctggaaatcctggatattactccatgcagcttcggcggcgtgtccgtgattacacctggaaccaacaccagcaaccaggtggccgtgctgtaccagggcgtgaactgcaccgaggtgcctgtggcaatacacgccgaccagctgactcccacatggcgagtgtattcaaccggctccaatgtgtttcagactagagccggctgcctgatcggagctgagcacgtgaataactcttatgagtgcgatatccccatcggagccggcatctgcgcatcctatcagacacagacaaacagccacgccagcgtggcctctcaaagcatcattgcctacacaatgagcctgggcgccgagaactccgttgcttatagtaataatagcattgcaatccctaccaacttcaccatcagcgtgaccacagagattctgcctgtgagcatgaccaagacctccgtggactgtaccatgtacatctgtggcgactcgactgaatgcagcaacctgctgctccaatacggcagcttctgcacacagctgaaccgggctctgaccggcatcgccgtggaacaggacaagaacacccaagaggtgtttgcccaggtgaagcagatttacaagacccctcccatcaaggacttcggcggtttcaactttagccagatcctgcctgatccttctaaacccagcaagcggagccccatcgaggacctgctgttcaacaaggtgaccctggctgacgccggattcatcaagcagtatggcgactgtctgggcgatatcgctgccagagacctcatctgtgcccagaagttcaacggactgactgtgctaccacccttactgaccgatgagatgatcgctcagtacacatctgccctgctggctgggaccatcaccagcggctggacattcggcgccggccccgccctgcaaatcccgtttcctatgcagatggcctatagattcaacggaatcggcgtgacccagaacgtcctgtatgagaaccagaagctgatcgctaatcagttcaacagcgcaatcggcaaaatccaagactctctgagcagcacaccctccgctctgggcaagctgcaggatgtggtgaaccaaaacgcccaagcccttaacaccctggtgaaacagctgagcagcaactttggtgccatcagcagcgtgcttaacgacattctgtctaggctggacccccctgaggccgaagtgcagatcgacagactgatcaccggaagactgcagagcctgcaaacctacgtgacccaacagttgatcagagccgctgagatccgggcgagcgccaacctggccgccacaaagatgagcgaatgtgtgctggggcagagcaaaagagtggacttctgcggcaaaggctatcacctgatgagctttcctcagagcgccccccacggcgtggtgttcctgcacgtgacatacgtgcctgctcaggagaagaacttcaccaccgcccctgccatctgccacgacggcaaggctcacttcccaagagaaggcgtgttcgtcagcaatggcacccactggttcgtgacccaaagaaacttctacgagcctcagatcatcacaaccgacaacacattcgtgagcgggaactgcgacgtggtaatcggcattgtgaacaataccgtgtacgaccctctgcagccagaactggattccttcaaggaagagctggacaagtactttaagaaccacacaagccctgacgtggacctgggcgacataagcggtatcaacgcctcagtggtcaacatccagaaagagatcgacaggttaaacgaagtggccaagaacctgaatgagtccctgatcgatctgcaggagctgggcaagtacgaacagtacatcaagtggccttggtacatctggctgggattcattgccggattgatcgccatcgtcatggtgaccatcatgctgtgctgcatgacgagctgctgctcttgcctgaagggctgctgtagctgtggctcctgttgtaaattcgacgaagatgacagcgagcccgtgctgaagggagtgaagctgcattacacc

SEQ ID NO: 14 - DNA, 3810 bp, SARS-CoV-2 Omicron JN.1
  source 1..3810: mol_type = genomic DNA
atgtttgtttttcttgttttattgccactagtctctagtcagtgtgtcatgccgctgtttaatcttataactacaactcaatcatacactaattctttcacacgtggtgtttattaccctgacaaagttttcagatcctcagttttacatttaactcaggacttgttcttacctttcttttccaatgttacttggttccatgctatctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgtttttattaaagtctgtgaatttcaattttgtaatgatccatttttgggtgtttaccacaaaaacaacaaaagttggatggaaagtgagtcaggagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattatagggcgtgatttccctcagggtttttcggctttagaaccattggtagatttgccaataggtattaacatcactaggtttcaaactttacttgctttaaatagaagttatttgactcctggtgattcttcttcaggttggacagctggtgctgcagattattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatgttacaaacttgtgcccttttcatgaagtttttaacgccaccagatttgcatctgtttatgcttggaacaggacgagaatcagcaactgtgttgctgattattctgtcctatataatttcgcaccatttttcgcttttaagtgttatggagtgtctcctactaaattaaatgatctctgctttactaatgtctatgcagattcatttgtaattaaaggtaatgaagtcagccaaatcgctccagggcaaactggaaatattgctgattataattataaattaccagatgattttacaggctgcgttatagcttggaattctaacaagcttgattctaagcatagtggtaattatgattactggtatagatcgtttaggaagtctaaactcaaaccttttgagagagatatttcaactgaaatctatcaggccggtaacaaaccttgtaaaggtaaaggtcctaattgttactttcctttacaatcatatggtttccgacccacttatggtgttggtcaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactaagtctaacaaaaagtttctgcctttccaacaatttggcagagacattgttgacactactgatgctgtccgtgatccacagacacttgagattcttgacattacaccatgttcttttggtggtgtcagtgttataacaccaggaacaaatacttctaaccaggttgctgttctttatcagggtgttaactgcacagaagtctctgttgctattcatgcagatcaacttactcctacttggcgtgtttattctacaggttctaatgtttttcaaacacgtgcaggctgtttaataggggctgaatatgtcaacaactcatatgagtgtgacatacccattggtgcaggtatatgcgctagttatcagactcagactaagtctcgtcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaacgtgctttaactggaatagctgttgaacaagacaaaaacacccaagaagtttttgcacaagtcaaacaaatttacaaaacaccaccaattaaatattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcacttttttccacagcaagtgcacttggaaaacttcaagatgtggtcaaccataatgcacaagctttaaacacgcttgttaaacaacttagctccaaatttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacttgaattagattcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtatatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 15 - DNA, 3807 bp, SARS-CoV-2 Omicron JN.1
  source 1..3807: mol_type = genomic DNA
atgtttgtgttcctcgtattacttccgctggtgtcgagccagtgcgtgatgccgttgttcaacctgatcacgacaacacagagctacacgaacagctttactcggggtgtgtattaccccgataaagtgttccgtagctctgtgttgcatctgactcaggacctcttcctcccgttcttctccaatgtcacgtggttccacgcgatatcggggacgaacgggacgaagaggttcgacaaccctgttctgccgttcaacgacggggtgtacttcgcttcgacagagaagtccaacatcatccggggctggatcttcggtaccaccctagatagtaagacgcagtccctgctgatcgtgaacaacgctactaacgtgttcatcaaggtgtgtgagttccagttctgtaacgacccctttctcggagtctatcacaagaacaacaagtcttggatggagtccgagagcggggtctacagttctgcgaacaactgcacctttgaatacgttagtagcccgttcttgatggatctcgaggggaagcagggcaactttaaaaacctgcgggaattcgtgttcaagaatatcgacgggtacttcaagatctactcgaagcataccccgataattggacgcgatttcccgcaggggtttagtgccctggagcccctcgtagatcttccaatcggaattaatatcacccggtttcagacgctcctggcgctgaacaggagctatctgacgccgggtgatagtagttccggttggacagcaggggctgcggattactatgtagggtacctccagccccggacgttcttgttgaagtacaacgagaacggcacaatcaccgacgcggttgattgtgcgttggaccctctgtcggagacgaagtgcaccctgaagtcgtttacggtagaaaaggggatctatcagacctccaacttccgcgtccagccgactgagagtatcgttcggttcccgaacgttactaatctgtgtccgttccacgaggtattcaacgctacgcggttcgcgagcgtgtacgcgtggaaccggacacggattagtaactgtgtagcagactacagtgtgctatacaacttcgccccgttcttcgcgttcaagtgttacggggtgtcgcccacgaagttgaacgacctctgcttcaccaacgtgtacgccgacagctttgtgattaagggcaacgaggtctcgcagatcgccccagggcagacggggaacattgctgactacaactacaagctgcctgacgattttaccggttgcgttattgcctggaatagcaataagcttgacagcaagcattccggaaactacgattactggtatcgtagtttccggaagagcaagctgaagcccttcgagagggatatctctacggagatataccaggccggaaacaagccctgcaaggggaaggggccgaactgctacttccccttgcagagctacggtttccggcctacctacggggtagggcaccagccctaccgcgtagtggtgctctccttcgagctgctccacgcaccggcgacggtgtgtgggccgaagaagagcaccaatctcgtgaagaacaaatgcgtcaacttcaatttcaatggactgactggaacgggagtgctcacgaagagcaacaagaagttcctcccgttccagcagttcgggcgtgatatcgttgacaccaccgatgctgtgcgtgatccccagactctggagattctggatatcacgccgtgcagcttcggtggtgtcagcgttatcacgcccggaacgaatacatccaaccaggtggcggtgctgtatcagggcgtgaattgcaccgaggtatcggttgcaattcacgccgatcagctgacccccacctggcgggtgtattccacgggcagcaatgtcttccagacacgcgccggctgcttgataggggcagagtatgtgaataatagctacgagtgcgacattccgatcggcgccgggatttgtgcgtcgtaccagacgcagaccaagtcccggcgccgagctcggagtgtcgcctcgcagtctattattgcatatactatgtccctgggagccgagaactcagtagcatattcgaacaactccattgctatccccacaaattttacaattagtgtaaccaccgagatcttgccggtctcgatgaccaagacctcggtggattgcactatgtatatttgtggggatagcacggagtgttcgaatttgctgctgcagtacggctccttctgcactcagctgaagcgagcactgactgggattgctgtcgagcaggacaagaatacccaggaggtgttcgctcaggtgaagcagatctacaagacgcccccgatcaagtacttcgggggcttcaacttcagccagattctgccagacccatctaagccgagcaagaggtcctttattgaggacctcttgttcaacaaggtgactctggcagatgctggcttcatcaagcagtacggcgactgtctgggagacattgctgcccgtgacctcatctgcgcgcagaagttcaacggtctgacagtgctgcccccgctcctcaccgacgagatgatcgcccagtacaccagcgcgctgctggctgggacgatcacctcggggtggaccttcggagcgggggcagcactgcagatcccttttgcgatgcagatggcgtatcggttcaatggaattggagtgacgcagaatgttctttacgagaaccagaagcttattgctaaccagttcaatagcgcaatcggtaagatccaggacagcttgttcagcaccgcgtctgccctggggaagctgcaagacgtcgttaatcacaatgctcaggcgttgaacacgttggtgaagcagttgtcgtccaagttcggggcgatcagttcggtgctgaacgatattctcagtcggctggacaaggtggaagcggaggtccagatagatcggctcatcactggtcgcctccagagtttgcagacgtacgtaactcagcagctcatccgagctgctgagatacgtgcgtctgcaaacctggcggcgaccaagatgagcgagtgcgtgcttggacagtccaagcgcgtagacttctgcgggaagggctatcacctgatgtccttcccgcagagcgccccccacggggtggtcttcctgcacgtgacatatgtgccggcgcaggagaagaacttcacgactgcgccggccatatgtcacgacgggaaggcccacttcccccgtgagggggtgttcgtgagcaacggcacgcactggttcgtcacccagcggaacttttacgagccacagataattaccactgacaatacctttgtcagtggtaactgcgacgtggtcataggcattgtgaacaacactgtctatgacccgttgcagttggagcttgactcctttaaggaggagctcgacaagtacttcaagaatcatacctcgccggacgttgatctcggagacatctccgggatcaacgcttcggtggtcaatatccagaaggagattgaccgcctcaacgaggtggccaagaaccttaatgaatcgctcatagatctccaggagctggggaagtatgagcagtacattaagtggccttggtacatctggttggggtttatagcagggctgatcgcgatcgtgatggtcacgatcatgctctgctgtatgacgagctgctgcagctgcctcaagggctgctgctcttgtggcagctgctgcaagttcgacgaggatgattccgagcccgtccttaaaggagtcaagctccactacacg

SEQ ID NO: 16 - DNA, 3807 bp, SARS-CoV-2 Omicron JN.1
  source 1..3807: mol_type = genomic DNA
atgtttgtgttcctcgtattacttccgctggtgtcgagccagtgcgtgatgccgttgttcaacctgatcacgacaacacagagctacacgaacagctttactcggggtgtgtattaccccgataaagtgttccgtagctctgtgttgcatctgactcaggacctcttcctcccgttcttctccaatgtcacgtggttccacgcgatatcggggacgaacgggacgaagaggttcgacaaccctgttctgccgttcaacgacggggtgtacttcgcttcgacagagaagtccaacatcatccggggctggatcttcggtaccaccctagatagtaagacgcagtccctgctgatcgtcaataacgcgactaacgtgttcatcaaggtgtgtgagttccagttctgtaacgacccctttctcggagtctatcacaagaacaacaagtcttggatggagtccgagagcggggtctacagttctgcgaacaactgcacctttgaatacgttagtcagccgtttttgatggacctcgaggggaagcagggcaactttaaaaacctgcgggaattcgtgttcaagaatatcgacgggtacttcaagatctactcgaagcataccccgataattggacgcgatttcccgcaggggtttagtgccctggagcccctcgtggatctacccattgggatcaatatcacccggtttcagacgctcctggcgctgaacaggagctatctgacgccgggtgatagttcccaagggtggacagcaggggctgcggattactatgtagggtacctccagccccggacgttcttgttgaagtacaacgagaacggcacaatcaccgacgcggttgattgtgcgttggaccctctgtcggagacgaagtgcaccctgaagtcgtttacggtagaaaaggggatctatcagacctccaacttccgcgtccagccgactgagagtatcgttcggttcccgaacgttactaatctgtgtccgttccacgaggtattcaacgctacgcggttcgcgagcgtgtacgcgtggaaccggacacggattagtaactgtgtagcagactacagtgtgctatacaacttcgccccgttcttcgcgttcaagtgttacggggtgtcgcccacgaagttgaacgacctctgcttcaccaacgtgtacgccgacagctttgtgattaagggcaacgaggtctcgcagatcgccccagggcagacggggaacattgctgactacaactacaagctgcctgacgattttaccggttgcgttattgcctggaatagcaataagcttgacagcaagcattccggaaactacgattactggtatcgtagtttccggaagagcaagctgaagcccttcgagagggatatctctacggagatataccaggccggaaacaagccctgcaaggggaaggggccgaactgctacttccccttgcagagctacggtttccggcctacctacggggtagggcaccagccctaccgcgtagtggtgctctccttcgagctgctccacgcaccggcgacggtgtgtgggccgaagaagagcaccaatctcgtgaagaacaaatgcgtcaacttcaatttcaatggactgactggaacgggagtgctcacgaagagcaacaagaagttcctcccgttccagcagttcgggcgtgatatcgttgacaccaccgatgctgtgcgtgatccccagactctggagattctggatatcacgccgtgcagcttcggtggtgtcagcgttatcacgcccggaacgaatacatccaaccaggtggcggtgctgtatcagggcgtgaattgcaccgaggtatcggttgcaattcacgccgatcagctgacccccacctggcgggtgtattccacgggcagcaatgtcttccagacacgcgccggctgcttgataggggcagagtatgtgaataatagctacgagtgcgacattccgatcggcgccgggatttgtgcgtcgtaccagacgcagaccaagtcccggcgccgagctcggagtgtcgcctcgcagtctattattgcatatactatgtccctgggagccgagaactcagtagcatattcgaacaactccattgctatccccacaaattttacaattagtgtaaccaccgagatcttgccggtctcgatgaccaagacctcggtggattgcactatgtatatttgtggggatagcacggagtgttcgaatttgctgctgcagtacggctccttctgcactcagctgaagcgagcactgactgggattgctgtcgagcaggacaagaatacccaggaggtgttcgctcaggtgaagcagatctacaagacgcccccgatcaagtacttcgggggcttcaacttcagccagattctgccagacccatctaagccgagcaagaggtcctttattgaggacctcttgttcaacaaggtgactctggcagatgctggcttcatcaagcagtacggcgactgtctgggagacattgctgcccgtgacctcatctgcgcgcagaagttcaacggtctgacagtgctgcccccgctcctcaccgacgagatgatcgcccagtacaccagcgcgctgctggctgggacgatcacctcggggtggaccttcggagcgggggcagcactgcagatcccttttgcgatgcagatggcgtatcggttcaatggaattggagtgacgcagaatgttctttacgagaaccagaagcttattgctaaccagttcaatagcgcaatcggtaagatccaggacagcttgttcagcaccgcgtctgccctggggaagctgcaagacgtcgttaatcacaatgctcaggcgttgaacacgttggtgaagcagttgtcgtccaagttcggggcgatcagttcggtgctgaacgatattctcagtcggctggacaaggtggaagcggaggtccagatagatcggctcatcactggtcgcctccagagtttgcagacgtacgtaactcagcagctcatccgagctgctgagatacgtgcgtctgcaaacctggcggcgaccaagatgagcgagtgcgtgcttggacagtccaagcgcgtagacttctgcgggaagggctatcacctgatgtccttcccgcagagcgccccccacggggtggtcttcctgcacgtgacatatgtgccggcgcaggagaagaacttcacgactgcgccggccatatgtcacgacgggaaggcccacttcccccgtgagggggtgttcgtgagcaacggcacgcactggttcgtcacccagcggaacttttacgagccacagataattaccactgacaatacctttgtcagtggtaactgcgacgtggtcataggcattgtgaacaacactgtctatgacccgttgcagttggagcttgactcctttaaggaggagctcgacaagtacttcaagaatcatacctcgccggacgttgatctcggagacatctccgggatcaacgcttcggtggtcaatatccagaaggagattgaccgcctcaacgaggtggccaagaaccttaatgaatcgctcatagatctccaggagctggggaagtatgagcagtacattaagtggccttggtacatctggttggggtttatagcagggctgatcgcgatcgtgatggtcacgatcatgctctgctgtatgacgagctgctgcagctgcctcaagggctgctgctcttgtggcagctgctgcaagttcgacgaggatgattccgagcccgtccttaaaggagtcaagctccactacacg

SEQ ID NO: 17 - DNA, 3807 bp, SARS-CoV-2 Omicron JN.1
  source 1..3807: mol_type = genomic DNA
atgtttgtgttcctcgtattacttccgctggtgtcgagccagtgcgtgatgccgttgttcaacctgatcacgacaacacagagctacacgaacagctttactcggggtgtgtattaccccgataaagtgttccgtagctctgtgttgcatctgactcaggacctcttcctcccgttcttctccaatgtcacgtggttccacgcgatatcggggacgaacgggacgaagaggttcgacaaccctgttctgccgttcaacgacggggtgtacttcgcttcgacagagaagtccaacatcatccggggctggatcttcggtaccaccctagatagtaagacgcagtccctgctgatcgtgaacaacgctactaacgtgttcatcaaggtgtgtgagttccagttctgtaacgacccctttctcggagtctatcacaagaacaacaagtcttggatggagtccgagagcggggtctacagttctgcgaacaactgcacctttgaatacgttagtagcccgttcttgatggacctggagggcaagcaggggaacttcaagaacctgcgggaattcgtgttcaagaatatcgacgggtacttcaagatctactcgaagcataccccgataattggacgcgatttcccgcaggggttctccgcgctggagccgctggtggacttgccgataggcatcaacatcacgcggttccagacgctgcttgccctcaacaggtcctatctcacccctggggactcttcccaggggtggacagcaggggctgcggattactatgtagggtacctccagccccggacgttcttgttgaagtacaacgagaacggcacaatcaccgacgcggttgattgtgcgttggaccctctgtcggagacgaagtgcaccctgaagtcgtttacggtagaaaaggggatctatcagacctccaacttccgcgtccagccgactgagagtatcgttcggttcccgaacgttactaatctgtgtccgttccacgaggtattcaacgctacgcggttcgcgagcgtgtacgcgtggaaccggacacggattagtaactgtgtagcagactacagtgtgctatacaacttcgccccgttcttcgcgttcaagtgttacggggtgtcgcccacgaagttgaacgacctctgcttcaccaacgtgtacgccgacagctttgtgattaagggcaacgaggtctcgcagatcgccccagggcagacggggaacattgctgactacaactacaagctgcctgacgattttaccggttgcgttattgcctggaatagcaataagcttgacagcaagcattccggaaactacgattactggtatcgtagtttccggaagagcaagctgaagcccttcgagagggatatctctacggagatataccaggccggaaacaagccctgcaaggggaaggggccgaactgctacttccccttgcagagctacggtttccggcctacctacggggtagggcaccagccctaccgcgtagtggtgctctccttcgagctgctccacgcaccggcgacggtgtgtgggccgaagaagagcaccaatctcgtgaagaacaaatgcgtcaacttcaatttcaatggactgactggaacgggagtgctcacgaagagcaacaagaagttcctcccgttccagcagttcgggcgtgatatcgttgacaccaccgatgctgtgcgtgatccccagactctggagattctggatatcacgccgtgcagcttcggtggtgtcagcgttatcacgcccggaacgaatacatccaaccaggtggcggtgctgtatcagggcgtgaattgcaccgaggtatcggttgcaattcacgccgatcagctgacccccacctggcgggtgtattccacgggcagcaatgtcttccagacacgcgccggctgcttgataggggcagagtatgtgaataatagctacgagtgcgacattccgatcggcgccgggatttgtgcgtcgtaccagacgcagaccaagtcccggcgccgagctcggagtgtcgcctcgcagtctattattgcatatactatgtccctgggagccgagaactcagtagcatattcgaacaactccattgctatccccacaaattttacaattagtgtaaccaccgagatcttgccggtctcgatgaccaagacctcggtggattgcactatgtatatttgtggggatagcacggagtgttcgaatttgctgctgcagtacggctccttctgcactcagctgaagcgagcactgactgggattgctgtcgagcaggacaagaatacccaggaggtgttcgctcaggtgaagcagatctacaagacgcccccgatcaagtacttcgggggcttcaacttcagccagattctgccagacccatctaagccgagcaagaggtcctttattgaggacctcttgttcaacaaggtgactctggcagatgctggcttcatcaagcagtacggcgactgtctgggagacattgctgcccgtgacctcatctgcgcgcagaagttcaacggtctgacagtgctgcccccgctcctcaccgacgagatgatcgcccagtacaccagcgcgctgctggctgggacgatcacctcggggtggaccttcggagcgggggcagcactgcagatcccttttgcgatgcagatggcgtatcggttcaatggaattggagtgacgcagaatgttctttacgagaaccagaagcttattgctaaccagttcaatagcgcaatcggtaagatccaggacagcttgttcagcaccgcgtctgccctggggaagctgcaagacgtcgttaatcacaatgctcaggcgttgaacacgttggtgaagcagttgtcgtccaagttcggggcgatcagttcggtgctgaacgatattctcagtcggctggacaaggtggaagcggaggtccagatagatcggctcatcactggtcgcctccagagtttgcagacgtacgtaactcagcagctcatccgagctgctgagatacgtgcgtctgcaaacctggcggcgaccaagatgagcgagtgcgtgcttggacagtccaagcgcgtagacttctgcgggaagggctatcacctgatgtccttcccgcagagcgccccccacggggtggtcttcctgcacgtgacatatgtgccggcgcaggagaagaacttcacgactgcgccggccatatgtcacgacgggaaggcccacttcccccgtgagggggtgttcgtgagcaacggcacgcactggttcgtcacccagcggaacttttacgagccacagataattaccactgacaatacctttgtcagtggtaactgcgacgtggtcataggcattgtgaacaacactgtctatgacccgttgcagttggagcttgactcctttaaggaggagctcgacaagtacttcaagaatcatacctcgccggacgttgatctcggagacatctccgggatcaacgcttcggtggtcaatatccagaaggagattgaccgcctcaacgaggtggccaagaaccttaatgaatcgctcatagatctccaggagctggggaagtatgagcagtacattaagtggccttggtacatctggttggggtttatagcagggctgatcgcgatcgtgatggtcacgatcatgctctgctgtatgacgagctgctgcagctgcctcaagggctgctgctcttgtggcagctgctgcaagttcgacgaggatgattccgagcccgtccttaaaggagtcaagctccactacacg

SEQ ID NO: 18 - DNA, 3807 bp, SARS-CoV-2 Omicron JN.1
  source 1..3807: mol_type = genomic DNA
atgtttgtgttcctcgtattacttccgctggtgtcgagccagtgcgtgatgccgttgttcaacttgatcacgacgactcaggagtacactaatagtttcactcggggggtctactacccggataaagtatttcggtcgtcagtgctgcacatgacgaccgatttatttcttccgttcttctccaacgtgacgtggtttcacgccatcagcgggagcaacgggacgaagtacttcgacaatcccgtgctcccgttcaatgatggcgtgtacttcgcgtcaacggagaagagcaacatcatccggggctggatcttcggtaccaccctagatagtaagacgcagtccctgctgatcgtgaacaacgctactaacgtgttcatcaaggtgtgttatttccagttttgtaacgacccctttctcggagtctatcacaagaacaacaagtcttggatggagtccgagagcggggtctacgattctgcgaataactgcacctttgaatacgttagtagcccgttcttgatggacctggagggcaagcaggggaacttcaagaacctgcgtgagtttgtgttcaagaacatcgatgggtacttcaagatctactcgaagcacacgccgataatcacgcgggacttcccgcagggattttcggcgctggagcctctggtagatctgcccatcggcataaacatcacgcggttccagaccctgcttgccctcaacaggtcctatctcacccctggggactcttcccaggggtggacagtaggggctgcggattactatgtagggtacctccagccccggacgttcttgttgaagtacaacgagaacggcacaatcaccgacgcggttgattgtgccctagaccccctgagtgagactaagtgtaccctgaagtcgttcacggtcgagaaggggatctatcagacctccaacttccgcgtccagccgactgacagtatcgttcggttcccgaacgttactaatctgtgtccgttccacgaggtattcaacgctacgcggttcgcgagcgtgtacgcgtggaaccggacacggattagtaactgtgtagcagactacagtgtgctatacaacttcgccccgttcttcgcgttcaagtgttacggggtgtcgcccacgaagttgaacgacctctgcttcaccaacgtgtacgccgacagctttgtgattaagggcaacgaggtctcgcagatcgccccagggcagacggggaacattgctgactacaactacaagctgcctgacgattttaccggttgcgttattgcctggaatagcaataagcttgacagcaagcattccggaaactacgattactggtatcgtagtttccggaagagcaagctgaagcccttcgagagggatatctctacggagatataccaggccggaaacaagccctgcaaggggaaggggccgaactgctacttccccttgcagagctacggtttccggcctacctacggggtagggcaccagccctaccgcgtagtggtgctctccttcgagctgctccacgcaccggcgacggtgtgtgggccgaagaagagcaccaatctcgtgaagaacaaatgcgtcaacttcaatttcaatggactgactggaacgggagtgctcacgaagagcaacaagaagttcctcccgttccagcagttcgggcgtgatatcgttgacaccaccgatgctgtgcgtgatccccagactctggagattctggatatcacgccgtgcagcttcggtggtgtcagcgttatcacgcccggaacgaatacatccaaccaggtggcggtgctgtatcagggcgtgaattgcaccgaggtatcggttgcaattcacgccgatcagctgacccccacctggcgggtgtattccacgggcagcaatgtcttccagacacgcgccggctgcttgataggggcagagtatgtgaataatagctacgagtgcgacattccgatcggcgccgggatttgtgcgtcgtaccagacgcagaccaagtcccggcgccgagctcggagtgtcgcctcgcagtctattattgcatatactatgtccctgggagccgagaactcagtagcatattcgaacaactccattgctatccccacaaattttacaattagtgtaaccaccgagatcttgccggtctcgatgaccaagacctcggtggattgcactatgtatatttgtggggatagcacggagtgttcgaatttgctgctgcagtacggctccttctgcactcagctgaagcgagcactgactgggattgctgtcgagcaggacaagaatacccaggaggtgttcgctcaggtgaagcagatctacaagacgcccccgatcaagtacttcgggggcttcaacttcagccagattctgccagacccatctaagccgagcaagaggtcctttattgaggacctcttgttcaacaaggtgactctggcagatgctggcttcatcaagcagtacggcgactgtctgggagacattgctgcccgtgacctcatctgcgcgcagaagttcaacggtctgacagtgctgcccccgctcctcaccgacgagatgatcgcccagtacaccagcgcgctgctggctgggacgatcacctcggggtggaccttcggagcgggggcagcactgcagatcccttttgcgatgcagatggcgtatcggttcaatggaattggagtgacgcagaatgttctttacgagaaccagaagcttattgctaaccagttcaatagcgcaatcggtaagatccaggacagcttgttcagcaccgcgtctgccctggggaagctgcaagacgtcgttaatcacaatgctcaggcgttgaacacgttggtgaagcagttgtcgtccaagttcggggcgatcagttcggtgctgaacgatattctcagtcggctggacaaggtggaagcggaggtccagatagatcgccttatcactggtcgcctccagagtttgcagacgtacgtaactcagcagctcatccgagctgctgagatacgtgcgtctgcaaacctggcggcgaccaagatgtccgagtgcgtgctcgggcagagcaagcgcgtggacttctgcgggaagggctatcacctgatgtccttcccgcagagcgccccccacggggtggtcttcctgcacgtgacatatgtgccggcgcaggagaagaacttcacgactgcgccggccatatgtcacgacgggaaggcccacttcccccgtgagggggtgttcgtgagcaacggcacgcactggttcgtcacccagcggaacttttacgagccacagataattaccactgacaatacctttgtcagtggtaactgcgacgtggtcataggcattgtgaacaacactgtctatgacccgttgcagttggagcttgactcctttaaggaggagctcgacaagtacttcaagaatcatacctcgccggacgttgatctcggagacatctccgggatcaacgcttcggtggtcaatatccagaaggagattgaccgcctcaacgaggtggccaagaaccttaatgaatcgctcatagatctccaggagctggggaagtatgagcagtacattaagtggccttggtacatctggttggggtttatagcagggctgatcgcgatcgtgatggtcacgatcatgctctgctgtatgacgagctgctgcagctgcctcaagggctgctgctcttgtggcagctgctgcaagttcgacgaggatgattccgagcccgtccttaaaggagtcaagctccactacacg

SEQ ID NO: 19 - DNA, 3807 bp, SARS-CoV-2 Omicron JN.1
  source 1..3807: mol_type = genomic DNA
atgtttgtgttcctcgtattacttccgctggtgtcgagccagtgcgtgatgccgttgttcaatctcattactactactcaggagtatacgaactcgttcacccggggggtgtattacccggataaggtattcaggagcagcgtccttcacatgacgacggacctgttcctgccgttcttctccaacgtgacgtggtttcacgccatcagcgggagcaacgggacgaagtacttcgacaatcccgtgctcccgttcaatgatggcgtgtacttcgcgtcaacggagaagagcaatatcatcaggggctggatcttcgggaccacgctggactcgaagacccagtccctgttgatagtcaataacgcgactaacgtgttcatcaaggtgtgttatttccagttttgtaacgacccctttctcggagtctatcacaagaacaacaagtcttggatggagtccgagagcggggtctacgattctgcgaataactgcacctttgaatacgttagtcagccgtttttgatggacctggagggcaagcaggggaacttcaagaacctgcgtgagtttgtgttcaagaacatcgatgggtacttcaagatctactcgaagcacacgccgataatcacgcgggacttcccgcagggattttcggcgctggagcctctggtagatctgcccatcggcataaacatcacgcggttccagaccctgcttgccctcaacaggtcctaccttactccgggtgattcatcctccgggtggacggtcggggctgcagattactacgttgggtatctgcagccccgaacgtttctcctgaagtataatgagaacggcacaatcaccgacgcggttgattgtgccctcgacccgcttagtgagaccaagtgcacactcaagtcatttacggtggagaaggggatctatcagacctccaacttccgcgtccagccgactgacagtatcgttcggttcccgaacgttactaatctgtgtccgttccacgaggtattcaacgctacgcggttcgcgagcgtgtacgcgtggaaccggacacggattagtaactgtgtagcagactacagtgtgctatacaacttcgccccgttcttcgcgttcaagtgttacggggtgtcgcccacgaagttgaacgacctctgcttcaccaacgtgtacgccgacagctttgtgattaagggcaacgaggtctcgcagatcgccccagggcagacggggaacattgctgactacaactacaagctgcctgacgattttaccggttgcgttattgcctggaatagcaataagcttgacagcaagcattccggaaactacgattactggtatcgtagtttccggaagagcaagctgaagcccttcgagagggatatctctacggagatataccaggccggaaacaagccctgcaaggggaaggggccgaactgctacttccccttgcagagctacggtttccggcctacctacggggtagggcaccagccctaccgcgtagtggtgctctccttcgagctgctccacgcaccggcgacggtgtgtgggccgaagaagagcaccaatctcgtgaagaacaaatgcgtcaacttcaatttcaatggactgactggaacgggagtgctcacgaagagcaacaagaagttcctcccgttccagcagttcgggcgtgatatcgttgacaccaccgatgctgtgcgtgatccccagactctggagattctggatatcacgccgtgcagcttcggtggtgtcagcgttatcacgcccggaacgaatacatccaaccaggtggcggtgctgtatcagggcgtgaattgcaccgaggtatcggttgcaattcacgccgatcagctgacccccacctggcgggtgtattccacgggcagcaatgtcttccagacacgcgccggctgcttgataggggcagagtatgtgaataatagctacgagtgcgacattccgatcggcgccgggatttgtgcgtcgtaccagacgcagaccaagtcccggcgccgagctcggagtgtcgcctcgcagtctattattgcatatactatgtccctgggagccgagaactcagtagcatattcgaacaactccattgctatccccacaaattttacaattagtgtaaccaccgagatcttgccggtctcgatgaccaagacctcggtggattgcactatgtatatttgtggggatagcacggagtgttcgaatttgctgctgcagtacggctccttctgcactcagctgaagcgagcactgactgggattgctgtcgagcaggacaagaatacccaggaggtgttcgctcaggtgaagcagatctacaagacgcccccgatcaagtacttcgggggcttcaacttcagccagattctgccagacccatctaagccgagcaagaggtcctttattgaggacctcttgttcaacaaggtgactctggcagatgctggcttcatcaagcagtacggcgactgtctgggagacattgctgcccgtgacctcatctgcgcgcagaagttcaacggtctgacagtgctgcccccgctcctcaccgacgagatgatcgcccagtacaccagcgcgctgctggctgggacgatcacctcggggtggaccttcggagcgggggcagcactgcagatcccttttgcgatgcagatggcgtatcggttcaatggaattggagtgacgcagaatgttctttacgagaaccagaagcttattgctaaccagttcaatagcgcaatcggtaagatccaggacagcttgttcagcaccgcgtctgccctggggaagctgcaagacgtcgttaatcacaatgctcaggcgttgaacacgttggtgaagcagttgtcgtccaagttcggggcgatcagttcggtgctgaacgatattctcagtcggctggacaaggtggaagcggaggtccagatagatcgccttatcaccggtcgcctccagagtttgcagacgtacgtaactcagcagctcatccgagctgctgagatacgtgcgtctgcaaacctggcggcgaccaagatgagtgagtgtgtacttggtcagtctaagcgggtcgacttctgcgggaagggctatcacctgatgtccttcccgcagagcgccccccacggggtggtcttcctgcacgtgacatatgtgccggcgcaggagaagaacttcacgactgcgccggccatatgtcacgacgggaaggcccacttcccccgtgagggggtgttcgtgagcaacggcacgcactggttcgtcacccagcggaacttttacgagccacagataattaccactgacaatacctttgtcagtggtaactgcgacgtggtcataggcattgtgaacaacactgtctatgacccgttgcagttggagcttgactcctttaaggaggagctcgacaagtacttcaagaatcatacctcgccggacgttgatctcggagacatctccgggatcaacgcttcggtggtcaatatccagaaggagattgaccgcctcaacgaggtggccaagaaccttaatgaatcgctcatagatctccaggagctggggaagtatgagcagtacattaagtggccttggtacatctggttggggtttatagcagggctgatcgcgatcgtgatggtcacgatcatgctctgctgtatgacgagctgctgcagctgcctcaagggctgctgctcttgtggcagctgctgcaagttcgacgaggatgattccgagcccgtccttaaaggagtcaagctccactacacg

SEQ ID NO: 20 - protein, 1269 aa, SARS-CoV-2 Omicron JN.1
MFVFLVLLPLVSSQCVMPLFNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLGVYHKNNKSWMESESGVYSSANNCTFEYVSQPFLMDLEGKQGNFKNLREFVFKNIDGYFKIYSKHTPIIGRDFPQGFSALEPLVDLPIGINITRFQTLLALNRSYLTPGDSSSGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGICASYQTQTKSRRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 21 - protein, 1269 aa, SARS-CoV-2 Omicron JN.1
MFVFLVLLPLVSSQCVMPLFNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLGVYHKNNKSWMESESGVYSSANNCTFEYVSSPFLMDLEGKQGNFKNLREFVFKNIDGYFKIYSKHTPIIGRDFPQGFSALEPLVDLPIGINITRFQTLLALNRSYLTPGDSSSGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGICASYQTQTKSRRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - protein, 1269 aa, SARS-CoV-2 Omicron JN.1
MFVFLVLLPLVSSQCVMPLFNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLGVYHKNNKSWMESESGVYSSANNCTFEYVSQPFLMDLEGKQGNFKNLREFVFKNIDGYFKIYSKHTPIIGRDFPQGFSALEPLVDLPIGINITRFQTLLALNRSYLTPGDSSQGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGICASYQTQTKSRRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 23 - protein, 1269 aa, SARS-CoV-2 Omicron JN.1
MFVFLVLLPLVSSQCVMPLFNLITTTQSYTNSFTRGVYYPDKVFRSSVLHLTQDLFLPFFSNVTWFHAISGTNGTKRFDNPVLPFNDGVYFASTEKSNIIRGWIFGTTLDSKTQSLLIVNNATNVFIKVCEFQFCNDPFLGVYHKNNKSWMESESGVYSSANNCTFEYVSSPFLMDLEGKQGNFKNLREFVFKNIDGYFKIYSKHTPIIGRDFPQGFSALEPLVDLPIGINITRFQTLLALNRSYLTPGDSSQGWTAGAADYYVGYLQPRTFLLKYNENGTITDAVDCALDPLSETKCTLKSFTVEKGIYQTSNFRVQPTESIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGICASYQTQTKSRRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - protein, 1269 aa, SARS-CoV-2 Omicron JN.1
MFVFLVLLPLVSSQCVMPLFNLITTTQEYTNSFTRGVYYPDKVFRSSVLHMTTDLFLPFFSNVTWFHAISGSNGTKYFDNPVLPFNDGVYFASTEKSNIIRGWIFGTTLDSKTQSLLIVNNATNVFIKVCYFQFCNDPFLGVYHKNNKSWMESESGVYDSANNCTFEYVSSPFLMDLEGKQGNFKNLREFVFKNIDGYFKIYSKHTPIITRDFPQGFSALEPLVDLPIGINITRFQTLLALNRSYLTPGDSSQGWTVGAADYYVGYLQPRTFLLKYNENGTITDAVDCALDPLSETKCTLKSFTVEKGIYQTSNFRVQPTDSIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGICASYQTQTKSRRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 25 - protein, 1269 aa, SARS-CoV-2 Omicron JN.1
MFVFLVLLPLVSSQCVMPLFNLITTTQEYTNSFTRGVYYPDKVFRSSVLHMTTDLFLPFFSNVTWFHAISGSNGTKYFDNPVLPFNDGVYFASTEKSNIIRGWIFGTTLDSKTQSLLIVNNATNVFIKVCYFQFCNDPFLGVYHKNNKSWMESESGVYDSANNCTFEYVSQPFLMDLEGKQGNFKNLREFVFKNIDGYFKIYSKHTPIITRDFPQGFSALEPLVDLPIGINITRFQTLLALNRSYLTPGDSSSGWTVGAADYYVGYLQPRTFLLKYNENGTITDAVDCALDPLSETKCTLKSFTVEKGIYQTSNFRVQPTDSIVRFPNVTNLCPFHEVFNATRFASVYAWNRTRISNCVADYSVLYNFAPFFAFKCYGVSPTKLNDLCFTNVYADSFVIKGNEVSQIAPGQTGNIADYNYKLPDDFTGCVIAWNSNKLDSKHSGNYDYWYRSFRKSKLKPFERDISTEIYQAGNKPCKGKGPNCYFPLQSYGFRPTYGVGHQPYRVVVLSFELLHAPATVCGPKKSTNLVKNKCVNFNFNGLTGTGVLTKSNKKFLPFQQFGRDIVDTTDAVRDPQTLEILDITPCSFGGVSVITPGTNTSNQVAVLYQGVNCTEVSVAIHADQLTPTWRVYSTGSNVFQTRAGCLIGAEYVNNSYECDIPIGAGICASYQTQTKSRRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLFSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQLELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT